SEQUENCE LISTING (65 sequences)

SEQ ID NO: 1 - protein, 47 aa, Homo sapiens
PCSERRKHLFVQDPQTCKCSCKNTDSRCKARQLELNERTCRCDKPRR

SEQ ID NO: 2 - protein, 47 aa, Artificial Sequence
  Description of Artificial Sequence Synthetic polypeptide
  MOD_RES (5)..(8): This position may be a variable amino acid, a non-basic amino acid substitution, deleted, or the residue of the native VEGF polypeptide sequence at that position as shown in SEQ ID NO 1
  MOD_RES (18): This position may be a variable amino acid, a non-basic amino acid substitution, deleted, or the residue of the native VEGF polypeptide sequence at that position as shown in SEQ ID NO 1
  MOD_RES (22): This position may be a variable amino acid, a non-basic amino acid substitution, deleted, or the residue of the native VEGF polypeptide sequence at that position as shown in SEQ ID NO 1
  MOD_RES (27): This position may be a variable amino acid, a non-basic amino acid substitution, deleted, or the residue of the native VEGF polypeptide sequence at that position as shown in SEQ ID NO 1
  MOD_RES (29): This position may be a variable amino acid, a non-basic amino acid substitution, deleted, or the residue of the native VEGF polypeptide sequence at that position as shown in SEQ ID NO 1
  MOD_RES (31): This position may be a variable amino acid, a non-basic amino acid substitution, deleted, or the residue of the native VEGF polypeptide sequence at that position as shown in SEQ ID NO 1
  MOD_RES (38): This position may be a variable amino acid, a non-basic amino acid substitution, deleted, or the residue of the native VEGF polypeptide sequence at that position as shown in SEQ ID NO 1
  MOD_RES (41): This position may be a variable amino acid, a non-basic amino acid substitution, deleted, or the residue of the native VEGF polypeptide sequence at that position as shown in SEQ ID NO 1
  MOD_RES (44): This position may be a variable amino acid, a non-basic amino acid substitution, deleted, or the residue of the native VEGF polypeptide sequence at that position as shown in SEQ ID NO 1
  MOD_RES (46)..(47): This position may be a variable amino acid, a non-basic amino acid substitution, deleted, or the residue of the native VEGF polypeptide sequence at that position as shown in SEQ ID NO 1
PCSEXXXXLFVQDPQTCXCSCXNTDSXCXAXQLELNEXTCXCDXPXX

SEQ ID NO: 3 - protein, 47 aa, Artificial Sequence
  Description of Artificial Sequence Synthetic polypeptide
PCSERAKHLFVQDPQTCKCSCKNTDSRCKARQLELNERTCACDKPRR

SEQ ID NO: 4 - protein, 47 aa, Artificial Sequence
  Description of Artificial Sequence Synthetic polypeptide
PCSEAAKHLFVQDPQTCKCSCKNTDSRCKARQLELNERTCACDKPRR

SEQ ID NO: 5 - protein, 47 aa, Artificial Sequence
  Description of Artificial Sequence Synthetic polypeptide
PCSERRKHLFVQDPQTCKCSCANTDSACKAAQLELNERTCRCDKPRR

SEQ ID NO: 6 - protein, 47 aa, Artificial Sequence
  Description of Artificial Sequence Synthetic polypeptide
PCSERRKHLFVQDPQTCKCSCKNTDSACKAAQLELNERTCRCDKPRR

SEQ ID NO: 7 - protein, 47 aa, Artificial Sequence
  Description of Artificial Sequence Synthetic polypeptide
PCSERRKHLFVQDPQTCKCSCANTDSRCKARQLELNERTCRCDKPRR

SEQ ID NO: 8 - protein, 47 aa, Artificial Sequence
  Description of Artificial Sequence Synthetic polypeptide
PCSERRKHLFVQDPQTCKCSCANTDSACKAAQLELNERTCACDKPRR

SEQ ID NO: 9 - protein, 47 aa, Artificial Sequence; identical to SEQ ID NO: 7
  Description of Artificial Sequence Synthetic polypeptide

SEQ ID NO: 10 - protein, 47 aa, Artificial Sequence
  Description of Artificial Sequence Synthetic polypeptide
PCSERRKHLFVQDPQTCKCSCKNTDSRCKARQLELNEATCACDKPRR

SEQ ID NO: 11 - protein, 47 aa, Artificial Sequence
  Description of Artificial Sequence Synthetic polypeptide
PCSEAAKHLFVQDPQTCKCSCKNTDSRCKARQLELNERTCRCDKPRR

SEQ ID NO: 12 - protein, 47 aa, Artificial Sequence
  Description of Artificial Sequence Synthetic polypeptide
PCSEAAKHLFVQDPQTCKCSCKNTDSRCKARQLELNEATCACDKPRR

SEQ ID NO: 13 - protein, 55 aa, Artificial Sequence
  Description of Artificial Sequence Synthetic polypeptide
ARQENPCGPCSERAKHLFVQDPQTCKCSCKNTDSRCKARQLELNERTCACDKPRR

SEQ ID NO: 14 - protein, 55 aa, Artificial Sequence
  Description of Artificial Sequence Synthetic polypeptide
ARQENPCGPCSEAAKHLFVQDPQTCKCSCKNTDSRCKARQLELNERTCACDKPRR

SEQ ID NO: 15 - protein, 55 aa, Artificial Sequence
  Description of Artificial Sequence Synthetic polypeptide
ARQENPCGPCSERRKHLFVQDPQTCKCSCANTDSACKAAQLELNERTCRCDKPRR

SEQ ID NO: 16 - protein, 55 aa, Artificial Sequence
  Description of Artificial Sequence Synthetic polypeptide
ARQENPCGPCSERRKHLFVQDPQTCKCSCKNTDSACKAAQLELNERTCRCDKPRR

SEQ ID NO: 17 - protein, 55 aa, Artificial Sequence
  Description of Artificial Sequence Synthetic polypeptide
ARQENPCGPCSERRKHLFVQDPQTCKCSCANTDSRCKARQLELNERTCRCDKPRR

SEQ ID NO: 18 - protein, 55 aa, Artificial Sequence
  Description of Artificial Sequence Synthetic polypeptide
ARQENPCGPCSERRKHLFVQDPQTCKCSCANTDSACKAAQLELNERTCACDKPRR

SEQ ID NO: 19 - protein, 55 aa, Artificial Sequence; identical to SEQ ID NO: 17
  Description of Artificial Sequence Synthetic polypeptide

SEQ ID NO: 20 - protein, 55 aa, Artificial Sequence
  Description of Artificial Sequence Synthetic polypeptide
ARQENPCGPCSERRKHLFVQDPQTCKCSCKNTDSRCKARQLELNEATCACDKPRR

SEQ ID NO: 21 - protein, 55 aa, Artificial Sequence
  Description of Artificial Sequence Synthetic polypeptide
ARQENPCGPCSEAAKHLFVQDPQTCKCSCKNTDSRCKARQLELNERTCRCDKPRR

SEQ ID NO: 22 - protein, 55 aa, Artificial Sequence
  Description of Artificial Sequence Synthetic polypeptide
ARQENPCGPCSEAAKHLFVQDPQTCKCSCKNTDSRCKARQLELNEATCACDKPRR

SEQ ID NO: 23 - protein, 165 aa, Artificial Sequence
  Description of Artificial Sequence Synthetic polypeptide
APMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQENPCGPCSERAKHLFVQDPQTCKCSCKNTDSRCKARQLELNERTCACDKPRR

SEQ ID NO: 24 - protein, 165 aa, Artificial Sequence
  Description of Artificial Sequence Synthetic polypeptide
APMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQENPCGPCSEAAKHLFVQDPQTCKCSCKNTDSRCKARQLELNERTCACDKPRR

SEQ ID NO: 25 - protein, 165 aa, Artificial Sequence
  Description of Artificial Sequence Synthetic polypeptide
APMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQENPCGPCSERRKHLFVQDPQTCKCSCKNTDSRCKARQLELNERTCACDKPRR

SEQ ID NO: 26 - protein, 165 aa, Artificial Sequence
  Description of Artificial Sequence Synthetic polypeptide
APMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQENPCGPCSERAKHLFVQDPQTCKCSCKNTDSRCKARQLELNERTCRCDKPRR

SEQ ID NO: 27 - protein, 165 aa, Artificial Sequence
  Description of Artificial Sequence Synthetic polypeptide
APMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQENPCGPCSEARKHLFVQDPQTCKCSCKNTDSRCKARQLELNERTCRCDKPRR

SEQ ID NO: 28 - protein, 47 aa, Artificial Sequence
  Description of Artificial Sequence Synthetic polypeptide
  MOD_RES (5)..(6): Arg or a non-basic amino acid
  MOD_RES (41): Arg or a non-basic amino acid
  see specification as filed for detailed description of substitutions and preferred embodiments
PCSEXXKHLFVQDPQTCKCSCKNTDSRCKARQLELNERTCXCDKPRR

SEQ ID NO: 29 - protein, 163 aa, Artificial Sequence
  Description of Artificial Sequence Synthetic polypeptide
APMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQENPCGPCSERKHLFVQDPQTCKCSCKNTDSRCKARQLELNERTCCDKPRR

SEQ ID NO: 30 - protein, 162 aa, Artificial Sequence
  Description of Artificial Sequence Synthetic polypeptide
APMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQENPCGPCSEKHLFVQDPQTCKCSCKNTDSRCKARQLELNERTCCDKPRR

SEQ ID NO: 31 - protein, 164 aa, Artificial Sequence
  Description of Artificial Sequence Synthetic polypeptide
APMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQENPCGPCSERRKHLFVQDPQTCKCSCKNTDSRCKARQLELNERTCCDKPRR

SEQ ID NO: 32 - protein, 164 aa, Artificial Sequence
  Description of Artificial Sequence Synthetic polypeptide
APMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQENPCGPCSERKHLFVQDPQTCKCSCKNTDSRCKARQLELNERTCRCDKPRR

SEQ ID NO: 33 - protein, 167 aa, Artificial Sequence
  Description of Artificial Sequence Synthetic polypeptide
APMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQENPCGPCSERARKHLFVQDPQTCKCSCKNTDSRCKARQLELNERTCARCDKPRR

SEQ ID NO: 34 - protein, 168 aa, Artificial Sequence
  Description of Artificial Sequence Synthetic polypeptide
APMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQENPCGPCSEARARKHLFVQDPQTCKCSCKNTDSRCKARQLELNERTCARCDKPRR

SEQ ID NO: 35 - protein, 166 aa, Artificial Sequence
  Description of Artificial Sequence Synthetic polypeptide
APMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQENPCGPCSERRKHLFVQDPQTCKCSCKNTDSRCKARQLELNERTCARCDKPRR

SEQ ID NO: 36 - protein, 166 aa, Artificial Sequence
  Description of Artificial Sequence Synthetic polypeptide
APMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQENPCGPCSERARKHLFVQDPQTCKCSCKNTDSRCKARQLELNERTCRCDKPRR

SEQ ID NO: 37 - protein, 166 aa, Artificial Sequence
  Description of Artificial Sequence Synthetic polypeptide
APMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQENPCGPCSEARRKHLFVQDPQTCKCSCKNTDSRCKARQLELNERTCRCDKPRR

SEQ ID NO: 38 - protein, 55 aa, Homo sapiens
ARQENPCGPCSERRKHLFVQDPQTCKCSCKNTDSRCKARQLELNERTCRCDKPRR

SEQ ID NO: 39 - DNA, 40 bp, Artificial Sequence
  Description of Artificial Sequence Synthetic primer
tgtgagccttgttcagaggcggcaaagcatttgtttgtcc

SEQ ID NO: 40 - DNA, 37 bp, Artificial Sequence
  Description of Artificial Sequence Synthetic primer
gagccttgttcagagcgggcaaagcatttgtttgtcc

SEQ ID NO: 41 - DNA, 33 bp, Artificial Sequence
  Description of Artificial Sequence Synthetic primer
aacgaacgtacttgcgcatgtgacaagccgagg

SEQ ID NO: 42 - DNA, 39 bp, Artificial Sequence
  Description of Artificial Sequence Synthetic primer
catttgtttgtcgcagatccggcgacgtgtaaatgttcc

SEQ ID NO: 43 - DNA, 35 bp, Artificial Sequence
  Description of Artificial Sequence Synthetic primer
gtccaagatccgcagacgtgtgcatgttcctgcaa

SEQ ID NO: 44 - DNA, 32 bp, Artificial Sequence
  Description of Artificial Sequence Synthetic primer
cgtgtaaatgttcctgcgcaaacacagactcg

SEQ ID NO: 45 - DNA, 43 bp, Artificial Sequence
  Description of Artificial Sequence Synthetic primer
aacacagactcggcttgcaaggcggcgcagcttgagttaaacg

SEQ ID NO: 46 - DNA, 40 bp, Artificial Sequence
  Description of Artificial Sequence Synthetic primer
tgagttaaacgaagctacttgcgcatgtgacaagccgagg

SEQ ID NO: 47 - DNA, 1723 bp, Homo sapiens
tcgcggaggcttggggcagccgggtagctcggaggtcgtggcgctgggggctagcaccagcgctctgtcgggaggcgcagcggttaggtggaccggtcagcggactcaccggccagggcgctcggtgctggaatttgatattcattgatccgggttttatccctcttcttttttcttaaacatttttttttaaaactgtattgtttctcgttttaatttatttttgcttgccattccccacttgaatcgggccgacggcttggggagattgctctacttccccaaatcactgtggattttggaaaccagcagaaagaggaaagaggtagcaagagctccagagagaagtcgaggaagagagagacggggtcagagagagcgcgcgggcgtgcgagcagcgaaagcgacaggggcaaagtgagtgacctgcttttgggggtgaccgccggagcgcggcgtgagccctcccccttgggatcccgcagctgaccagtcgcgctgacggacagacagacagacaccgcccccagccccagctaccacctcctccccggccggcggcggacagtggacgcggcggcgagccgcgggcaggggccggagcccgcgcccggaggcggggtggagggggtcggggctcgcggcgtcgcactgaaacttttcgtccaacttctgggctgttctcgcttcggaggagccgtggtccgcgcgggggaagccgagccgagcggagccgcgagaagtgctagctcgggccgggaggagccgcagccggaggagggggaggaggaagaagagaaggaagaggagagggggccgcagtggcgactcggcgctcggaagccgggctcatggacgggtgaggcggcggtgtgcgcagacagtgctccagccgcgcgcgctccccaggccctggcccgggcctcgggccggggaggaagagtagctcgccgaggcgccgaggagagcgggccgccccacagcccgagccggagagggagcgcgagccgcgccggccccggtcgggcctccgaaaccatgaactttctgctgtcttgggtgcattggagccttgccttgctgctctacctccaccatgccaagtggtcccaggctgcacccatggcagaaggaggagggcagaatcatcacgaagtggtgaagttcatggatgtctatcagcgcagctactgccatccaatcgagaccctggtggacatcttccaggagtaccctgatgagatcgagtacatcttcaagccatcctgtgtgcccctgatgcgatgcgggggctgctgcaatgacgagggcctggagtgtgtgcccactgaggagtccaacatcaccatgcagattatgcggatcaaacctcaccaaggccagcacataggagagatgagcttcctacagcacaacaaatgtgaatgcagaccaaagaaagatagagcaagacaagaaaaaaaatcagttcgaggaaagggaaaggggcaaaaacgaaagcgcaagaaatcccggtataagtcctggagcgttccctgtgggccttgctcagagcggagaaagcatttgtttgtacaagatccgcagacgtgtaaatgttcctgcaaaaacacagactcgcgttgcaaggcgaggcagcttgagttaaacgaacgtacttgcagatgtgacaagccgaggcggtgagccgggcaggaggaaggagcctccctcagggtttcgg

SEQ ID NO: 48 - protein, 215 aa, Homo sapiens
MNFLLSWVHWSLALLLYLHHAKWSQAAPMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQEKKSVRGKGKGQKRKRKKSRYKSWSVPCGPCSERRKHLFVQDPQTCKCSCKNTDSRCKARQLELNERTCRCDKPRR

SEQ ID NO: 49 - protein, 47 aa, Artificial Sequence; identical to SEQ ID NO: 2
  Description of Artificial Sequence Synthetic polypeptide
  MOD_RES (5)..(8): This position may be a variable amino acid or a non-basic amino acid substitution
  MOD_RES (18): This position may be a variable amino acid or a non-basic amino acid substitution
  MOD_RES (22): This position may be a variable amino acid or a non-basic amino acid substitution
  MOD_RES (27): This position may be a variable amino acid or a non-basic amino acid substitution
  MOD_RES (29): This position may be a variable amino acid or a non-basic amino acid substitution
  MOD_RES (31): This position may be a variable amino acid or a non-basic amino acid substitution
  MOD_RES (38): This position may be a variable amino acid or a non-basic amino acid substitution
  MOD_RES (41): This position may be a variable amino acid or a non-basic amino acid substitution
  MOD_RES (44): This position may be a variable amino acid or a non-basic amino acid substitution
  MOD_RES (46)..(47): This position may be a variable amino acid or a non-basic amino acid substitution

SEQ ID NO: 50 - protein, 47 aa, Artificial Sequence; identical to SEQ ID NO: 2
  Description of Artificial Sequence Synthetic polypeptide
  MOD_RES (5)..(6): This position may be a variable amino acid or a non-basic amino acid substitution
  MOD_RES (7)..(8): Variable amino acid
  MOD_RES (18): This position may be a variable amino acid or a non-basic amino acid substitution
  MOD_RES (22): This position may be a variable amino acid or a non-basic amino acid substitution
  MOD_RES (27): This position may be a variable amino acid or a non-basic amino acid substitution
  MOD_RES (29): This position may be a variable amino acid or a non-basic amino acid substitution
  MOD_RES (31): This position may be a variable amino acid or a non-basic amino acid substitution
  MOD_RES (38): This position may be a variable amino acid or a non-basic amino acid substitution
  MOD_RES (41): This position may be a variable amino acid or a non-basic amino acid substitution
  MOD_RES (44): Variable amino acid
  MOD_RES (46)..(47): Variable amino acid

SEQ ID NO: 51 - protein, 47 aa, Artificial Sequence; identical to SEQ ID NO: 2
  Description of Artificial Sequence Synthetic polypeptide
  MOD_RES (5)..(8): This position may be a variable amino acid or deleted
  MOD_RES (18): This position may be a variable amino acid or deleted
  MOD_RES (22): This position may be a variable amino acid or deleted
  MOD_RES (27): This position may be a variable amino acid or deleted
  MOD_RES (29): This position may be a variable amino acid or deleted
  MOD_RES (31): This position may be a variable amino acid or deleted
  MOD_RES (38): This position may be a variable amino acid or deleted
  MOD_RES (41): This position may be a variable amino acid or deleted
  MOD_RES (44): This position may be a variable amino acid or deleted
  MOD_RES (46)..(47): This position may be a variable amino acid or deleted

SEQ ID NO: 52 - protein, 47 aa, Artificial Sequence; identical to SEQ ID NO: 2
  Description of Artificial Sequence Synthetic polypeptide
  MOD_RES (5)..(6): This position may be a variable amino acid or deleted
  MOD_RES (7)..(8): Variable amino acid
  MOD_RES (18): This position may be a variable amino acid or deleted
  MOD_RES (22): This position may be a variable amino acid or deleted
  MOD_RES (27): This position may be a variable amino acid or deleted
  MOD_RES (29): This position may be a variable amino acid or deleted
  MOD_RES (31): This position may be a variable amino acid or deleted
  MOD_RES (38): This position may be a variable amino acid or deleted
  MOD_RES (41): This position may be a variable amino acid or deleted
  MOD_RES (44): Variable amino acid
  MOD_RES (46)..(47): Variable amino acid

SEQ ID NO: 53 - protein, 47 aa, Artificial Sequence; identical to SEQ ID NO: 2
  Description of Artificial Sequence Synthetic polypeptide
  MOD_RES (5)..(8): This position may be a variable amino acid or the residue of the native VEGF polypeptide sequence at that position as shown in SEQ ID NO 1
  MOD_RES (18): This position may be a variable amino acid or the residue of the native VEGF polypeptide sequence at that position as shown in SEQ ID NO 1
  MOD_RES (22): This position may be a variable amino acid or the residue of the native VEGF polypeptide sequence at that position as shown in SEQ ID NO 1
  MOD_RES (27): This position may be a variable amino acid or the residue of the native VEGF polypeptide sequence at that position as shown in SEQ ID NO 1
  MOD_RES (29): This position may be a variable amino acid or the residue of the native VEGF polypeptide sequence at that position as shown in SEQ ID NO 1
  MOD_RES (31): This position may be a variable amino acid or the residue of the native VEGF polypeptide sequence at that position as shown in SEQ ID NO 1
  MOD_RES (38): This position may be a variable amino acid or the residue of the native VEGF polypeptide sequence at that position as shown in SEQ ID NO 1
  MOD_RES (41): This position may be a variable amino acid or the residue of the native VEGF polypeptide sequence at that position as shown in SEQ ID NO 1
  MOD_RES (44): This position may be a variable amino acid or the residue of the native VEGF polypeptide sequence at that position as shown in SEQ ID NO 1
  MOD_RES (46)..(47): This position may be a variable amino acid or the residue of the native VEGF polypeptide sequence at that position as shown in SEQ ID NO 1

SEQ ID NO: 54 - protein, 47 aa, Artificial Sequence; identical to SEQ ID NO: 2
  Description of Artificial Sequence Synthetic polypeptide
  MOD_RES (5)..(6): This position may be a variable amino acid or the residue of the native VEGF polypeptide sequence at that position as shown in SEQ ID NO 1
  MOD_RES (7)..(8): Variable amino acid
  MOD_RES (18): This position may be a variable amino acid or the residue of the native VEGF polypeptide sequence at that position as shown in SEQ ID NO 1
  MOD_RES (22): This position may be a variable amino acid or the residue of the native VEGF polypeptide sequence at that position as shown in SEQ ID NO 1
  MOD_RES (27): This position may be a variable amino acid or the residue of the native VEGF polypeptide sequence at that position as shown in SEQ ID NO 1
  MOD_RES (29): This position may be a variable amino acid or the residue of the native VEGF polypeptide sequence at that position as shown in SEQ ID NO 1
  MOD_RES (31): This position may be a variable amino acid or the residue of the native VEGF polypeptide sequence at that position as shown in SEQ ID NO 1
  MOD_RES (38): This position may be a variable amino acid or the residue of the native VEGF polypeptide sequence at that position as shown in SEQ ID NO 1
  MOD_RES (41): This position may be a variable amino acid or the residue of the native VEGF polypeptide sequence at that position as shown in SEQ ID NO 1
  MOD_RES (44): Variable amino acid
  MOD_RES (46)..(47): Variable amino acid

SEQ ID NO: 55 - protein, 47 aa, Artificial Sequence
  Description of Artificial Sequence Synthetic polypeptide
  MOD_RES (41): Non-basic amino acid
  see specification as filed for detailed description of substitutions and preferred embodiments
PCSERRKHLFVQDPQTCKCSCKNTDSRCKARQLELNERTCXCDKPRR

SEQ ID NO: 56 - protein, 47 aa, Artificial Sequence
  Description of Artificial Sequence Synthetic polypeptide
  MOD_RES (6): Non-basic amino acid
  see specification as filed for detailed description of substitutions and preferred embodiments
PCSERXKHLFVQDPQTCKCSCKNTDSRCKARQLELNERTCRCDKPRR

SEQ ID NO: 57 - RNA, 10 nt, Artificial Sequence
  Description of Artificial Sequence Synthetic oligonucleotide
gaagaauugg

SEQ ID NO: 58 - RNA, 8 nt, Artificial Sequence
  Description of Artificial Sequence Synthetic oligonucleotide
uuggacgc

SEQ ID NO: 59 - RNA, 8 nt, Artificial Sequence
  Description of Artificial Sequence Synthetic oligonucleotide
gugaaugc

SEQ ID NO: 60 - RNA, 27 nt, Artificial Sequence
  Description of Artificial Sequence Synthetic oligonucleotide
cggaaucagugaaugcuuauacauccg

SEQ ID NO: 61 - RNA, 27 nt, Artificial Sequence
  Description of Artificial Sequence Synthetic oligonucleotide
  modified_base (1): 2′-fluorocytidylic acid
  modified_base (2)..(3): 2′-methoxyguanylic acid
  modified_base (4)..(5): Riboadenylic acid
  modified_base (6): 2′-flourouridylic acid
  modified_base (7): 2′-fluorocytidylic acid
  modified_base (8): 2′-methoxyadenylic acid
  modified_base (9): 2′-methoxyguanylic acid
  modified_base (10): 2′-flourouridylic acid
  modified_base (11): 2′-methoxyguanylic acid
  modified_base (12)..(13): 2′-methoxyadenylic acid
  modified_base (14): 2′-flourouridylic acid
  modified_base (15): 2′-methoxyguanylic acid
  modified_base (16): 2′-fluorocytidylic acid
  modified_base (17)..(18): 2′-flourouridylic acid
  modified_base (19): 2′-methoxyadenylic acid
  modified_base (20): 2′-flourouridylic acid
  modified_base (21): 2′-methoxyadenylic acid
  modified_base (22): 2′-fluorocytidylic acid
  modified_base (23): 2′-methoxyadenylic acid
  modified_base (24): 2′-flourouridylic acid
  modified_base (25)..(26): 2′-fluorocytidylic acid
  modified_base (27): 2′-methoxyguanylic acid
cggaaucagugaaugcuuauacauccg

SEQ ID NO: 62 - protein, 47 aa, Artificial Sequence
  Description of Artificial Sequence Synthetic polypeptide
PCSEAAAALFVQDPQTCACSCANTDSACAAAQLELNEATCACDAPAA

SEQ ID NO: 63 - protein, 47 aa, Artificial Sequence
  Description of Artificial Sequence Synthetic polypeptide
PCSERRKHLFVADPATCKCSCKNTDSRCKARQLELNERTCRCDKPRR

SEQ ID NO: 64 - protein, 47 aa, Artificial Sequence
  Description of Artificial Sequence Synthetic polypeptide
PCSERRKHLFVQDPQTCACSCKNTDSRCAARQLELNERTCRCDKPRR

SEQ ID NO: 65 - protein, 47 aa, Artificial Sequence
  Description of Artificial Sequence Synthetic polypeptide
PCSERRKHLFVQDPQTCACSCKNTDSRCKARQLELNERTCRCDKPRR

(19) United States
(12) Patent Application Publication
(10) Pub. No.: US 2006/0286636 A1
(43) Pub. Date: Dec. 21, 2006

(54) VEGF variants

(76) Inventors: David T. Shima, Barnet (GB); Anthony P. Adamis, Bronxville, NY (US); Gregory S. Robinson, Wilmington, MA (US); Yin-Shan Ng, North Billerica, MA (US); Kazuaki Nishijima, Arlington, MA (US); Dominik Krilleke, Arlington, MA (US)

Correspondence Address: (OSI) EYETECH, INC., 3 TIMES SQUARE 12TH FLOOR, NEW YORK, NY, 10036, US

(21) Appl. No.: 11/414,724

(22) Filed: Apr. 28, 2006

Related U.S. Application Data
(60) Provisional application No. 60/676,355, filed on Apr. 29, 2005.

(51) Int. Cl. C07K 14/475; C07H 21/04; C12P 21/06; A61K 38/18

(52) U.S. Cl. 435/69.1; 514/12; 435/320.1; 435/325; 530/399; 536/23.5

(57) ABSTRACT

Applicants have defined the pro-inflammatory domain of the Vascular Endothelial Growth Factor VEGF164(165) protein molecule using VEGF164 protein mutants in which the heparin binding domain is inactivated through alanine scanning, site directed mutagenesis. The invention provides novel VEGF variants having a modified heparin binding domain. The VEGF variants modified heparin binding function compared to native VEGF while maintaining receptor binding function. The invention provides compositions and methods for treating disorders relating to angiogenesis and inflammation.

### RELATED APPLICATION

This Application claims the benefit of U.S. Provisional Application No. 60/676,355, filed on Apr. 29, 2005. The entire teachings of the above application is incorporated herein by reference.

### FIELD OF THE INVENTION

The invention relates to medicine. More specifically, the invention relates to angiogenesis and neovascularization, and more particularly the invention relates to variants of vascular endothelial growth factor (VEGF). The compositions and methods disclosed herein are useful for treating disorders relating to angiogenesis and inflammation.

### BACKGROUND OF THE INVENTION

Angiogenesis, or neovascularization, is the process by which new blood vessels develop from existing endothelium. Normal angiogenesis plays an important role in a variety of processes including embryonic development, wound healing and several components of female reproductive function, however angiogenesis is also associated with certain pathological conditions. Undesirable or pathological angiogenesis has been associated with certain disease states including proliferative retinopathies, rheumatoid arthritis, psoriasis and cancer (see Fan et al. (1995) Trends Pharmacol. Sci. 16: 57; and Folkman (1995) Nature Medicine 1: 27). Indeed the quantity of blood vessels in tumor tissue is a strong negative prognostic indicator in breast cancer (Weidner et al. (1992) J. Natl. Cancer Inst. 84:1875-1887), prostate cancer (Weidner et aL. (1993) Am. J. Pathol. 143:401-409), brain tumors (Li et al. (1994) Lancet 344:82-86), and melanoma (Foss et al. (1996) Cancer Res. 56:2900-2903). Furthermore, the alteration of vascular permeability is thought to play a role in both normal and pathological physiological processes (Cullinan-Bove et al. (1993) Endocrinol. 133: 829; Senger et al. (1993) Cancer and Metastasis Reviews 12: 303).

Vascular Endothelial Growth Factor (VEGF) has been established as the prime angiogenic molecule during development, adult physiology and pathology. VEGF binds VEGFR-1 and VEGFR-2 as well as neuropilin-1 (Nrp-1) and Nrp-2; the latter are receptors for semaphorins, molecules involved in axonal guidance during neuronal development (Kolodkin et al. (1997) Cell, 90:753-62; Chen et al. (1997) Neuron, 19:547-59). VEGF induces proliferation, sprouting, migration and tube formation of endothelial cells (ECs) (Ferrara et al. (2003) Nat. Med., 9:669-76). VEGF is also a potent survival factor for ECs during physiological and tumor angiogenesis and it has been shown to induce the expression of antiapoptotic proteins in the ECs (Benjamin et al. (1997) Proc. Natl. Acad. Sci. U.S.A., 94:8761-6; Gerber et al. (1998) J. Biol. Chem., 273:13313-6). VEGF was originally described as a permeability factor, as it increases permeability of the endothelium through the formation of intercellular gaps, vesico-vascular organelles, vacuoles and fenestrations (Bates et al. (2002) J. Anat., 200:581-97). VEGF also causes vasodilatation through the induction of the endothelial nitric oxide synthase (eNOS) and the subsequent increase in nitric oxide production (Hood et al. (1998) Am. J. Physiol., 274:H1054-8; Kroll et al. (1998) Biochem. Biophys. Res. Commun., 265:636-99)

Although VEGF acts mostly on ECs, it has been shown to also bind VEGF receptors on hematopoietic stem cells (HSCs), monocytes, osteoblasts and neurons (Ferrara et al. (2003) Nat. Med., 9:669-76). Besides angiogenesis, VEGF induces HSC mobilization from the bone marrow, monocyte chemoattraction, osteoblast-mediated bone formation and neuronal protection (Ferrara et al. (2003) Nat. Med., 9:669-76) (Storkebaum et al. (2004) BioEssays, 26:943-54). Furthermore, VEGF stimulates inflammatory cell recruitment and promotes the expression of proteases implicated in pericellular matrix degradation in angiogenesis (Pepper et al. (1991) Biochem Biophys. Res. Commun., 181:902-6; Unemori et al., (1992) J. Cell. Physiol., 153:557-62; Mandriota et al. (1995) J. Biol. Chem., 270:9709-16). Many cytokines including platelet-derived growth factor, epidermal growth factor, basic fibroblast growth factor and transforming growth factors induce VEGF expression in cells (Ferrara, N. (2004) Endocr. Rev., 25:581-611).

VEGF stimulates axonal outgrowth, improves the survival of superior cervical and dorsal route ganglion neurons, and enhances the survival of mesencephalic neurons in organotypic explant cultures (Sondell, M et al., J. Neurosci., (1999) 19:5731-5740; Sondell, M et al.,(2000) Eur. J. Neurosci. 12:4243-4254), illustrating the protective effect of VEGF. Furthermore, VEGF can rescue HN33 hippocampal cells from apopotosis induced by serum withdrawal (Jin, K L, et al., (2000), Proc Natl Acad Sci. 97(18):10242-7.). Low VEGF levels may cause motor neuron degeneration and local delivery of VEGF could protect motoneurons and prolong their survival (Oosthuyse B et al.,(2001) Nat Genet. 28(2):131-8; Storkebaum E, et al., (2005) Nat Neurosci. 8(1):85-92). VEGF also has direct protective effect to a certain kind of neuronal cells against NMDA-induced toxicity (Matsuzaki H, et al., (2001), FASEB J. 15(7):1218-20.) or ischemic insult.

At least six VEGF isoforms of variable amino acid number are produced through alternative splicing: VEGF121, VEGF145, VEGF 165, VEGF183, VEGF189 and VEGF206 (Table 1) (Ferrara et al. (2003) Nat. Med., 9:669-76). VEGF121, VEGF165 and VEGF 189 are the major forms secreted by most cell types (Robinson et al. (2001) J. Cell. Sci., 114:853-65). After secretion, VEGF121 may diffuse relatively freely in tissues, while approximately half of the secreted VEGF165 binds to cell surface heparin sulfate proteroglycans (HSPGs). VEGF189 remains almost completely sequestered by HSPGs in the extracellular matrix making HSPGs a reservoir of VEGF that can be mobilized via proteolysis (Ferrara et al. (2003) Nat. Med., 9:669-76).

VEGF is first expressed mainly in the anterior portion of mouse embryos where it directs the migration of VEGFR-1 and VEGFR-2 positive cells in embryonic tissues (Hiratsuka et al. (2005) Mol. Cell. Biol., 25:355-63). In general, VEGF expression is stronger at sites of active vasculogenesis and angiogenesis in embryos (Weinstein, B M (1999) Dev. Dyn., 215:2-11). Homozygous VEGF knockout mice die at E8-E9 from defects in blood island formation, EC development and vascular formation (Ferrara, N. (2004) Endocr. Rev., 25:581-611). The levels of VEGF protein during development appear critical as mice lacking even a single VEGF allele die at E11-E12, displaying defects in early vascular development (Ferrara, N. (2004) Endocr. Rev., 25:581-611). The different biological functions of VEGF isoforms were illustrated by studies on isoform-specific VEGF knockout mice. Mice expressing only VEGF120 (homolog of human VEGF 121) die soon after birth and those that survive succumb to ischemic cardiomyopathy and multiorgan failure (Carmeliet et al. (1999) Nat. Med., 5:495-502). Mice expressing only VEGF188 (human VEGF189) display impaired arteriolar development and approximately half die at birth (Stalmans et al. (2002) J. Clin. Invest., 109:327-36). Mice expressing only VEGF164 (human VEGF165) are viable and healthy (Stalmans et al. (2002) J. Clin. Invest., 109:327-36). These studies underline the importance of VEGF 165 as the principal effector of VEGF action, with intermediate diffusion and matrix-binding properties.

VEGF is strongly induced in hypoxic conditions via hypoxia inducible factor (HIF) regulated elements of the VEGF gene (Pugh et al. (2003) Nat. Med., 9:677-84). Constitutive degradation of hypoxia inducible factor (HIF)-1α is blocked in hypoxia because of the oxygen requirement of HIF prolyl hydroxylases, followed by stabilization of HIF-1α a and its heterodimerization of the HIF-1β, also called the aryl hydrocarbon nuclear translocator (ARNT). These complexes then bind hypoxia-responsive elements (HREs) in the promoters of hypoxia inducible genes and initiate transcription of a set of more than a hundred genes, including genes involved in glucose transport, glycolysis, and angiogenesis (Pugh et al. (2003) Nat. Med., 9:677-84; Luttun et al. (2002) Biochem Biophys. Res. Commun., 295:428-34). Interestingly, Bartonella henselae, the causative agent of cat-scratch fever, can induce hypoxia via an intracellular oxygen consumption mechanism, leading to VEGF induction and an angiomatous tumor (Kempf et al. (2005) Circ. Res., 3:623-32). Examples of other hypoxia-regulated genes include cyclooxygenase-2 (COX-2), MMP-2, VEGF and VEGFR- 1 (Pugh et al. (2003) Nat. Med., 9:677-84). Deletion of a HRE from the mouse VEGF gene promoter results in progressive motoneuron degeneration, presumably due to insufficient vascular perfusion of nervous tissue and impaired motoneuron survival via loss of VEGF induction (Oosthuyse et al. (2001) Nat. Genet., 28:131-8).

The skin has been widely used as a model for studying VEGF action in vivo; for example, transgenic mice overexpressing VEGF in the skin have abundant cutaneous angiogenesis and an inflammatory skin condition resembling psoriasis (Xia et al. (2003) Blood, 102:161-8). Overexpression of VEGF in mouse skin also accelerates experimental tumor growth (Larcher et al. (1998) Oncogene, 17:303-11). In contrast, mice with a targeted deletion of VEGF in the epidermis exhibit delayed wound healing, while chemically induced skin papillomas developed less frequently in these animals (Rossiter et al. (2004) Cancer Res., 64:3508-16). VEGF blocking monoclonal antibodies or VEGF receptor inhibition reduce the growth of experimental tumors in mice and humans (Ferrara, N (2004) Endocr. Rev., 25:581-611; Sepp-Lorenzino et al. (2004) Cancer Res., 64:751-6; Kabbinavar et al. (2003) J. Clin. Oncol., 21:60-5). In humans, VEGF is expressed in practically all solid tumors studied as well as in some hematological malignancies (Ferrara et al. (2003) Nat. Med., 9:669-76). In fact, correlations have been found between the level of VEGF expression, disease progression and survival (Ferrara, N. (2002) Semin. Oncol., 29:10-4).

The effects of VEGF on the lymphatic vasculature have also been recently studied. Adenoviral overexpression of the murine VEGF164 in the skin induced formation of giant lymphatic vessels (Nagy et al. (2002) J. Exp. Med., 196:1497-506), while another study employing the human VEGF165 isoform reported only dilatation of cutaneous lymphatics (FIG. 3A) (Saaristo et al. (2002) FASEB J., 16:1041-9). However, VEGF did not induce lymphangiogenesis in a number of other tissue types (Kubo et al. (2002) Proc. Natl. Acad. Sci. U.S.A., 99:8868-73; Rissanen et al. (2003) Circ. Res., 92:1098-106; Cao et al. (2004) Circ. Res., 94:664-70; Lee et al. (2004) Nat. Med., 10:1095-103). The lymphangiogenic effects of VEGF may be linked to the recruitment of inflammatory cells, such as macrophages, which express VEGFR-1 and secrete lymphangiogenic factors (Clauss et al. (1996) J. Biol. Chem., 271:17629-34; Rafii et al. (2003) Ann. N.Y Acad. Sci., 996:49-60; Cursiefen et al. (2004) J. Clin. Invest., 113:1040-50). At least in midgestation mouse embryos, VEGF-C but not VEGF had the capacity to induce migration of endothelial cells committed to the lymphatic endothelial lineage (Karkkainen et al. (2004) Nat. Immunol., 5:74-80).

VEGF also plays an important role in ocular health and disease and is responsible in large part for the physiological and pathological development of retinal vasculature (A. P. Adamis et al. (2005) Retina, 25:111-118; Y.-S. Ng et al. (2006) Experimental Cell Research, 312: 527-537; E. W. M. Ng et al. (2006) Nature Reviews, 5:123-132). VEGF has at least five isoforms generated through the alternative splicing of mRNA arising from a single gene. The two major prevalent isoforms in the retina are VEGF121(120) and VEGF165(164). The human proteins are one residue longer than the murine homologues.

Leukocytes have been shown to be beneficial for ocular health because they prune the retinal vasculature during normal development. However S. Ishida et al. have shown that leukocytes obliterate the retinal vasculature in disease (Nature Medicine (2003) 9:781-788). Extensive leukocyte adhesion has been observed at the leading edge of pathological, but not physiological, neovascularization. Studies have demonstrated that ischemia-induced retinal neovascularization is caused in part by a local inflammatory response. During pathological neovascularization, both the absolute and relative expression levels for VEGF164(165) increased to a greater degree than during physiological neovascularization. VEGF164(165) has been identified as a pro-inflammatory isoform that was found to be significantly more potent at inducing leukocyte recruitment and inflammation than other VEGF isoforms. VEGF164(165) was also found to be more potent at inducing the chemotaxis of monocytes, an effect mediated by VEGFR-1. In an immortalized human leukocyte cell line, VEGF164(165) was found to induce tyrosine phosphorylation of VEGFR-1 more efficiently. (See Investigative Ophthalmology & Visual Science (February 2004) 45:368-374.)

Leukocytes, a non-endothelial cell type, have also been shown to contribute to VEGF-induced vascular permeability. Using a rat model, it was shown that intravitreous injections so that a retina is bathed in pathophysiological concentrations of VEGF precipitate an extensive retinal leukocyte stasis (leukostasis) that coincides with vascular changes such as the increased vascular permeability and capillary non-perfusion. In experimental diabetes, the increased presence of static leukocytes in the retinal circulation is correlated with increased vascular permeability. Leukostasis and vascular permeability changes coincide with the upregulation of retinal Intercellular Adhesion Molecule-1 (ICAM-1). When ICAM-1 bioactivity is blocked with an antibody, increases in retinal leukostasis and vascular permeability are reduced by 49% and 86%, respectively. (See American Journal of Pathology (2000) 156:1733-1739.)

Macular edema is one of the greatest sources of vision loss in diabetes and it can appear at any time during the course of diabetic retinopathy. Diabetic retinopathy is a pathologic condition that is a direct consequence of blood-retinal barrier (BRB) breakdown. Retinal leukostasis and leakage correlated closely and increased with the duration of diabetes. In eyes with early diabetes, the expression of retinal VEGF164 is eleven times greater than VEGF120. VEGF-induced BRB breakdown is mediated, in part, through ICAM-1-dependent retinal leukostasis. In vitro and in vivo data also show that VEGF165 more potently induces endothelial ICAM-1 expression, as well as leukocyte adhesion and migration. On an equimolar basis, VEGF164 is at least twice as potent as VEGF120 at increasing ICAM-1 levels and inducing ICAM-1-mediated retinal leukostasis and BRB breakdown in vivo. The isoform-specific blockade of endogenous VEGF164 with Macugen (pegaptanib sodium) resulted in a significant suppression of retinal leukostasis and BRB breakdown in both early and established diabetes. Macugen® potently suppressed leukocyte adhesion and pathological neovascularization, whereas it had little or no effect on physiological neovascularization. (See Investigative Ophthalmology & Visual Science (2003) 44:2155-2162). Likewise, genetically altered VEGF164-deficient (VEGF120/188) mice exhibited no difference in physiological neovascularization when compared with wild-type (VEGF+/+) controls. (See The Journal of Experimental Medicine (2003) 198:483-489.)

Structure elucidation of VEGF has been reported. The first crystal structure of VEGF was reported by Y. A. Muller et al. (Structure (1997) 5:1325-1338). Shortly thereafter, C. Wiesmann et al. reported a crystal structure at 1.7 Å resolution of VEGF in complex with Domain 2 of the Flt-1 Receptor (Cell (1997) 91:695-704) and M. A. McTigue et al. reported a crystal structure of the kinase domain of VEGF (Structure (1999) 7:319-330). Melissa E. Stauffer et al. elucidatated a solution structure of the VEGF heparin binding domain (Journal of Biomolecular NMR (2002) 23:57-61).

Studies comparing the molecular interactions of full-length VEGF 164 and the Heparin binding domain of VEGF164 have been reported. NMR spectroscopy compared an isolated HBD-Aptamer complex with a full length VEG164-aptamer complex (Lee et al. PNAS, (2005) Vol. 102, 18902-18907, the contents of which is incorporated herein by reference in its entirety).

Variants of VEGF have been reported. T. Zioncheck et al. describe variants of VEGF that include a truncated heparin binding domain (U.S. Pat. No. 6,485,942 and U.S. Patent Application Publication No. 2003/0032145) and N. S. Pollitt et al. describe variants of VEGF that include substituting cysteine amino acid residues for other amino acid residues (U.S. Pat. No. 6,475,796).

Much has been learned about angiogenesis and leukocyte recruitment accompanying development, wound healing and tumor formation. However, the association between VEGF, angiogenesis and leukocyte recruitment remained elusive. The pro-inflammatory domain of the Vascular Endothelial Growth Factor was not known. Therefore, although advances in the understanding of the molecular events accompanying neovascularization have been made, there exists a need to use this understanding to develop further methods and formulations for treating neovascular disorders, including leukostasis and ocular neovascular diseases such as those that occur with Age Related Macular Degeneration (AMD) and Diabetic Retinopathy (DR).

### SUMMARY OF THE INVENTION

The invention is based, in part, upon the finding that the Heparin Binding Domain (HBD) of VEGF is associated with leukocyte recruitment and vascular permeability. In other aspects, the invention is based, in part, upon the finding that Neuropilin (Np-1) is associated with the VEGF mediated pro-inflammatory effects. In other aspects, the invention is based, in part, upon the finding that VEGFR1 (Flt-1) is associated with the VEGF mediated pro-inflammatory effects. Applicants have defined a pro-inflammatory domain of the Vascular Endothelial Growth Factor VEGF164/165 protein molecule using VEGF164 protein mutants in which the heparin binding domain is inactivated through alanine scanning, site directed mutagenesis.

In one aspect, the invention provides novel VEGF variants. The VEGF variants comprise a polypeptide having a modified heparin binding domain. In one embodiment, the heparin binding domain is modified by substituting basic amino acid residues with neutral amino acid residues or acidic amino acid residues. In another embodiment, the heparin binding domain is modified by inserting a non-basic amino acids adjacent to a basic amino acids. In another embodiment, the heparin binding domain is modified by deleteing basic amino acids.

In a particularly useful aspect, the invention provides a polypeptide comprising a VEGF polypeptide sequence variant with reduced pro-inflammatory activity having one or more alterations of a native VEGF polypeptide sequence that reduces heparin binding affinity, while substantially maintaining the affinity for VEGR-2 (FLK-1/KDR). In certain embodiments, the native VEGF polypeptide sequence is human VEGF165. In other embodiments, the native VEGF polypeptide sequence is human VEGF189. In further embodiments, the native VEGF polypeptide sequence is human VEGF206. In still other embodiments, the native VEGF polypeptide sequence is mouse VEGF164. In still further embodiments, the native VEGF polypeptide sequence is a VEGF isoform of a mammal such as a human, a mouse, a rat, a monkey, a cow, a pig, a sheep, a dog, a cat, or a rabbit.

In yet another aspect, the invention provides a polypeptide that includes a VEGF polypeptide sequence variant having one or more amino acid substitutions, amino acid insertions and/or amino acid deletions of the native VEGF polypeptide sequence PCSERRKHLF VQDPQTCKCS CKNTDSRCKA RQLELNERTC RCDKPRR (Seq. ID No.1). In certain embodiments, the polypeptide includes one or more substitutions of a basic amino acid of the native VEGF polypeptide sequence with a non-basic amino acid. In other embodiments, the polypeptide includes one or more deletions of a basic amino acid of the native VEGF polypeptide sequence. In other embodiments, the polypeptide includes one or more insertions of a non-basic amino acid adjacent to a basic amino acid of the native VEGF polypeptide sequence. In other embodiments, the polypeptide includes a combination of substitutions, insertions and/or deletions In another useful aspect, the invention provides a polypeptide that includes a VEGF polypeptide sequence variant having the generalized sequence PCSE X₁X₂X₃ X₄LF VQDPQTCX₅CS CX₆NTDS X₇C X₈A X₉QLELNE X₁₀TC X₁₁CDX₁₂P X₁₃X₁₄ (Seq. ID No. 2), wherein at least one of X₁—X₁₄ corresponds to the position of a non-basic amino acid substitution, the position of an amino acid deletion, or the position of an amino acid insertion of the native VEGF polypeptide sequence PCSERRKHLF VQDPQTCKCS CKNTDSRCKA RQLELNERTC RCDKPRR (Seq. ID No. 1).

In yet another embodiment, the invention provides a VEGF variant having a modified heparin binding function compared to native VEGF while maintaining receptor binding function. In another embodiment, the VEGF variant promotes angiogenesis without increasing leukocyte recruitment or vascular permeability. In another embodiment, VEGF variant comprises a modified Flt-1 binding function and a normal KDR binding function. In another embodiment, the VEGF variant comprises a modified Np-1 binding function and a normal KDR binding function.

Aspects of the invention also provide nucleic acids encoding the VEGF variants.

In another aspect, the invention provides methods for inhibiting the function of the heparin binding domain of VEGF. The invention also provides methods for inhibiting the function of Flt-1 and/or Np-1. In one embodiment, the function of the heparin binding domain of VEGF is inhibited without interfering with the function of the receptor binding domain of VEGF. In another embodiment, the function of Flt-1 is inhibited while the function of KDR is maintained. In another embodiment, the function of Np-1 is inhibited while the function of KDR is maintained.

The VEGF variants of the present invention are useful for promoting angiogenesis without increasing leukocyte recruitment or vascular permeability. The VEGF variants of the present invention are also useful for promoting wound healing, bone repair and bone growth. Compounds capable of binding to the heparin binding domain are capable of inhibiting leukocyte recruitment and inhibiting vascular permeability. The compounds can be useful as anti-inflammatory, anti-vascular permeability, immunosuppressant and anti-hypertension agents.

In another aspect, the invention provides methods of treating a disorder associated with angiogenesis, vascular permeability and inflammation. The invention also provides methods of treating an individual in need of the proliferation of vascular endothelial cells.

In another aspect, the invention provides methods for screening candidate compounds for the capability of promoting angiogenesis without promoting leukocyte recruitment. In one embodiment, the method screens for compounds that inhibit the function of the heparin binding domain without inhibiting the function of the receptor binding domain. In one embodiment, the method screens for compounds that inhibit the function of Flt-1 without inhibiting the function of KDR. In another embodiment, the method screens for compounds that inhibit the function of Np-1 without inhibiting the function of KDR.

In another aspect, the invention provides methods of designing compounds capable of binding to the heparin binding domain. In one embodiment, compounds are designed using SELEX. In another embodiment, compounds are designed using molecular modeling.

In another aspect, the invention provides compounds capable of binding to and/or modifying the function of the heparin binding domain while maintaining the function of the VEGF receptor binding domain.

In another aspect, the invention provides methods of inhibiting VEGF164 induced leukostasis. In one embodiment, the method of inhibiting VEGF164 induced leukostasis comprises administering a soluble heparin binding domain. In one particular embodiment, the soluble heparin binding domain comprises a polypeptide having the sequence of VEGF55.

### BRIEF DESCRIPTION OF THE DRAWINGS

FIG. 1 is a representation of an image of a solution structure of the heparin-binding domain of VEGF165. Amino acid residues R13, R14 and R49 are shown in light-grey. They are critical for the optimum heparin-binding activity as defined by our mutagenesis analysis

FIG. 2 is a representation of two images of a solution structure of the heparin-binding domain of VEGF165. All basic amino acid residues are shown in light-grey. FIG. 2(B) is the view of FIG. 2(A) with the heparin-binding domain of VEGF165 rotated 180 degrees.

FIG. 3 is a representation of images illustrating structural views of the VEGF165 heparin binding domain fragment (VEGF55) and its variants. FIG. 20(A) shows the primary amino acid sequence (residues 1-55) of VEGF165 heparin binding domain. FIG. 20 (B) shows a Ribbon diagram of native VEGF55 (left), a surface topology model (center) and a surface representation as in (centre) rotated by 180 degrees about the vertical axis (right). FIGS. 20(C-L) show the heparin binding domain fragments as ribbon diagrams (left) and surface topology models (right). Lysine and arginine residues selected for mutagenesis are labeled and highlighted (dark regions) and by depiction of their side chains in the ribbon diagram. The numbering of amino acids is based on the primary sequence shown in (A). Individual fragments are labeled by letters and correspond to the following VEGF164 mutants: (C) K30A, (D) R35A/R39A, (E) K30A/R35A/R39A, (F) K30A/R35A/R39A/R49A, (G) K26A, (H) R46A/R49A, (I) R13A/R14A, (J) R14A/R49A, (K) R13A/R14A/R49A, R13A/R14A/R46A/R49A. Figures were generated with Pymol (DeLano Scientific) from the NMR solution structure (Protein Data Bank code: 1KMX).

FIG. 4 is a graph showing the heparin-binding affinities of VEGF variants based on a direct heparin binding assay. The results illustrate the amino acid residues R13, R14 and R49 are critical for the heparin-binding activity of VEGF164 heparin-binding domain.

FIG. 5 is a chart showing the results of Real-Time RT-PCR (Taqman®; Roche Molecular Systems, Inc.) analysis of tissue factor (TF) mRNA up-regulation in HUVE cells by various VEGF variants. The chart illustrates that mutant VEGF variants are functionally active and are comparable to the wild-type VEGF164 in inducing TF expression.

FIG. 6 is a representation of an image of a protein SDS-polyacrylamide gel electrophoresis (PAGE) illustrating that purified VEGF164 mutants proteins are similar to wild-type VEGF164 with respect to mass, glycosylation, and the ability to oligomerize. This PAGE analysis confirms that all the purified VEGF mutant variants are produced as full-length peptides and are processed as the wild-type VEGF164.

FIG. 7 is a chart showing the results of a HUVEC Tissue Factor Assay. The graph illustrates that all VEGF mutants are fully functional in the HUVEC Tissue Factor Assay and are similar to the wild-type VEGF₁₆₄.

FIG. 8 is a representation of two comparisions of the circular dichroism (CD) spectra of Wild Type VEGF164 and Mutants R14/R49A and R13/R14/R49A. FIG. 8(A) shows the CD spectra of WT VEGF164 (solid line) and Mutant R14/R49A (dashed line). FIG. 8(A) shows the CD spectra of WT VEGF164 (solid line) and Mutant R13/R14/R49A (dashed line). The CD analysis of the VEGF variants demonstrated that their mutations did not significantly affect their secondary structures and were comparable to that of the native VEGF164.

FIG. 9 is a graph showing the results of an in vitro VEGF/VEGF-receptor-2 (KDR) plate binding assay. The graph illustrates comparable potencies of inhibiting VEGF164/KDR receptor binding by VEGF164 heparin-binding domain mutants and the wild-type VEGF164, therefore both wild type and mutants VEGF have similar binding affinity toward the KDR receptor. This confirms that the mutagenesis in the heparin-binding domain does not affect the KDR binding site of VEGF164.

FIG. 10 is a graph showing the results of an in vitro VEGF/VEGF-receptor-1 (Flt-1) plate binding assay. The graph illustrates decreased potency of inhibiting VEGF164/Flt-1 binding, and therefore decrease Flt-1 receptor binding affinities by VEGF164 heparin-binding domain mutants R14/R49A and R13/R14/R49A compared to wild-type VEGF164. The results suggest that the heparin-binding domain is involved in the high affinity binding of Flt-1 receptor by VEGF164.

FIG. 11 is a graph showing the results of an in vitro VEGF/neuropilin-1 (Np-1) receptor plate binding assay. The graph illustrates decreased potencies in inhibiting VEGF164/Np-1 binding, and therefore decreased binding affinities to Np-1 receptor by all the VEGF164 heparin-binding domain mutant variants. Furthermore, because mutant K26A has retained much of the heparin-binding activity than either mutant R14/R49A and mutant R13/R14/R49A, the heparin-binding activities of the mutant variants exhibit a positive correlation with their binding affinities toward Flt-1. The results suggest that the heparin-binding domain is involved in the high affinity binding of Np-1 receptor by VEGF164.

FIG. 12 is a chart showing decreased potencies of inhibiting VEGF164/Np-1 binding (increased IC50 values) by the VEGF164 heparin-binding domain mutants when compare to the wild type.

FIG. 13 is a representation showing Scanning Laser Ophthalmascope (SLO) images of rat retinas post injection with VEGF to induce leukostasis. The images illustrate that the heparin-binding domain mutants of VEGF164 have much reduced activities to induce leukostasis in the retina. The results suggest that the heparin-binding domain confers the pro-inflammatory activity of VEGF164.

FIG. 14 is a chart showing the quantified results of the modulation of leukostasis by VEGF164 and its variants. The chart illustrates that the heparin-binding domain mutants are significantly less potent in inducing leukostasis in the retina. The results suggest that the heparin-binding domain is critical for the pro-inflammatory activity of VEGF164 in the retina.

FIG. 15 is a diagram illustrating various VEGF isoforms resulting from an alternatively spliced VEGF mRNA transcript.

FIG. 16(A) is a schematic representation of the polypeptide sequence of human vascular endothelial growth factor (VEGF) corresponding to GenBank Accession No. NP_{—}003367 (SEQ ID NO: 47). The process secretion signal sequence is shown in underlined italics and the mutagenized heparin binding domain sequences are shown in underlined and bolded typeface.

FIG. 16(B) is a schematic representation of the nucleotide sequence of human vascular endothelial growth factor (VEGF) encoding nucleic acid sequence corresponding to GenBank Accession No. NM_{—}003376 (SEQ ID NO: 48).

FIG. 17 is a schematic representation of VEGF exons 7-8 and alanine substitution mutations 1-14.

FIG. 18 is a representation of images of aorta explants captured using epifluorescence microscopy (original magnification, 10×) (left panels). Isolectin B-Immunofluorescence identifies capillary-like microvessels extending from collagen-embedded aortic rings after exposure to PBS, Pichia-derived VEGF120, VEGF164, R14A/R49A, or R13A/R14A/R49A (each 4.4 nM) for 7 days (right panels).

FIG. 19 is a graph showing the quantification of microvascular outgrowth of aorta explants. The total length of traced vessels was determined from the images obtained at day 7 (n=total number of rings from 4 animals, * P<0.001). Bars represent mean±s.e.m.; ANOVA with post hoc Bonferroni test.

FIG. 20 is a representation of an image of a protein SDS-polyacrylamide gel electrophoresis (PAGE) illustrating the heparin-binding characteristics of VEGF wildtype and mutant proteins.

FIG. 21 is a representation of an image of a protein SDS-polyacrylamide gel electrophoresis (PAGE) illustrating the heparin-binding behavior of VEGF164 wildtype and select mutants at physiological salt concentration.

FIG. 22(A) is a graph illustrating the inhibition of VEGF164-induced leukostasis by soluble a soluble HBD. Purified HBD was injected intravitreally into rats either alone or 2 minutes before injecting VEGF164 (2 pmol) in a total volume of 5μl. Leukostasis was evaluated 48 hours later by acridine orange leukocyte fluorography and scanning laser ophthalmoscopy (SLO). Numbers inside bars represent number of eyes analyzed (n). The unpaired Student t test was used for statistical analysis. Differences are considered statistically significant if P<0.05.

FIGS. 22(B-E) is a representationd of fluorescein angiography images of the eye fundus showing adherent leukocytes on retinal microvasculature as white dots. Scale bar=500 μm.

FIG. 23 is a graph illustrating the Suppression of retinal leukostasis by recombinant HBD in mice with oxygen-induced retinopathy. Oxygen-induced retinopathy (OIR) in mice was induced by exposing the animals first to 75% oxygen from P7 to P12 and then to normal air until P14. Injections were performed intravenously at P12 and P13: total goat IgG control (5 mg/kg), goat anti-mouse VEGF neutralizing antibody (5 mg/kg), and purified HBD (2 nmol≅13.3 μg). Adherent leukocytes inside retinal vessels were visualized by perfusion of P14 mouse pups with Con-A lectin and quantified by microscope. Numbers inside the columns represent number of eyes analyzed. The total number of retinal vessels in OIR mice is lower than in non OIR mice due to vessel regression during the hyperoxic phase. P14 mice in the non OIR control group exhibited low levels of leukostasis in the retina.

FIG. 24 is a representation of graphs illustrating the Competitive binding of HBD and VEGF164 to immobilized VEGF receptors. The binding of ¹²⁵I-VEGF165 to immobilized rat neuropilin-1/F_{c} (top panel), mouse VEGFR-1/F_{c} (middle panel), and mouse VEGFR-2/F_{c} (bottom panel) was carried out in the presence of the indicated concentrations of recombinant HBD or VEGF164. Curve fitting and analysis of binding parameters using the one-site competition model were performed with GraphPad software. Specific binding was determined by subtracting the background signal (non-specific signal obtained in the presence of 400 nM VEGF164) from raw signal values. Data points (mean±SEM) are in triplicate and representative of three independent experiments.

FIG. 25 is a graph illustrating the comparison of the binding of VEGF120, VEGF164 and HBD mutants to PAE cells. The figure shows that significantly more VEGF164 bound to PAE cells than VEGF120 or the heparin-binding deficient mutants R14A/R49A and R13A/R14A/R49A (*P<0.05). Data represent the mean±SD of three independent experiments.

FIG. 26 is a representation of immages illustrating binding to the heparan sulfate-rich Bruch's membrane and the inner limiting membrane (ILM) of the eye using an epifluorescence microscope with a digital CCD camera. VEGF164 was capable of binding to both Bruch's membrane and the inner limiting membrane (arrows) in the retina. No labeling of either Bruch's or inner limiting membrane (asterisks) was observed in sections treated with VEGF120. The scale bar represents 10 μm.

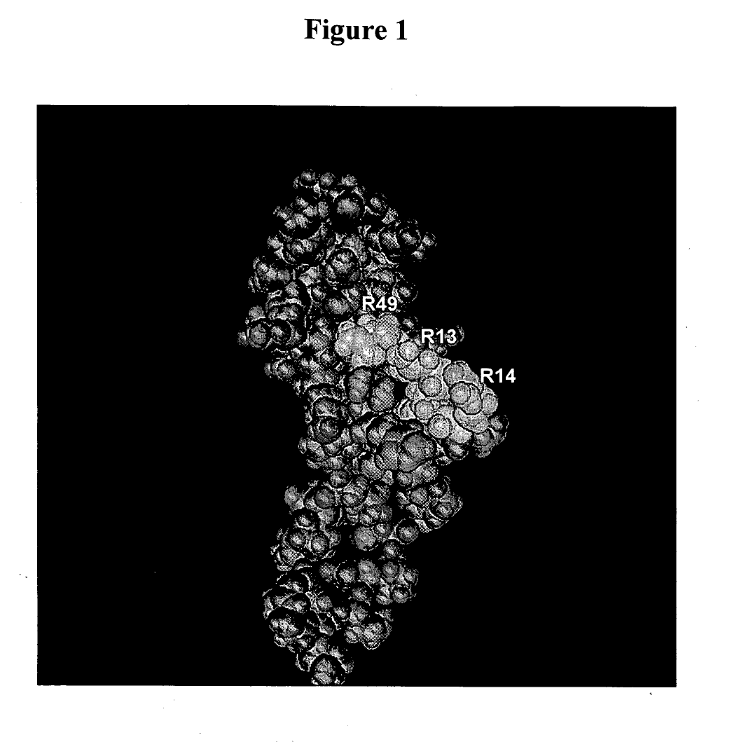

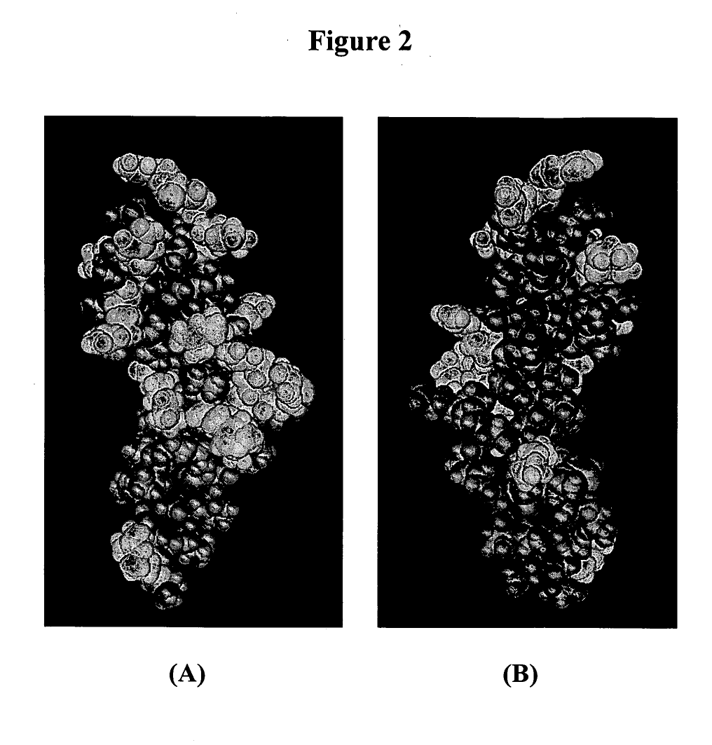

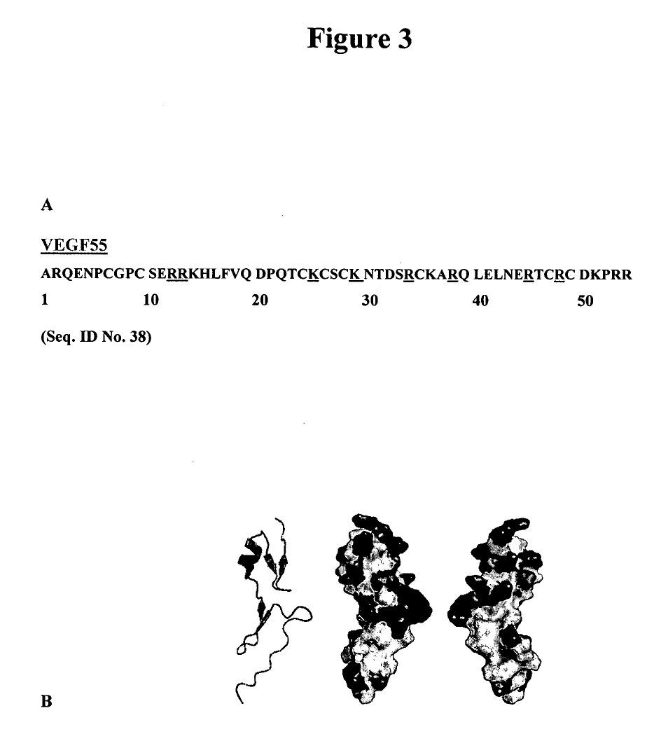

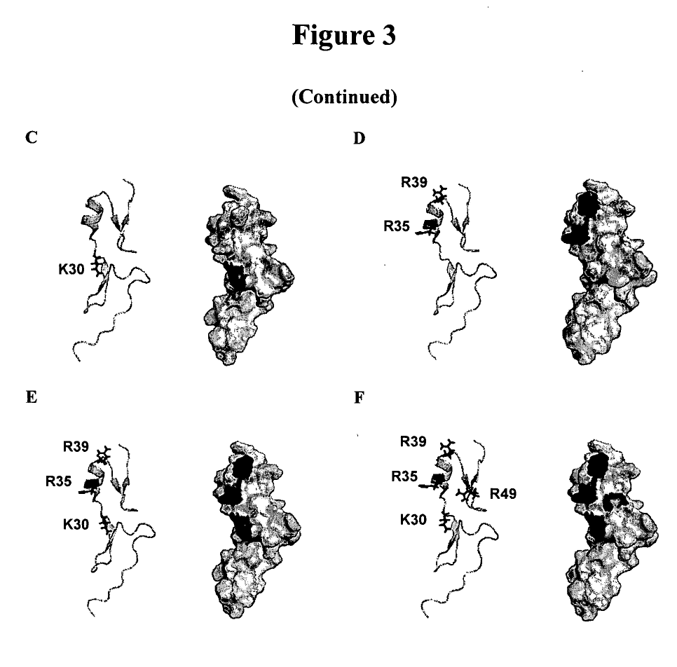

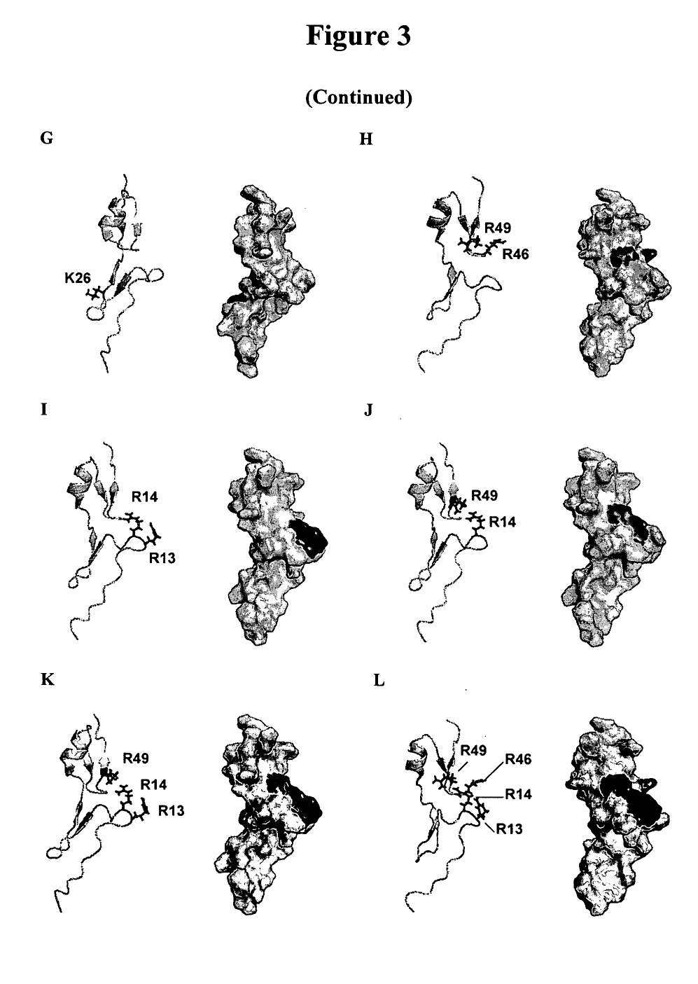

### DETAILED DESCRIPTION OF THE INVENTION

Applicants have discovered that a functional heparin-binding domain of VEGF164 is required for pathological neovascularization and its pro-inflammation activity. Previous studies have demonstrated that ischemia-induced retinal neovascularization is caused in part by a local inflammatory response. Since the VEGF164 isoform has an enhanced capacity to trigger pro-inflammatory events, Applicants have characterized its role in inflammation and pathological neovascularization using VEGF164-deficient (VEGF120/188) mice. VEGF164 protein mutants in which the heparin-binding domain (HBD) is inactivated through point-directed mutagenesis was used to define the pro-inflammatory domain of the VEGF164 protein molecule.

The results show that under normal developmental conditions the retina vasculatures of the VEGF120/188 mice developed normally and are comparable to that of the age-matched wild-type littermates. The results also show that the VEGF164 protein is not required for normal vascular development in the retina, and that the combination of the VEGF120 and VEGF188 isoforms are sufficient to drive physiological retinal vessel growth.

In a retinopathy of prematurity (ROP) model after 5 days of hyperoxia, there is no difference in the vascular obliteration between VEGF120/188 and wild-type mice. The data show that the retinal vasculatures of these VEGF164-deficient mice are susceptible to vascular regression due to down-regulation of local VEGF levels in the retina. Pathological neovascularization following return to normoxic conditions (relative hypoxia) was suppressed by over 90% in the VEGF120/188 mice as compared to wild type littermates. In contrast, no suppression of physiological revascularization was observed in the VEGF120/188 retinas in the ROP model. The lack of VEGF164 protein also resulted in a significant decrease of inflammatory response in the VEGF1 20/188 retinal vasculature in the ROP model.

Additionally, using a skin delayed type hypersensitivity (DTH) model, the lack of VEGF164 protein also significantly reduces the inflammation in the VEGF120/188 mice suggesting that the VEGF164 protein is associated with pathological angiogenesis and that its pro-inflammatory nature is confirmed both in the eye and skin. It has therefore been discovered that the VEGF164 protein isoform is likely to be pro-inflammatory in all tissue types. The pro-inflammatory nature of the VEGF164 protein isoform is conferred by its heparin binding domain because the VEGF120 protein isoform is shown to be not associated with pro-inflammatory events.

The administration of non-heparin-binding VEGF164 protein mutants, which contain point mutations in arginine residues 13, 14 and 49 of the heparin binding domain, in the vitreous of rat failed to recruit leukocytes, whereas significant leukostasis was induced by the wild-type VEGF164 protein injection. The data show that a functional heparin binding domain is required for the pro-inflammatory and pathological nature of the VEGF164 protein isoform. Thus the heparin binding domain of VEGF164 is responsible for its unique biological activity and pathological nature among the different VEGF isoform. These results suggest an electrostatic and protein sequence-specific component in the VEGF-heparin interaction, which may confer unique biological functions to VEGF.

The VEGF variant compositions and methods of the present invention are useful for treating cardiovascular diseases or conditions requiring therapeutic neovascularization. Such cardiovascular diseases or conditions include, but are not limited to, myocardial ischemia, coronary artery disease and peripheral arterial disease.

The VEGF variant compositions and methods of the present invention are also useful for promoting normal embryonic development (vasculogenesis), wound healing, female reproductive function, hematopoietic stem cell (HSC) mobilization from the bone marrow, monocyte chemoattraction and osteoblast-mediated bone formation.

The VEGF variant compositions and methods of the present invention are useful for treating neuron disorders. In particular, the VEGF variant compositions and methods of the present invention are useful for promoting neuroprotection.

The VEGF variant compositions and methods of the present invention are useful for treating disorders such as amyotrophic lateral sclerosis (ALS, Lou Gehrig's disease) and ALS-like diseases, which are characterized by defective VEGF survival signals to neurons.

The VEGF variant compositions and methods of the present invention are also useful for protecting the neuronal cells in the retina, in particular, during hypoxia in ischemic eye diseases.

The VEGF variant compositions and methods of the present invention are also useful for protecting motoneurons, preventing motor neuron degeneration and prolonging their survival.

The VEGF variant compositions and methods of the present invention are also useful for stimulating neural stem cells.

VEGF165 has been shown to stimulate survival of neurons or inhibit death of neurons by, for example, binding to Neuropilin-1, a receptor known to bind semaphoring 3A, which is implicated in axon retraction and neuronal death and VEGF Receptor-2 (Carmeleit et al., WO 01/76620, which is incorporated herein by reference in its entirety). VEGF stimulates axonal outgrowth, improves the survival of superior cervical and dorsal route ganglion neurons, and enhances the survival of mesencephalic neurons. VEGF can rescue HN33 hippocampal cells from apopotosis.

The VEGF variant compositions and methods of the present invention are also useful for promoting angiogenesis or therapeutic neovascularization without the negative effects of inflammation or vascular permeability. The VEGF variant compositions and methods of the present invention are useful for treating any subject in need of developing new blood vessels from existing endothelium. New blood vessels may be needed in any tissue having insufficient blood flow, such as for example, hypoxic or ischemic tissue.

In one aspect, the invention provides novel VEGF variants. The VEGF variants comprise a polypeptide having a modified heparin-binding domain. In one embodiment, the heparin binding domain is modified by substituting basic amino acid residues with neutral amino acid residues or acidic amino acid residues. In another embodiment, the heparin binding domain is modified by inserting a non-basic amino acids adjacent to a basic amino acids. In another embodiment, the heparin binding domain is modified by deleteing basic amino acids. The invention also provides nucleic acids encoding the VEGF variants.

In one embodiment, a VEGF variant has a modified heparin binding function compared to native VEGF while maintaining receptor binding function. In another embodiment, the VEGF variant promotes angiogenesis without increasing leukocyte recruitment or vascular permeability. In another embodiment, VEGF variant comprises a modified Flt-1 binding function and a normal KDR binding function. In another embodiment, the VEGF variant comprises a modified Np-1 binding function and a normal KDR binding function.

In yet another embodiment, the native VEGF polypeptide sequence is PCSERRKHLF VQDPQTCKCS CKNTDSRCKA RQLELNERTC RCDKPRR (Seq. ID No. 1). In further embodiments, the VEGF polypeptide sequence variant has the sequence PCSEX₁X₂KHLF VQDPQTCKCS CKNTDSRCKA RQLELNERTC X₃CDKPRR (Seq. ID No.28), and X₁, X₂, and X₃ are R or a non-basic amino acid, but at least one of X₁, X₂, and X₃ is a non-basic amino acid. In certain particularly useful embodiments, the non-basic amino acid is alanine. In other embodiments, the VEGF polypeptide sequence variant has the sequence PCSERAKHLF VQDPQTCKCS CKNTDSRCKA RQLELNERTC ACDKPRR (Seq. ID No. 3). In still other embodiments, the VEGF polypeptide sequence variant has the sequence PCSEAAKHLF VQDPQTCKCS CKNTDSRCKA RQLELNERTC ACDKPRR (Seq. ID No. 4). In further embodiments, the polypeptide has the sequence PCSEX₁X₂KHLF VQDPQTCKCS CKNTDSRCKA RQLELNERTC X₃CDKPRR (Seq. ID No. 28) and X₁ and X₂, are R, and X₃ is a non-basic amino acid. In particular embodiments, the non-basic amino acid that is substituted is A, N, D, C, Q, E, I, L, M, S, T, or V. In a particular embodiment, X₁ and X₂, are R, and X₃ is A. In another particular embodiment, X₁, X₂, and X₃ are A.

In still other embodiments, the polypeptide has the sequence PCSEX₁X₂KHLF VQDPQTCKCS CKNTDSRCKA RQLELNERTC X₃CDKPRR (Seq. ID No. 28) and X₁ and X₃, are R, and X₂ is a non-basic amino acid. In particular embodiments, non-basic amino acid that is substituted is A, N, D, C, Q, E, I, L, M, S, T, or V. In certain embodiments, X₁ and X₂, are R, and X₃ is A. In further embodiments, X₂ and X₃, are R, and X₁ is a non-basic amino acid. In particular embodiments, the non-basic amino acid that is substituted is A, N, D, C, Q, E, I, L, M, S, T, or V. In a particular embodiment, X₂ and X₃, are R, and X₁ is A. In another particular embodiment, X₁, X₂, and X₃ are A.

In particular embodiments, the VEGF variant comprises a polypeptide having the sequence selected from the group consisting of: PCSERAKHLF VQDPQTCKCS CKNTDSRCKA RQLELNERTC ACDKPRR (Seq. ID No. 3); PCSEAAKHLF VQDPQTCKCS CKNTDSRCKA RQLELNERTC ACDKPRR (Seq. ID No. 4); PCSERRKHLF VQDPQTCKCS CANTDSACKA AQLELNERTC RCDKPRR (Seq. ID No. 5); PCSERRKHLF VQDPQTCKCS CKNTDSACKA AQLELNERTC RCDKPRR (Seq. ID No. 6); PCSERRKHLF VQDPQTCKCS CANTDSRCKA RQLELNERTC RCDKPRR (Seq. ID No. 7); PCSERRKHLF VQDPQTCKCS CANTDSACKA AQLELNERTC ACDKPRR (Seq. ID No. 8); PCSERRKHLF VQDPQTCKCS CANTDSRCKA RQLELNERTC RCDKPRR (Seq. ID No. 9); PCSERRKHLF VQDPQTCKCS CKNTDSRCKA RQLELNEATC ACDKPRR (Seq. ID No. 10); PCSEAAKHLF VQDPQTCKCS CKNTDSRCKA RQLELNERTC RCDKPRR (Seq. ID No. 11); and PCSEAAKHLF VQDPQTCKCS CKNTDSRCKA RQLELNEATC ACDKPRR (Seq. ID No. 12).

In other particular embodiments, the VEGF variant comprises a polypeptide having the sequence selected from the group consisting of: ARQENPCGPC SERAKHLFVQ DPQTCKCSCK NTDSRCKARQ LELNERTCAC DKPRR (Seq. ID No. 13); ARQENPCGPC SEAAKHLFVQ DPQTCKCSCK NTDSRCKARQ LELNERTCAC DKPRR (Seq. ID No. 14); ARQENPCGPC SERRKHLFVQ DPQTCKCSCA NTDSACKAAQ LELNERTCRC DKPRR (Seq. ID No. 15); ARQENPCGPC SERRKHLFVQ DPQTCKCSCK NTDSACKAAQ LELNERTCRC DKPRR (Seq. ID No. 16); ARQENPCGPC SERRKHLFVQ DPQTCKCSCA NTDSRCKARQ LELNERTCRC DKPRR (Seq. ID No. 17); ARQENPCGPC SERRKHLFVQ DPQTCKCSCA NTDSACKAAQ LELNERTCAC DKPRR (Seq. ID No. 18); ARQENPCGPC SERRKHLFVQ DPQTCKCSCA NTDSRCKARQ LELNERTCRC DKPRR (Seq. ID No. 19); ARQENPCGPC SERRKHLFVQ DPQTCKCSCK NTDSRCKARQ LELNEATCAC DKPRR (Seq. ID No. 20); ARQENPCGPC SEAAKHLFVQ DPQTCKCSCK NTDSRCKARQ LELNERTCRC DKPRR (Seq. ID No. 21); and ARQENPCGPC SEAAKHLFVQ DPQTCKCSCK NTDSRCKARQ LELNEATCAC DKPRR (Seq. ID No. 22).

In certain embodiments, the polypeptide comprising the VEGF polypeptide sequence variant has the sequence APMA EGGGQNHHEV VKFMDVYQRS YCHPIETLVD IFQEYPDEIE YIFKPSCVPL MRCGGCCNDE GLECVPTEES NITMQIMRIK PHQGQHIGEM SFLQHNKCEC RPKKDRARQE NPCGPC SERAKHLFVQ DPQTCKCSCK NTDSRCKARQ LELNERTCAC DKPRR (Seq. ID No. 23). In other embodiments, the polypeptide has the sequence APMA EGGGQNHHEV VKFMDVYQRS YCHPIETLVD IFQEYPDEIE YIFKPSCVPL MRCGGCCNDE GLECVPTEES NITMQIMRIK PHQGQHIGEM SFLQHNKCEC RPKKDRARQE NPCGPC SEAAKHLFVQ DPQTCKCSCK NTDSRCKARQ LELNERTCAC DKPRR (Seq. ID No. 24). In still other embodiments, the polypeptide has the sequence APMA EGGGQNHHEV VKFMDVYQRS YCHPIETLVD IFQEYPDEIE YIFKPSCVPL MRCGGCCNDE GLECVPTEES NITMQIMRIK PHQGQHIGEM SFLQHNKCEC RPKKDRARQE NPCGPC SERRKHLFVQ DPQTCKCSCK NTDSRCKARQ LELNERTCAC DKPRR (Seq. ID No.25). In a further embodiment, the polypeptide has the sequence APMA EGGGQNHHEV VKFMDVYQRS YCHPIETLVD IFQEYPDEIE YIFKPSCVPL MRCGGCCNDE GLECVPTEES NITMQIMRIK PHQGQHIGEM SFLQHNKCEC RPKKDRARQE NPCGPC SERAKHLFVQ DPQTCKCSCK NTDSRCKARQ LELNERTCRC DKPRR (Seq. ID No.26). In still further embodiments, the polypeptide has the sequence: APMA EGGGQNHHEV VKFMDVYQRS YCHPIETLVD IFQEYPDEIE YIFKPSCVPL MRCGGCCNDE GLECVPTEES NITMQIMRIK PHQGQHIGEM SFLQHNKCEC RPKKDRARQE NPCGPC SEARKHLFVQ DPQTCKCSCK NTDSRCKARQ LELNERTCRC DKPRR (Seq. ID No. 27).

In further particularly useful embodiments, the VEGF polypeptide sequence variant with reduced pro-inflammatory activity induces less leukostasis when administered in the retina than does the corresponding native VEGF polypeptide sequence.

In another aspect, the invention provides polypeptides that include alterations of a native VEGF polypeptide sequence that reduces neuropilin-1 receptor binding activity, while substantially maintaining the affinity for VEGR-2 (FLK-1/KDR). In certain embodiments, the native VEGF polypeptide sequence is human VEGF165. In other embodiments, the native VEGF polypeptide sequence is human VEGF189. In further embodiments, the native VEGF polypeptide sequence is human VEGF206. In still other embodiments, the native VEGF polypeptide sequence is mouse VEGF164. In still further embodiments, the native VEGF polypeptide sequence is a VEGF isoform of a mammal such as a human, a mouse, a rat, a monkey, a cow, a pig, a sheep, a dog, a cat, or a rabbit.

In yet another embodiment, the native VEGF polypeptide sequence is PCSERRKHLF VQDPQTCKCS CKNTDSRCKA RQLELNERTC RCDKPRR (Seq. ID No. 1). In further embodiments, the VEGF polypeptide sequence variant has the sequence PCSEX₁X₂KHLF VQDPQTCKCS CKNTDSRCKA RQLELNERTC X₃CDKPRR (Seq. ID No. 28), and X₁, X₂, and X₃ are R or a non-basic amino acid, but at least one of X₁, X₂, and X₃ is a non-basic amino acid. In certain particularly useful embodiments, the non-basic amino acid is alanine. In other embodiments, the VEGF polypeptide sequence variant has the sequence PCSERAKHLF VQDPQTCKCS CKNTDSRCKA RQLELNERTC ACDKPRR (Seq. ID No.3). In still other embodiments, the VEGF polypeptide sequence variant has the sequence PCSEAAKHLF VQDPQTCKCS CKNTDSRCKA RQLELNERTC ACDKPRR (Seq. ID No. 4). In further embodiments, the polypeptide has the sequence PCSEX₁X₂KHLF VQDPQTCKCS CKNTDSRCKA RQLELNERTC X₃CDKPRR (Seq. ID No. 28) and X₁ and X₂, are R, and X₃ is a non-basic amino acid. In particular embodiments, the non-basic amino acid that is substituted is A, N, D, C, Q, E, I, L, M, S, T, or V. In a particular embodiment, X₁ and X₂, are R, and X₃ is A. In another particular embodiment, X₁, X₂, and X₃ are A.

In still other embodiments, the polypeptide has the sequence PCSEX₁X₂KHLF VQDPQTCKCS CKNTDSRCKA RQLELNERTC X₃CDKPRR (Seq. ID No. 28) and X₁ and X₃, are R, and X₂ is a non-basic amino acid. In particular embodiments, non-basic amino acid that is substituted is A, N, D, C, Q, E, I, L, M, S, T, or V. In certain embodiments, X₁ and X₂, are R, and X₃ is A. In further embodiments, X₂ and X₃, are R, and X₁ is a non-basic amino acid. In particular embodiments, the non-basic amino acid that is substituted is A, N, D, C, Q, E, I, L, M, S, T, or V. In a particular embodiment, X₂ and X₃, are R, and X₁ is A. In another particular embodiment, X₁, X₂, and X₃ are A.

In certain further embodiments, the polypeptide comprising the VEGF polypeptide sequence variant has the sequence APMA EGGGQNHHEV VKFMDVYQRS YCHPIETLVD IFQEYPDEIE YIFKPSCVPL MRCGGCCNDE GLECVPTEES NITMQIMRIK PHQGQHIGEM SFLQHNKCEC RPKKDRARQE NPCGPC SERAKHLFVQ DPQTCKCSCK NTDSRCKARQ LELNERTCAC DKPRR (Seq. ID No. 23). In other embodiments, the polypeptide has the sequence APMA EGGGQNHHEV VKFMDVYQRS YCHPIETLVD IFQEYPDEIE YIFKPSCVPL MRCGGCCNDE GLECVPTEES NITMQIMRIK PHQGQHIGEM SFLQHNKCEC RPKKDRARQE NPCGPC SEAAKHLFVQ DPQTCKCSCK NTDSRCKARQ LELNERTCAC DKPRR (Seq. ID No. 24). In still other embodiments, the polypeptide has the sequence APMA EGGGQNHHEV VKFMDVYQRS YCHPIETLVD IFQEYPDEIE YIFKPSCVPL MRCGGCCNDE GLECVPTEES NITMQIMRIK PHQGQHIGEM SFLQHNKCEC RPKKDRARQE NPCGPC SERRKHLFVQ DPQTCKCSCK NTDSRCKARQ LELNERTCAC DKPRR (Seq. ID No. 25). In a further embodiment, the polypeptide has the sequence APMA EGGGQNHHEV VKFMDVYQRS YCHPIETLVD IFQEYPDEIE YIFKPSCVPL MRCGGCCNDE GLECVPTEES NITMQIMRIK PHQGQHIGEM SFLQHNKCEC RPKKDRARQE NPCGPC SERAKHLFVQ DPQTCKCSCK NTDSRCKARQ LELNERTCRC DKPRR (Seq. ID No. 26). In still further embodiments, the polypeptide has the sequence: APMA EGGGQNHHEV VKFMDVYQRS YCHPIETLVD IFQEYPDEIE YIFKPSCVPL MRCGGCCNDE GLECVPTEES NITMQIMRIK PHQGQHIGEM SFLQHNKCEC RPKKDRARQE NPCGPC SEARKHLFVQ DPQTCKCSCK NTDSRCKARQ LELNERTCRC DKPRR (Seq. ID No. 27).

In further particularly useful embodiments, the VEGF polypeptide sequence variant with reduced pro-inflammatory activity induces less leukostasis when administered to the retina than does the corresponding native VEGF polypeptide sequence.

In a particularly useful aspect, the invention provides a polypeptide that includes a VEGF polypeptide sequence variant that has a reduced pro-inflammatory activity in which the VEGF polypeptide variant has one or more alterations of a native VEGF polypeptide sequence. In particularly useful embodiments, the native VEGF polypeptide sequence is PCSERRKHLF VQDPQTCKCS CKNTDSRCKA RQLELNERTC RCDKPRR (Seq. ID No.1) and the alteration is one or more amino acid substitutions, amino acid insertions or amino acid deletions, or a combination thereof.

In yet another aspect, the invention provides a polypeptide that includes a VEGF polypeptide sequence variant having one or more amino acid substitutions, amino acid insertions and/or amino acid deletions of the native VEGF polypeptide sequence PCSERRKHLF VQDPQTCKCS CKNTDSRCKA RQLELNERTC RCDKPRR (Seq. ID No. 1). In certain embodiments, the polypeptide includes one or more substitutions of a basic amino acid of the native VEGF polypeptide sequence with a non-basic amino acid. In other embodiments, the polypeptide includes one or more deletions of a basic amino acid of the native VEGF polypeptide sequence. In other embodiments, the polypeptide includes one or more insertions of a non-basic amino acid adjacent to a basic amino acid of the native VEGF polypeptide sequence. In other embodiments, the polypeptide includes a combination of substitutions, insertions and/or deletions In another useful aspect, the invention provides a polypeptide that includes a VEGF polypeptide sequence variant having the generalized sequence PCSE X₁X₂X₃ X₄LF VQDPQTCX₅CS CX₆NTDS X₇C X₈A X₉QLELNE X₁₀TC X₁₁CDX₁₂P X₁₃X₁₄ (Seq. ID No. 2), wherein at least one of X₁—X₁₄ is a non-basic amino acid substitution of the native VEGF polypeptide sequence PCSERRKHLF VQDPQTCKCS CKNTDSRCKA RQLELNERTC RCDKPRR (Seq. ID No. 1).

In a further useful aspect, the invention provides a polypeptide that includes a VEGF polypeptide sequence variant having the sequence PCSE X₁X₂X₃ X₄LF VQDPQTCX₅CS CX₆NTDS X₇C X₈A XgQLELNE X₁₀TC X₁₁CDX₁₂P X₁₃X₁₄ (Seq. ID No. 2), wherein at least one of X₁, X₂, and X₅—X₁₁ is a non-basic amino acid substitution of the native VEGF polypeptide sequence PCSERRKHLF VQDPQTCKCS CKNTDSRCKA RQLELNERTC RCDKPRR (Seq. ID No.1). In certain embodiments, the non-basic amino acid substitution is with an amino such as A, N, D, C, Q, E, I, L, M, S, T or V. In a particularly useful embodiment, the non-basic amino acid substitution is an A.

In particular embodiments, the polypeptide has the sequence APMA EGGGQNHHEV VKFMDVYQRS YCHPIETLVD IFQEYPDEIE YIFKPSCVPL MRCGGCCNDE GLECVPTEES NITMQIMRIK PHQGQHIGEM SFLQHNKCEC RPKKDRARQE NPCGPC SERAKHLFVQ DPQTCKCSCK NTDSRCKARQ LELNERTCAC DKPRR (Seq. ID No. 23). In other embodiments, the polypeptide has the sequence APMA EGGGQNHHEV VKFMDVYQRS YCHPIETLVD IFQEYPDEIE YIFKPSCVPL MRCGGCCNDE GLECVPTEES NITMQIMRIK PHQGQHIGEM SFLQHNKCEC RPKKDRARQE NPCGPC SEAAKHLFVQ DPQTCKCSCK NTDSRCKARQ LELNERTCAC DKPRR (Seq. ID No. 24). In still other embodiments, the polypeptide has the sequence APMA EGGGQNHHEV VKFMDVYQRS YCHPIETLVD IFQEYPDEIE YIFKPSCVPL MRCGGCCNDE GLECVPTEES NITMQIMRIK PHQGQHIGEM SFLQHNKCEC RPKKDRARQE NPCGPC SERRKHLFVQ DPQTCKCSCK NTDSRCKARQ LELNERTCAC DKPRR (Seq. ID No. 25). In yet other embodiments, the polypeptide has the sequence APMA EGGGQNHHEV VKFMDVYQRS YCHPIETLVD IFQEYPDEIE YIFKPSCVPL MRCGGCCNDE GLECVPTEES NITMQIMRIK PHQGQHIGEM SFLQHNKCEC RPKKDRARQE NPCGPC SERAKHLFVQ DPQTCKCSCK NTDSRCKARQ LELNERTCRC DKPRR (Seq. ID No. 26). In still further embodiments, the polypeptide has the sequence APMA EGGGQNHHEV VKFMDVYQRS YCHPIETLVD IFQEYPDEIE YIFKPSCVPL MRCGGCCNDE GLECVPTEES NITMQIMRIK PHQGQHIGEM SFLQHNKCEC RPKKDRARQE NPCGPC SEARKHLFVQ DPQTCKCSCK NTDSRCKARQ LELNERTCRC DKPRR (Seq. ID No. 27).

In a further aspect, the invention provides a VEGF polypeptide sequence variant that includes the sequence PCSE X₁X₂X₃ X₄LF VQDPQTCX₅CS CX₆NTDS X₇C X₈A X₉QLELNE X₁₀TC X₁₁CDX₁₂P X₁₃X₁₄ (Seq. ID No. 2), wherein and at least one of X₁—X₁₄ corresponds to the position of an amino acid deletion of the native VEGF polypeptide sequence PCSERRKHLF VQDPQTCKCS CKNTDSRCKA RQLELNERTC RCDKPRR (Seq. ID No. 1).

In still another useful aspect, the invention provides a VEGF polypeptide sequence variant that includes the sequence PCSE X₁X₂X₃ X₄LF VQDPQTCX₅CS CX₆NTDS X₇C X₈A X₉QLELNE X₁₀TC X₁₁CDX₁₂P X₁₃X₁₄ (Seq. ID No. 2), wherein at least one of X₁, X₂, and X₅—X₁₁ corresponds to the position of an amino acid deletion of the native VEGF polypeptide sequence PCSERRKHLF VQDPQTCKCS CKNTDSRCKA RQLELNERTC RCDKPRR (Seq. ID No. 1). In certain embodiments of this aspect, the polypeptide has the sequence: APMA EGGGQNHHEV VKFMDVYQRS YCHPIETLVD IFQEYPDEIE YIFKPSCVPL MRCGGCCNDE GLECVPTEES NITMQIMRIK PHQGQHIGEM SFLQHNKCEC RPKKDRARQE NPCGPC SERKHLFVQ DPQTCKCSCK NTDSRCKARQ LELNERTCC DKPRR (Seq. ID No. 29). In other embodiments, the invention provides a polypeptide having the sequence APMA EGGGQNHHEV VKFMDVYQRS YCHPIETLVD IFQEYPDEIE YIFKPSCVPL MRCGGCCNDE GLECVPTEES NITMQIMRIK PHQGQHIGEM SFLQHNKCEC RPKKDRARQE NPCGPC SEKHLFVQ DPQTCKCSCK NTDSRCKARQ LELNERTCC DKPRR (Seq. ID No. 30). In still other embodiments, the invention provides a polypeptide having the sequence APMA EGGGQNHHEV VKFMDVYQRS YCHPIETLVD IFQEYPDEIE YIFKPSCVPL MRCGGCCNDE GLECVPTEES NITMQIMRIK PHQGQHIGEM SFLQHNKCEC RPKKDRARQE NPCGPC SERRKHLFVQ DPQTCKCSCK NTDSRCKARQ LELNERTCC DKPRR (Seq. ID No. 31). In further embodiments, the polypeptide has the sequence APMA EGGGQNHHEV VKFMDVYQRS YCHPIETLVD IFQEYPDEIE YIFKPSCVPL MRCGGCCNDE GLECVPTEES NITMQIMRIK PHQGQHIGEM SFLQHNKCEC RPKKDRARQE NPCGPC SERKHLFVQ DPQTCKCSCK NTDSRCKARQ LELNERTCRC DKPRR (Seq. ID No. 32).

In a further aspect, the invention provides a polypeptide that includes a VEGF polypeptide sequence variant having the generalized sequence PCSE X₁X₂X₃ X₄LF VQDPQTCX₅CS CX6NTDS X₇C X₈A X₉QLELNE X₁₀TC X₁₁CDX₁₂P X₁₃X₁₄ (Seq. ID No. 2), wherein at least one of X₁—X₁₄ corresponds to the position of an amino acid insertion of the native VEGF polypeptide sequence PCSERRKHLF VQDPQTCKCS CKNTDSRCKA RQLELNERTC RCDKPRR (Seq. ID No.1).

In another particularly useful aspect, the invention provides a polypeptide that includes a VEGF polypeptide sequence variant that has the general sequence PCSE X₁X₂X₃ X₄LF VQDPQTCX₅CS CX₆NTDS X₇C X₈A X₉QLELNE X₁₀TC X₁₁CDX₁₂P X₁₃X₁₄ (Seq. ID No. 2), wherein at least one of X₁, X₂, and X₅—X₁₁ corresponds to the position of an amino acid insertion of the native VEGF polypeptide sequence PCSERRKHLF VQDPQTCKCS CKNTDSRCKA RQLELNERTC RCDKPRR (Seq. ID No.1). Insertions may be made adjacent to either side of the native amino acid.

In certain embodiments, the polypeptide has the sequence APMA EGGGQNHHEV VKFMDVYQRS YCHPIETLVD IFQEYPDEIE YIFKPSCVPL MRCGGCCNDE GLECVPTEES NITMQIMRIK PHQGQHIGEM SFLQHNKCEC RPKKDRARQE NPCGPC SERARKHLFVQ DPQTCKCSCK NTDSRCKARQ LELNERTCARC DKPRR (Seq. ID No.33). In other embodiments, the polypeptide has the sequence APMA EGGGQNHHEV VKFMDVYQRS YCHPIETLVD IFQEYPDEIE YIFKPSCVPL MRCGGCCNDE GLECVPTEES NITMQIMRIK PHQGQHIGEM SFLQHNKCEC RPKKDRARQE NPCGPC SEARARKHLFVQ DPQTCKCSCK NTDSRCKARQ LELNERTCARC DKPRR (Seq. ID No.34). In still other embodiments, the polypeptide has the sequence APMA EGGGQNHHEV VKFMDVYQRS YCHPIETLVD IFQEYPDEIE YIFKPSCVPL MRCGGCCNDE GLECVPTEES NITMQIMRIK PHQGQHIGEM SFLQHNKCEC RPKKDRARQE NPCGPC SERRKHLFVQ DPQTCKCSCK NTDSRCKARQ LELNERTCARC DKPRR (Seq. ID No.35). In further embodiments, the polypeptide has the sequence APMA EGGGQNHHEV VKFMDVYQRS YCHPIETLVD IFQEYPDEIE YIFKPSCVPL MRCGGCCNDE GLECVPTEES NITMQIMRIK PHQGQHIGEM SFLQHNKCEC RPKKDRARQE NPCGPC SERARKHLFVQ DPQTCKCSCK NTDSRCKARQ LELNERTCRC DKPRR (Seq. ID No. 36). In still other embodiments, the polypeptide has the sequence APMA EGGGQNHHEV VKFMDVYQRS YCHPIETLVD IFQEYPDEIE YIFKPSCVPL MRCGGCCNDE GLECVPTEES NITMQIMRIK PHQGQHIGEM SFLQHNKCEC RPKKDRARQE NPCGPC SEARRKHLFVQ DPQTCKCSCK NTDSRCKARQ LELNERTCRC DKPRR (Seq. ID No.37).

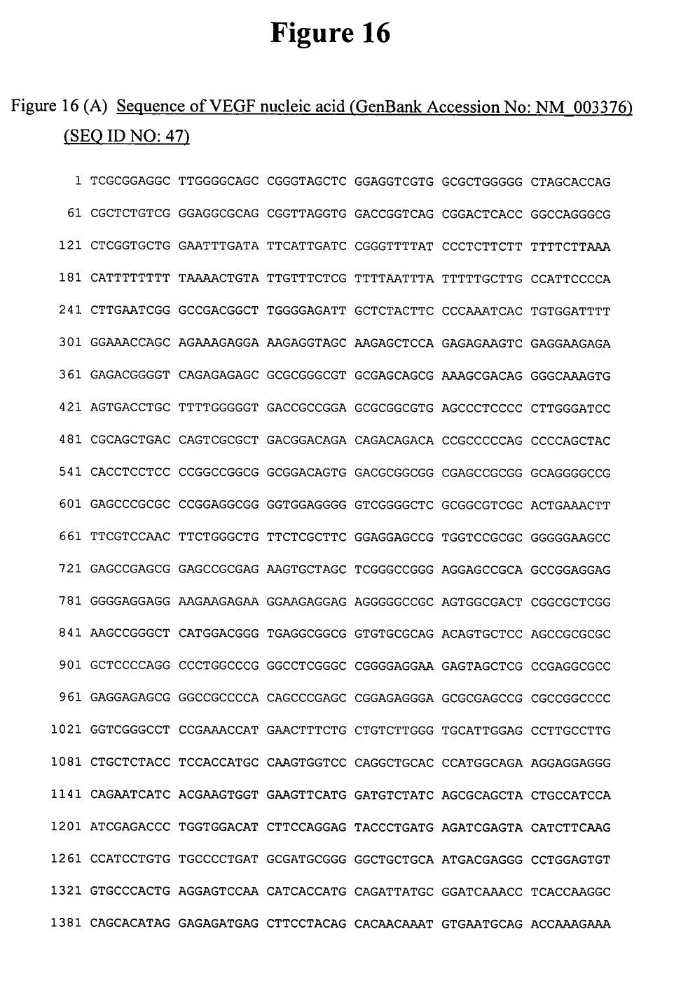

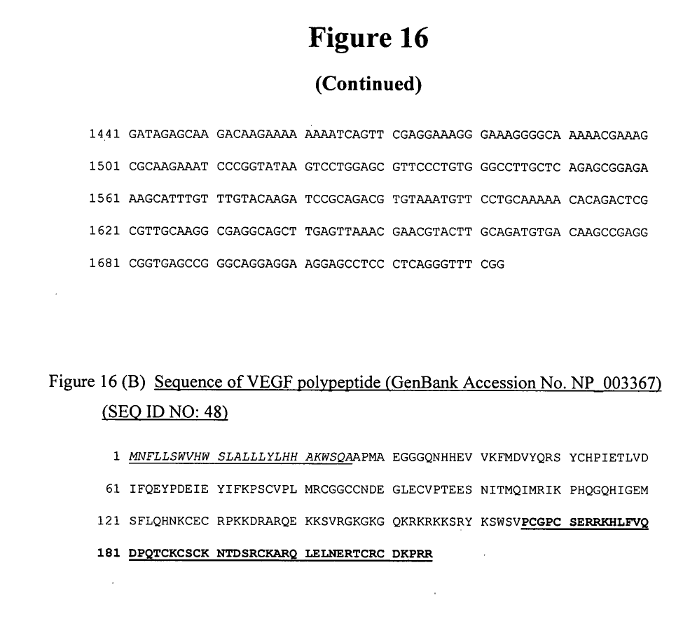

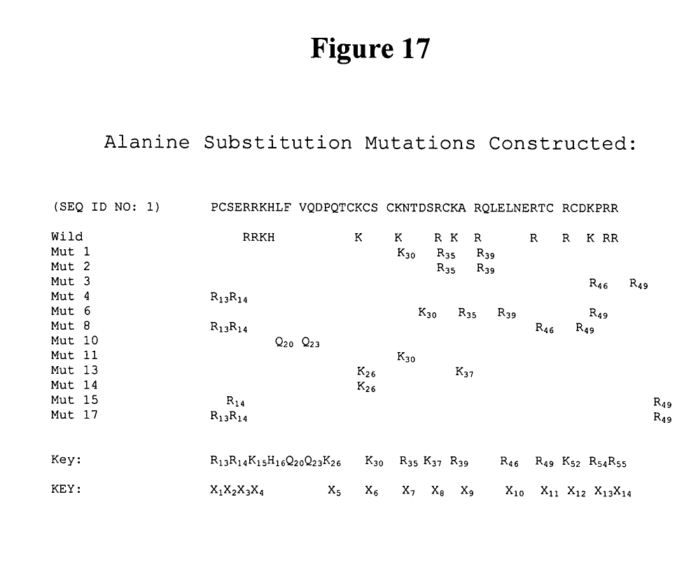

In certain particularly useful embodiments of any of the above aspects of the inventions, polypeptide includes a VEGF polyeptide sequence variant that is encoded by a nucleic acid that hybridizes under stringent conditions to a nucleic acid that encodes a native mammalian VEGF cDNA. In certain embodiments, the the native mammalian VEGF cDNA to which the nucleic acid encoding the variant hybridizes is the human VEGF cDNA of GenBank Accession No. NM_{—}003376 (See FIG. 16).

In another aspect, the invention provides a method of treating a disease or disorder using a VEGF polypeptide with reduced inflammatory side effects by administering any of the polypeptides of the above aspects of the invention.

In a particularly useful aspect, the invention provides a method of treating a disease or condition with a VEGF polypeptide with reduced inflammatory side effects by administering a VEGF polypeptide sequence variant having one or more alterations of a native VEGF polypeptide sequence that reduces heparin binding affinity, while substantially maintaining the affinity for VEGR-2 (FLK-1/KDR).

In certain embodiments of this method of the invention, the VEGF polypeptide sequence variant has one or more amino acid substitutions of a basic amino acid residue of the native VEGF polypeptide sequence PCSERRKHLF VQDPQTCKCS CKNTDSRCKA RQLELNERTC RCDKPRR (Seq. ID No. 1). In certain embodiments, the VEGF polypeptide sequence variant has the sequence PCSEX₁X₂KHLF VQDPQTCKCS CKNTDSRCKA RQLELNERTC X₃CDKPRR (Seq. ID No.28), and X₁, X₂, and X₃ are R or a non-basic amino acid, but at least one of X₁, X₂, and X₃ is a non-basic amino acid.

In some useful embodiments of this method of the invention, the VEGF polypeptide sequence variant has the sequence PCSERAKHLF VQDPQTCKCS CKNTDSRCKA RQLELNERTC ACDKPRR (Seq. ID No. 3). In other useful embodiments, the VEGF polypeptide sequence variant has the sequence PCSEAAKHLF VQDPQTCKCS CKNTDSRCKA RQLELNERTC ACDKPRR (Seq. ID No. 4).

In some particularly useful embodiments of this aspect of the invention, the VEGF polypeptide sequence variant has the sequence APMA EGGGQNHHEV VKFMDVYQRS YCHPIETLVD IFQEYPDEIE YIFKPSCVPL MRCGGCCNDE GLECVPTEES NITMQIMRIK PHQGQHIGEM SFLQHNKCEC RPKKDRARQE NPCGPC SERAKHLFVQ DPQTCKCSCK NTDSRCKARQ LELNERTCAC DKPRR (Seq. ID No. 23). In still other useful embodiments, the VEGF polypeptide sequence variant has the sequence APMA EGGGQNHHEV VKFMDVYQRS YCHPIETLVD IFQEYPDEIE YIFKPSCVPL MRCGGCCNDE GLECVPTEES NITMQIMRIK PHQGQHIGEM SFLQHNKCEC RPKKDRARQE NPCGPC SEAAKHLFVQ DPQTCKCSCK NTDSRCKARQ LELNERTCAC DKPRR (Seq. ID No. 24).

In certain embodiments, the disease or condition treated in this aspect of the invention is ischemia associated with coronary artery disease. In particular embodiments, the VEGF polypeptide sequence variant increases collateral vessel formation in ischemic heart disease. In other embodiments, the disease or condition is diabetic neuropathy of the lower extremities. In further embodiments, disease or condition is wound healing. In other embodiments, the disease or condition is cardiovascular disease. In further embodiments, the disease or condition is ischemia.

In certain particularly useful embodiments, the VEGF polypeptide sequence variant causes a lower level of leukostasis than does the corresponding native VEGF polypeptide sequence.

In a further aspect, the invention provides a method of treating a disease or disorder with a VEGF polypeptide having reduced inflammatory side effects by administering a polypeptide that includes a VEGF polypeptide variant with reduced pro-inflammatory activity having one or more alterations of a native VEGF polypeptide sequence that reduces neuropilin-1 receptor binding activity, while substantially maintaining the affinity for VEGR-2 (FLK-1/KDR).

In certain embodiments, the VEGF polypeptide sequence variant has one or more amino acid substitutions of a basic amino acid residue of the native VEGF polypeptide sequence PCSERRKHLF VQDPQTCKCS CKNTDSRCKA RQLELNERTC RCDKPRR (Seq. ID No. 1). In further embodiments, the VEGF polypeptide sequence variant includes the sequence PCSEX₁X₂KHLF VQDPQTCKCS CKNTDSRCKA RQLELNERTC X₃CDKPRR (Seq. ID No.28), and X₁, X₂, and X₃ are R or a non-basic amino acid, but at least one of X₁, X₂, and X₃ is a non-basic amino acid. In certain particularly useful embodiments, the the VEGF polypeptide sequence variant has the sequence PCSERAKHLF VQDPQTCKCS CKNTDSRCKA RQLELNERTC ACDKPRR (Seq. ID No.3). In other useful particularly useful embodiments, the VEGF polypeptide sequence variant has the sequence PCSEAAKHLF VQDPQTCKCS CKNTDSRCKA RQLELNERTC ACDKPRR (Seq. ID No.4).

In certain embodiments of this aspect of the invention, the VEGF polypeptide sequence variant has the sequence: APMA EGGGQNHHEV VKFMDVYQRS YCHPIETLVD IFQEYPDEIE YIFKPSCVPL MRCGGCCNDE GLECVPTEES NITMQIMRIK PHQGQHIGEM SFLQHNKCEC RPKKDRARQE NPCGPC SERAKHLFVQ DPQTCKCSCK NTDSRCKARQ LELNERTCAC DKPRR (Seq. ID No. 23). In other embodiments, VEGF polypeptide sequence variant has the sequence APMA EGGGQNHHEV VKFMDVYQRS YCHPIETLVD IFQEYPDEIE YIFKPSCVPL MRCGGCCNDE GLECVPTEES NITMQIMRIK PHQGQHIGEM SFLQHNKCEC RPKKDRARQE NPCGPC SEAAKHLFVQ DPQTCKCSCK NTDSRCKARQ LELNERTCAC DKPRR (Seq. ID No. 24).

In further embodiments, the VEGF polypeptide sequence variant increases collateral vessel formation in ischemic heart disease. In particular embodiments, the disease or condition treated is diabetic neuropathy of the lower extremities. In certain embodiments, the VEGF polypeptide sequence variant induces less leukostasis than does the corresponding native VEGF polypeptide sequence. In further embodiments, the disease or condition treated is wound healing. In other embodiments, the disease or condition treated is cardiovascular disease. In particular embodiments, the disease or condition is ischemia.

In another aspect, the invention provides a method of identifying an inhibitor of a heparin/VEGF interaction by: detecting a level of heparin/EGF interaction in the presence of a test compound; and comparing the level of heparin/VEGF interaction in the presence of the test compound to the level of heparin/VEGF interaction in the absence of the test compound. In general, the test compound is an inhibitor of the heparin/VEGF interaction if the level of heparin/VEGF interaction in the presence of a test compound is lower than the level of heparin/VEGF interaction in the absence of the test compound.

In certain embodiments, this method further includes the step of identifying a specific inhibitor of a VEGF pro-inflammatory effect that does not interfere with a VEGF pro-angiogenic effect. In general, such specific inhibitors of a VEGF pro-inflammatory effect are identified by detecting a level of VEGF interaction with a VEGF receptor in the presence of the test compound, and comparing the level of VEGF interaction with the VEGF receptor in the presence of the test compound with the level of VEGF interaction with the VEGF receptor in the absence of the test compound. In general, the test compound is a specific inhibitor of a VEGF pro-inflammatory effect if the level of VEGF interaction with the VEGF receptor in the presence of the test compound is substantially the same or greater than the level of VEGF interaction with the VEGF receptor in the absence of the test compound (and the test compound is an inhibitor of a heparin/VEGF interaction, as provided above). In certain embodiments, the VEGF receptor is VEGFR-2 (FLK-1/KDR). In other embodiments, VEGF receptor is VEGFR-1.

In particular embodiments, the test compound is an aptamer. In other embodiments, the test compound is a peptide or a peptidomimetic.

In certain useful embodiments, this method of the invention further provides for co-administering a VEGF polypeptide and a specific inhibitor of a VEGF pro-inflammatory effect that does not interfere with a VEGF pro-angiogenic effect, e.g., as identified above, to a mammalian subject to stimulate angiogenesis with a reduced VEGF pro-inflammatory effect.

In yet another aspect, the invention provides a method of isolating a VEGF polypeptide sequence variant having a reduced affinity for heparin. The method generally includes the steps of: providing a polypeptide that includes a variant of a native VEGF polypeptide sequence, and comparing the level of heparin binding of the polypeptide that includes the variant to the level of heparin binding of the polypeptide comprising the native VEGF polypeptide sequence. In general, the VEGF polypeptide sequence variant is a VEGF polypeptide sequence variant having a reduced affinity for heparin if the level of heparin binding of the polypeptide comprising the variant is lower than the level of heparin binding of the polypeptide comprising the native VEGF polypeptide sequence.

In certain particularly useful embodiments of this aspect of the invention, the VEGF polypeptide sequence variant is a variant of the native VEGF polypeptide sequence PCSERRKHLF VQDPQTCKCS CKNTDSRCKA RQLELNERTC RCDKPRR (Seq. ID No.1). In certain other particularly useful embodiments of this aspect of the invention, the VEGF polypeptide sequence variant is a variant of the native VEGF polypeptide sequence ARQENPCGPC SERRKHLFVQ DPQTCKCSCK NTDSRCKARQ LELNERTCRC DKPRR (Seq. ID No.38; VEGF55).

In particular embodiments, the VEGF polypeptide sequence variant is a substitution of a basic amino acid. In other embodiments, the VEGF polypeptide sequence variant is a deletion of a basic amino acid. In still other useful embodiments, the VEGF polypeptide sequence variant is an insertion adjacent to a basic amino acid.

Aspects of the invention also provide an isolated nucleic acid molecule comprising a sequence that encodes a VEGF variant comprises a modified heparin binding domain; wherein the modified heparin binding domain differs from a native heparin binding domain by comprising mutations such that basic amino acid residues of the native heparin binding domain are substituted with neutral amino acid residues or acidic amino acid residues. The VEGF variant binds heparin at a lower affinity than the native VEGF while maintaining receptor binding function.

The invention, in part, also provides an expression vector for producing a VEGF variant in a host cell. The vector comprises: a) a polynucleotide encoding the VEGF variant; b) transcriptional and translational regulatory sequences functional in the host cell operably linked to the VEGF variant-encoding polynucleotide; and c) a selectable marker.

The invention, in part, also provides a host cell stably transformed and transfected with a polynucleotide encoding a VEGF variant in a manner allowing the expression in the host cell of the VEGF variant.

The invention, in part, also provides a method of visualizing phosphorylation effects triggered by VEGF on p120 or p100. J. M. Staddon et al. (EP1137946B1, the contents of which is incorporated herein by reference in its entirety) provide methods of screening for a substance capable of affecting the serine or threonine phosphorylation state of p120 or p100.

The invention, in part, also provides a method of characterizing the role of the HBD in isoform specific recognition of VEGF165. F. Jucker et al. used NMR spectroscopy ot compare an isolated HBD-Aptamer complex with a full length VEG164-aptamer complex (Lee et al. PNAS, (2005) Vol. 102, 18902-18907, the contents of which is incorporated herein by reference in its entirety).

The invention, in part, further provides a method for designing and screening potentially therapeutic compounds for the treatment of neovascular diseases or diseases related to angiogenesis or inflammation, including but not limited to ocular neovascular disorders, (age-related macular degeneration, diabetic retinopathy and retinopathy of prematurity), psoriasis, rheumatoid arthritis, asthma, inflammatory bowel disease (e.g., Crohn's disease) multiple sclerosis, chronic obstructive pulmonary disease (COPD), allergic rhinitis (hay fever), cardiovascular disease.

The invention, in part, also provides methods for computational modeling of the heparin binding domain of VEGF, such a model being useful in the design of compounds that interact with this domain. The method involves applying mutagenesis data of the VEGF heparin binding domain described herein and the data generated from the x-ray and solution structure, to a computer algorithm capable of generating a three-dimensional model of the heparin binding domain of VEGF and the binding site suitable for use in designing molecules that will act as agonists or antagonists to the polypeptide. The x-ray crystallographic coordinates and solution structure have been disclosed (see Y. A. Muller et al. (1997) Structure 5:1325-1338; C. Wiesmann et al. (1997) Cell 91:695-704; M. A. McTigue et al. (1997) Structure, 7:319-330; and Melissa E. Stauffer et al. (2002) Journal of Biomolecular NMR, 23:57-61). The mutagenesis data showing the functional site of the VEGF heparin binding domain disclosed herein, allow generation of three-dimensional models of the functional site of the VEGF heparin binding domain.

Aspects of the present invention also provide methods for identifying potential modulators of the VEGF heparin binding domain by de novo design of novel drug candidate molecules that bind to and inhibit VEGF heparin binding domain functions or that improve their potency. De novo design comprises of the generation of molecules via the use of computer programs which build and link fragments or atoms into a site based upon steric and electrostatic complementarily, without reference to substrate analog structures. The drug design process begins after the structure of the VEGF heparin binding domain is solved and the region responsible for heparin binding function is determined. An iterative process is then applied to various molecular structures using the computer-generated model to identify potential agonists or antagonists of the heparin binding domain of VEGF. Antagonists and agonists of the VEGF heparin binding domain serve as lead compounds for the design of potentially therapeutic compounds for the treatment of diseases or disorders associated with angiogenesis and inflammation.

In one embodiment, the method for identifying a potential modulator of VEGF heparin binding domain activity, comprising the steps of: a) providing the atomic coordinates of the site responsible for VEGF heparin binding domain function, thereby defining a three-dimensional structure of the site responsible for VEGF heparin binding; b) using the three dimensional structure of the VEGF heparin binding domain to design or select a potential modulator by computer modeling; c) providing the potential modulator; and d) physically contacting the potential modulator with the VEGF heparin binding domain to determine the ability of said potential modulator to modulate VEGF heparin binding domain activity, wherein a modulator of the VEGF heparin binding domain activity is identified. In another embodiment, the potential modulator is designed de novo. In a certain embodiment of the invention, the potential modulator is designed from a known modulator. In another embodiment, the potential modulator is designed from Macugen®.

Aspects of the present invention also provides methods for screening candidate compounds using computational models of the heparin binding domain of VEGF generated using the mutagenesis data of the VEGF heparin binding domain described herein and the data generated from the x-ray and solution structure of VEGF.

The VEGF modulator compounds provided by the invention may be used as anti-inflammatory, anti-vascular permeability, immunosuppressant, anti-hypertension agents.

The present invention, in part, also provides methods for screening VEGF variants using in vitro or in vivo assays.

The present invention, in part, also provides methods for screening VEGF antagonists using in vitro or in vivo assays.

In one embodiment, the invention provides a method for assessing a candidate compound for its ability to inhibit the function of the heparin binding domain of VEGF wherein the function of the receptor binding domain of VEGF is maintained. The method comprises: (a) assaying the candidate compound for its ability to inhibit heparin binding; (b) assaying the candidate compound for its ability to inhibit receptor binding; and (c) determining the ability of the candidate compound to inhibit heparin binding while maintaining receptor binding function. Any suitable assay known in the art may be used. Suitable assays include, but are not limited to those shown below in Examples 2-5.

In another aspect, the invention provides methods of inhibiting VEGF164 induced leukostasis. In one embodiment, the method of inhibiting VEGF164 induced leukostasis comprises administering a soluble heparin binding domain. In one particular embodiment, the soluble heparin binding domain comprises a polypeptide having the sequence of VEGF55.

The patent and scientific literature referred to herein establishes knowledge that is available to those of skill in the art. The issued U.S. patents, allowed applications, published foreign applications, and references, including GenBank database sequences, that are cited herein are hereby incorporated by reference to the same extent as if each was specifically and individually indicated to be incorporated by reference in their entirety.

Definitions

As used herein, the following terms and phrases shall have the meanings set forth below. Unless defined otherwise, all technical and scientific terms used herein have the same meaning as commonly understood to one of ordinary skill in the art to which this invention belongs.

As used herein, the term “alteration,” such as in the phrase “one or more alterations of a native VEGF polypeptide sequence” refers to amino acid substitutions, amino acid deletions and amino acid insertions, but not protein truncations (e.g. C-terminal protein truncations such as effected by insertion of a stop codon or proteolytic removal of a C-terminal portion of the protein).

By “antagonist” is meant an agent that inhibits, either partially or fully, the activity or production of a target molecule. In particular, the term “antagonist,” as applied selectively herein, means an agent capable of decreasing levels of VEGF or VEGFR protein levels or protein activity. Exemplary forms of antagonists include, for example, proteins, polypeptides, peptides (such as cyclic peptides), antibodies or antibody fragments, peptide mimetics, nucleic acid molecules, antisense molecules, ribozymes, aptamers, RNAi molecules, and small organic molecules. Exemplary non-limiting mechanisms of antagonist inhibition of the VEGF/VEGFR ligand/receptor targets include repression of ligand synthesis and/or stability (e.g., using, antisense, ribozymes or RNAi compositions targeting the ligand gene/nucleic acid), blocking of binding of the ligand to its cognate receptor (e.g., using anti-ligand aptamers, antibodies or a soluble, decoy cognate receptor), repression of receptor synthesis and/or stability (e.g., using, antisense, ribozymes or RNAi compositions targeting the ligand receptor gene/nucleic acid), blocking of the binding of the receptor to its cognate receptor (e.g., using receptor antibodies) and blocking of the activation of the receptor by its cognate ligand (e.g., using receptor tyrosine kinase inhibitors). In addition, the antagonist may directly or indirectly inhibit the target molecule.

The term “antibody” as used herein is intended to include whole antibodies, e.g., of any isotype (IgG, IgA, IgM, IgE, etc.), and includes fragments thereof which recognize and are also specifically reactive with vertebrate (e.g., mammalian) protein, carbohydrates, etc. Antibodies can be fragmented using conventional techniques and the fragments screened for utility in the same manner as described above for whole antibodies. Thus, the term includes segments of proteolytically cleaved or recombinantly-prepared portions of an antibody molecule that are capable of selectively reacting with a certain protein. Non-limiting examples of such proteolytic and/or recombinant fragments include Fab, F (ab′)2, Fab′, Fv, and single chain antibodies (scFv) containing a V[L] and/or V[H] domain joined by a peptide linker. The scFv's may be covalently or noncovalently linked to form antibodies having two or more binding sites. The subject invention includes polyclonal, monoclonal, or other purified preparations of antibodies and recombinant antibodies.

The term “aptamer,” used herein interchangeably with the term “nucleic acid ligand,” means a nucleic acid that, through its ability to adopt a specific three dimensional conformation, binds to and has an antagonizing (i.e., inhibitory) effect on a target. The target of the present invention is VEGF (or one of its cognate receptors VEGFR), and hence the term VEGF aptamer or nucleic acid ligand (or VEGFR aptamer or nucleic acid ligand) is used. Inhibition of the target by the aptamer may occur by binding of the target, by catalytically altering the target, by reacting with the target in a way which modifies/alters the target or the functional activity of the target, by covalently attaching to the target as in a suicide inhibitor, by facilitating the reaction between the target and another molecule. Aptamers may be comprised of multiple ribonucleotide units, deoxyribonucleotide units, or a mixture of both types of nucleotide residues. Aptamers may further comprise one or more modified bases, sugars or phosphate backbone units as described in further detail herein.

By “antibody antagonist” is meant an antibody molecule as herein defined which is able to block or significantly reduce one or more activities of a target VEGF. For example, a VEGF inhibitory antibody may inhibit or reduce the ability of VEGF to stimulate angiogenesis.

A nucleotide sequence is “complementary” to another nucleotide sequence if each of the bases of the two sequences matches, i.e., are capable of forming Watson Crick base pairs. The term “complementary strand” is used herein interchangeably with the term “complement.” The complement of a nucleic acid strand can be the complement of a coding strand or the complement of a non-coding strand.

The phrases “conserved residue” or “conservative amino acid substitution” refer to grouping of amino acids on the basis of certain common properties. A functional way to define common properties between individual amino acids is to analyze the normalized frequencies of amino acid changes between corresponding proteins of homologous organisms. According to such analyses, groups of amino acids may be defined where amino acids within a group exchange preferentially with each other, and therefore resemble each other most in their impact on the overall protein structure (Schulz, G. E. and R. H. Schirmer, Principles of Protein Structure, Springer-Verlag). Examples of amino acid groups defined in this manner include:
(i) a charged group, consisting of Glu and Asp, Lys, Arg and His,
(ii) a positively-charged group, (i.e., basic amino acid) consisting of Lys, Arg and His (i.e., K, R and H),
(iii) a negatively-charged group, (i.e., acidic amino acid) consisting of Glu and Asp (i.e., E and D),
(iv) an aromatic group, consisting of Phe, Tyr and Trp,
(v) a nitrogen ring group, consisting of His and Trp,
(vi) a large aliphatic nonpolar group, consisting of Val, Leu and Ile,
(vii) a slightly-polar group, consisting of Met and Cys,
(viii) a small-residue group, consisting of Ser, Thr, Asp, Asn, Gly, Ala, Glu, Gln and Pro,
(ix) an aliphatic group consisting of Val, Leu, Ile, Met and Cys, and
(x) a small hydroxyl group consisting of Ser and Thr.

In addition to the groups presented above, each amino acid residue may form its own group, and the group formed by an individual amino acid may be referred to simply by the one and/or three letter abbreviation for that amino acid commonly used in the art.

The term “interact” as used herein is meant to include detectable relationships or association (e.g., biochemical interactions) between molecules, such as interaction between protein-protein, protein-nucleic acid, nucleic acid-nucleic acid, and protein-small molecule or nucleic acid-small molecule in nature.

The term “interacting protein” refers to protein capable of interacting, binding, and/or otherwise associating to a protein of interest, such as for example a VEGF protein, or their corresponding cognate receptors.

As used herein, a peptide is said to be “isolated” or “purified” when it is substantially free of homologous cellular material or chemical precursors or other chemicals. The peptides of the present invention can be purified to homogeneity or other degrees of purity. The level of purification will be based on the intended use.

The term “isolated” as used herein with respect to nucleic acids, such as DNA or RNA, refers to molecules separated from other DNAs, or RNAs, respectively that are present in the natural source of the macromolecule. Similarly the term “isolated” as used herein with respect to polypeptides refers to protein molecules separated from other proteins that are present in the source of the polypeptide. The term isolated as used herein also refers to a nucleic acid or peptide that is substantially free of cellular material, viral material, or culture medium when produced by recombinant DNA techniques, or chemical precursors or other chemicals when chemically synthesized.

“Isolated nucleic acid” is meant to include nucleic acid fragments, which are not naturally occurring as fragments and would not be found in the natural state. The term “isolated” is also used herein to refer to polypeptides, which are isolated from other cellular proteins and is meant to encompass both purified and recombinant polypeptides.

As used herein, the term “substantially free of cellular material” includes preparations of the peptide having less than about 30% (by dry weight) other proteins (i.e., contaminating protein), less than about 20% other proteins, less than about 10% other proteins, or less than about 5% other proteins. When the peptide is recombinantly produced, it can also be substantially free of culture medium, i.e., culture medium represents less than about 20% of the volume of the protein preparation.

As used herein, the term “substantially free of chemical precursors or other chemicals” includes preparations of the peptide in which it is separated from chemical precursors or other chemicals that are involved in its synthesis. In certain embodiments, the language “substantially free of chemical precursors or other chemicals” includes preparations of the VEGF peptide having less than about 30% (by dry weight) chemical precursors or other chemicals, but the invention also includes embodiments with less than about 20% chemical precursors or other chemicals, less than about 10% chemical precursors or other chemicals, or less than about 5% chemical precursors or other chemicals.

The “level of expression of a gene in a cell” refers to the level of mRNA, as well as pre-mRNA nascent transcript(s), transcript processing intermediates, mature mRNA(s) and degradation products, encoded by the gene in the cell, as well as the level of protein translated from that gene.

As used herein, the term “nucleic acid” refers to polynucleotides such as deoxyribonucleic acid (DNA), and, where appropriate, ribonucleic acid (RNA). The term should also be understood to include, as equivalents, analogs of either RNA or DNA made from nucleotide analogs, and, as applicable to the embodiment being described, single (sense or antisense) and double-stranded polynucleotides, ESTs, chromosomes, cDNAs, mRNAs, siRNAs and rRNAs are representative examples of molecules that may be referred to as nucleic acids.

The term “oligonucleotide” refers to an oligomer or polymer of nucleotide or nucleoside monomers consisting of naturally occurring bases, sugars and inter-sugar (backbone) linkages. The term also includes modified or substituted oligomers comprising non-naturally occurring monomers or portions thereof, which function similarly. Incorporation of substituted oligomers is based on factors including enhanced cellular uptake, or increased nuclease resistance and chosen as is known in the art. The entire oligonucleotide or only portions thereof may contain the substituted oligomers.

Oligonucleotides are chemically synthesized by known methods (such as phosphotriester, phosphite, or phosphoramidite chemistry, using solid phase techniques such as described in EP Patent Publication No. 266,032 published May 4, 1988, or via deoxynucleoside H-phosphonate intermediates as described by Froehler et al. (1986), Nucl. Acids Res. 14:5399-5407). They may be then purified on polyacrylamide gels.

The term “percent identical” refers to sequence identity between two amino acid sequences or between two nucleotide sequences. Identity can each be determined by comparing a position in each sequence, which may be aligned for purposes of comparison. When an equivalent position in the compared sequences is occupied by the same base or amino acid, then the molecules are identical at that position; when the equivalent site occupied by the same or a similar amino acid residue (e.g., similar in steric and/or electronic nature), then the molecules can be referred to as homologous (similar) at that position. Expression as a percentage of homology, similarity, or identity refers to a function of the number of identical or similar amino acids at positions shared by the compared sequences. Various alignment algorithms and/or programs may be used, including Hidden Markov Model (HMM), FASTA and BLAST. HNiM, FASTA and BLAST are available through the National Center for Biotechnology Information, National Library of Medicine, National Institutes of Health, Bethesda, Md. and the European Bioinformatic Institute EBI. In one embodiment, the percent identity of two sequences that can be determined by these GCG programs with a gap weight of 1, e.g., each amino acid gap is weighted as if it were a single amino acid or nucleotide mismatch between the two sequences. Other techniques for alignment are described in Methods in Enzymology, vol. 266: Computer Methods for Macromolecular Sequence Analysis (1996), ed. Doolittle, Academic Press, Inc., a division of Harcourt Brace & Co., San Diego, Calif., USA. Where desirable, an alignment program that permits gaps in the sequence is utilized to align the sequences. The Smith Waterman is one type of algorithm that permits gaps in sequence alignments (see (1997) Meth. Mol. Biol. 70: 173-187). Also, the GAP program using the Needleman and Wunsch alignment method can be utilized to align sequences. More techniques and algorithms including use of the HMM are described in Sequence, Structure, and Databanks: A Practical Approach (2000), ed. Oxford University Press, Incorporated and in Bioinformatics: Databases and Systems (1999) ed. Kluwer Academic Publishers. An alternative search strategy uses MPSRCH software, which runs on a MASPAR computer. MPSRCH uses a Smith-Watermnan algorithm to score sequences on a massively parallel computer. This approach improves ability to pick up distantly related matches, and is especially tolerant of small gaps and nucleotide sequence errors. Nucleic acid-encoded amino acid sequences can be used to search both protein and DNA databases. Databases with individual sequences are described in Methods in Enzymology, ed. Doolittle, supra. Databases include Genbank, EMBL, and DNA Database of Japan (DDBJ).

The “profile” of an aberrant, e.g., tumor cell's biological state refers to the levels of various constituents of a cell that change in response to the disease state. Constituents of a cell include levels of RNA, levels of protein abundances, or protein activity levels.

The term “protein” is used interchangeably herein with the terms “peptide” and “polypeptide”. The term “recombinant protein” refers to a protein of the present invention which is produced by recombinant DNA techniques, wherein generally DNA encoding the expressed protein or RNA is inserted into a suitable expression vector which is in turn used to transform a host cell to produce the heterologous protein or RNA. Moreover, the phrase “derived from,” with respect to a recombinant gene encoding the recombinant protein is meant to include within the meaning of “recombinant protein” those proteins having an amino acid sequence of a native protein, or an amino acid sequence similar thereto which is generated by mutations, including substitutions and deletions, of a naturally occurring protein.

As used herein, the term “transgene” means a nucleic acid sequence (encoding, e.g., one of the target nucleic acids, or an antisense transcript thereto), which has been introduced into a cell. A transgene could be partly or entirely heterologous, i.e., foreign, to the transgenic animal or cell into which it is introduced, or, is homologous to an endogenous gene of the transgenic animal or cell into which it is introduced, but which is designed to be inserted, or is inserted, into the animal's genome in such a way as to alter the genome of the cell into which it is inserted (e.g., it is inserted at a location which differs from that of the natural gene or its insertion results in a knockout). A transgene can also be present in a cell in the form of an episome. A transgene can include one or more transcriptional regulatory sequences and any other nucleic acid, such as introns, that may be necessary for optimal expression of a selected nucleic acid.

By “neovascular disorder” is meant a disorder characterized by altered or unregulated angiogenesis other than one accompanying oncogenic or neoplastic transformation, i.e., cancer. Examples of neovascular disorders include psoriasis, rheumatoid arthritis, and ocular neovascular disorders including diabetic retinopathy and age-related macular degeneration.

As used herein, the terms “neovascularization” and “angiogenesis” are used interchangeably. Neovascularization and angiogenesis refer to the generation of new blood vessels into cells, tissue, or organs. The control of angiogenesis is typically altered in certain disease states and, in many cases; the pathological damage associated with the disease is related to altered, unregulated, or uncontrolled angiogenesis. Persistent, unregulated angiogenesis occurs in a multiplicity of disease states, including those characterized by the abnormal growth by endothelial cells, and supports the pathological damage seen in these conditions including leakage and permeability of blood vessels.

By “ocular neovascular disorder” is meant a disorder characterized by altered or unregulated angiogenesis in the eye of a patient. Exemplary, nonlimiting ocular neovascular disorders include optic disc neovascularization, iris neovascularization, retinal neovascularization, choroidal neovascularization, corneal neovascularization, vitreal neovascularization, glaucoma, pannus, pterygium, macular edema, diabetic retinopathy, diabetic macular edema, vascular retinopathy, retinal degeneration, uveitis, inflammatory diseases of the retina, and proliferative vitreoretinopathy.

By “inflammatory disorder” is meant a disorder characterized by altered or unregulated leukocyte recruitment. Examples of inflammatory disorders include but are not limited to rheumatoid arthritis, asthma, inflammatory bowel disease (e.g., Crohn's disease) multiple sclerosis, chronic obstructive pulmonary disease (COPD), allergic rhinitis (hay fever), cardiovascular disease.

By “neuron disorder” is meant a disorder characterized by a physiological dysfunction or death of neurons. Neurons may be present in the central nervous system or peripheral nervous system. A non-limited list of such disorders comprises neurodegenerative disorders, Alzheimer's disease, Parkinson's disease, Huntington's disease, prion diseases, amyotrophic lateral sclerosis (ALS, Lou Gherig' disease), Shy-Drager Syndrome, Progressive Supranuclear Palsy, Lewy Body Disease, neuronopathies and motor neuron disorders and other degenerative neuron disorders.

Other neuron disorders include, but are not limited to, dementia, frontotemporal lobe dementia, ischemic disorders (e.g. cerebral or spinal cord infarction and ischemia, chronic ischernic brain disease, and stroke), kaumas (e.g. caused by physical injury or surgery, and compression injuries), affective disorders (e.g. stress, depression and post-traumatic depression), neuropsychiatric disorders (e. g. schizophrenia, multiple sclerosis, and epilepsy); learning and memory disorders; and ocular neuron disorders. Neuron disorders also include those characterized by the death of neurons induced by, for example, semaphorin 3A.

“Ocular neuron disorder” include, but are not limited to, retina or optic nerve optic nerve disorders, optic nerve damage and optic neuropathies, disorders of the optic nerve or visual pathways, toxic amblyopia, optic atrophy, higher visual pathway lesions, disorders of ocular motility, third cranial nerve palsies, fourth cranial nerve palsies, sixth cranial nerve palsies, internuclear ophthalmoplegia, gaze palsies, and free radical induced eye disorders.

Optic neuropathies include, but are not limited to, ischemic optic neuropathy, inflammation of the optic nerve, bacterial infection of the optic nerve, optic neuritis, optic neuropathy, and papilledema (choked disk), papillitis (optic neuritis), retrobulbar neuritis, optic neuritis (ON), anterior ischaemic optic neuropathy (AION), commotio retinae, glaucoma, macular degeneration, retinitis pigmentosa, retinal detachment, retinal tears or holes, diabetic retinopathy and iatrogenic retinopathy.

One particular ocular neuron disorder is glaucoma. Types of glaucoma include, but are not limited to, primary glaucoma, chronic open-angle glaucoma, acute or chronic angle-closure, congenital (infantile) glaucoma, secondary glaucoma, and absolute glaucoma.

The term “treating” a neovascular disease in a subject or “treating” a subject having a neovascular disease refers to subjecting the subject to a pharmaceutical procedure, e.g., the administration of a drug, such that at least one symptom of the neovascular disease is decreased. Accordingly, the term “treating” as used herein is intended to encompass curing as well as ameliorating at least one symptom of the neovascular condition or disease. Thus, “treating” as used herein, includes administering or prescribing a pharmaceutical composition for the treatment or prevention of an ocular neovascular disorder.

By “patient” is meant any animal. The term “animal” includes mammals, including, but is not limited to, humans and other primates. The term also includes domesticated animals, such as cows, hogs, sheep, horses, dogs, and cats.

By “VEGF,” or “vascular endothelial growth factor,” is meant a mammalian vascular endothelial growth factor that affects angiogenesis or an angiogenic process. As used herein, the term “VEGF” includes the various subtypes of VEGF (also known as vascular permeability factor (VPF) and VEGF-A) (arising by, e.g., alternative splicing of the VEGF-A/VPF gene including VEGF121, VEGF165 and VEGF189. Further, as used herein, the term “VEGF” refers to VEGF-related angiogenic factors such as PIGF (placenta growth factor), VEGF-B, VEGF-C, VEGF-D and VEGF-E that act through a cognate VEFG receptor to stimulate angiogenesis or an angiogenic process. In particular, the term “VEGF” means any member of the class of growth factors that (i) bind to a VEGF receptor such as VEGFR-1 (Flt-1), VEGFR-2 (KDR/Flk-1), or VEGFR-3 (FLT-4); (ii) activates a tyrosine kinase activity associated with the VEGF receptor; and (iii) thereby affects angiogenesis or an angiogenic process. The term “VEGF” is meant to include both a “VEGF” polypeptide and its corresponding “VEGF” encoding gene or nucleic acid.

By “VEGF modulator” is meant an agent that reduces, inhibits, increases or activates either partially or fully, the activity or production of a VEGF. A VEGF modulator may be a VEGF antagonist or VEGF agonist.

By “VEGF antagonist” is meant an agent that reduces, or inhibits, either partially or fully, the activity or production of a VEGF. A VEGF antagonist may directly or indirectly reduce or inhibit a specific VEGF such as VEGF165. A VEGF antagonist may directly or indirectly inhibit a specific function of a VEGF. For example, a VEGF antagonist may inhibit the function of the heparin binding domain while not inhibiting the function of the receptor binding domain. Furthermore, “VEGF antagonists” consistent with the above definition of “antagonist,” may include agents that act on either a VEGF ligand or its cognate receptor so as to reduce or inhibit a VEGF-associated receptor signal. Examples of such “VEGF antagonists” thus include, for example: antisense, ribozymes or RNAi compositions targeting a VEGF nucleic acid; anti-VEGF aptamers, anti- VEGF antibodies or soluble VEGF receptor decoys that prevent binding of a VEGF to its cognate receptor; antisense, ribozymes, or RNAi compositions targeting a cognate VEGF receptor (VEGFR) nucleic acid; anti-VEGFR aptamers or anti-VEGFR antibodies that bind to a cognate VEGFR receptor; and VEGFR tyrosine kinase inhibitors.

By “VEGF agonist” is meant an agent that increases or activates either partially or fully, the activity or production of a VEGF.

By “an amount sufficient to suppress a neovascular disorder” is meant the effective amount of an antagonist required to treat or prevent a neovascular disorder or symptom thereof. The “effective amount” of active antagonists used to practice the present invention for therapeutic treatment of conditions caused by or contributing to the neovascular disorder varies depending upon the manner of administration, anatomical location of the neovascular disorder, the age, body weight, and general health of the patient. Ultimately, a physician or veterinarian will decide the appropriate amount and dosage regimen. Such amount is referred to as an amount sufficient to suppress a neovascular disorder (see, e.g., Remington: The Science and Practice of Pharmacy, (20th ed.) ed. A. R. Gennaro, 2000, Lippincott Williams & Wilkins, Philadelphia, Pa.).

Other features and advantages of the invention will be. apparent from the following detailed description, and from the claims.

A “variant” of polypeptide X refers to a polypeptide having the amino acid sequence of peptide X in which is altered in one or more amino acid residues. A variant may have “conservative” changes, wherein a substituted amino acid has similar structural or chemical properties (e.g., replacement of leucine with isoleucine). A variant may have “nonconservative” changes (e.g., replacement of arginine with alanine). A variant may have secondary or tertiary structure altering changes. A variant may have non-secondary structure altering or non-tertiary structure altering changes. Variations may also include amino acid deletions or insertions, or both. Guidance in determining which amino acid residues may be substituted, inserted, or deleted without abolishing biological or immunological activity may be found using computer programs well known in the art, for example, LASERGENE software (DNASTAR).

The term “variant,” when used in the context of a polynucleotide sequence, may encompass a polynucleotide sequence related to that of gene or the coding sequence thereof. This definition may also include, for example, “allelic,” “splice,” “species,” or “polymorphic” variants. A splice variant may have significant identity to a reference molecule, but generally has a greater or lesser number of polynucleotides due to alternative splicing of exons during mRNA processing. The corresponding polypeptide may possess additional functional domains or an absence of domains. Species variants are polynucleotide sequences that vary from one species to another. The resulting polypeptides generally have significant amino acid identity relative to each other. A polymorphic variant is a variation in the polynucleotide sequence of a particular gene between individuals of a given species.

The term “VEGF variant” refers to VEGF molecules that contain a modification(s) in the heparin binding domain that results in a modification of the function of the heparin binding domain or that has a lower affinity to heparin compared with native material. Such modifications may affect the conformational structure of the resultant variant, hence the use of the term “structural alteration” in respect of such “modifications”. These modifications may be the result of DNA mutagenesis so as to create molecules having different amino acids from those found in the native material. In particular, as the heparin binding domain contains a relatively large number of positively charged amino acids, the binding of that domain with heparin could be based upon ionic interactions. Accordingly, certain embodiments replace positively charged amino acids with negatively or neutrally charged amino acids. Thus, aspects of the present invention as directed to any modification to the heparin binding domain of VEGF that results in a molecule that has modified heparin binding domain function or has a lower affinity to heparin.

The term “vector” refers to a nucleic acid molecule capable of transporting another nucleic acid to which it has been linked. One type of useful vector is an episome, i.e., a nucleic acid capable of extra-chromosomal replication. Useful vectors are those capable of autonomous replication and/or expression of nucleic acids to which they are linked. Vectors capable of directing the expression of genes to which they are operatively linked are referred to herein as “expression vectors”. In general, expression vectors of utility in recombinant DNA techniques are often in the form of “plasmids” which refer generally to circular double stranded DNA loops which, in their vector form are not bound to the chromosome. In the present specification, “plasmid” and “vector” are used interchangeably as the plasmid is the most commonly used form of vector. However, the invention is intended to include such other forms of expression vectors which serve equivalent functions and which become known in the art subsequently hereto.

“Transfection” refers to the taking up of nucleic acid, e.g., an expression vector, by a host cell whether or not any coding sequences are in fact expressed. Numerous methods of transfection are known to the ordinarily skilled artisan, for example, CaPO₄ and electroporation. Successful transfection is generally recognized when any indication of the operation of this vector occurs within the host cell.

“Transformation” means introducing DNA into an organism so that the DNA is replicable, either as an extrachromosomal element or by chromosomal integrant. Depending on the host cell used, transformation is done using standard techniques appropriate to such cells. The calcium treatment employing calcium chloride, as described by Cohen, S. N. (1972) Proc. Natl. Acad. Sci. (USA), 69:2110 and Mandel et al. (1970) J. Mol. Biol. 53:154, is generally used for prokaryotes or other cells that contain substantial cell-wall barriers. For mammalian cells without such cell walls, the calcium phosphate precipitation method of Graham, F. and van der Eb, A., (1978) Virology, 52:456-457 is particularly useful. General aspects of mammalian cell host system transformations have been described by Axel in U.S. Pat. No. 4,399,216 issued Aug. 16, 1983. Transformations into yeast are typically carried out according to the method of Van Solingen, P., et al., (1977) J. Bact., 130:946 and Hsiao, C. L., et al. (1979) Proc. Natl. Acad. Sci. (USA) 76:3829. However, other methods for introducing DNA into cells such as by nuclear injection or by protoplast fusion may also be used. “Site-directed mutagenesis” is a technique standard in the art, and is conducted using a synthetic oligonucleotide primer complementary to a single-stranded phage DNA to be mutagenized except for limited mismatching, representing the desired mutation. Briefly, the synthetic oligonucleotide is used as a primer to direct synthesis of a strand complementary to the phage, and the resulting double-stranded DNA is transformed into a phage-supporting host bacterium. Cultures of the transformed bacteria are plated in top agar, permitting plaque formation from single cells that harbor the phage. Theoretically, 50% of the new plaques contain the phage having, as a single strand, the mutated form; 50% have the original sequence. The plaques are hybridized with kinased synthetic primer at a temperature that permits hybridization of an exact match, but at which the mismatches with the original strand are sufficient to prevent hybridization. Plaques that hybridize with the probe are then selected and cultured, and the DNA is recovered.

“Operably linked” refers to juxtaposition such that the normal function of the components can be performed. Thus, a coding sequence “operably linked” to control sequences refers to a configuration wherein the coding sequence can be expressed under the control of these sequences and wherein the DNA sequences being linked are contiguous and, in the case of a secretory leader, contiguous and in reading phase. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Linking is accomplished by ligation at convenient restriction sites. If such sites do not exist, then synthetic oligonucleotide adaptors or linkers are used in accord with conventional practice.

“Control sequences” refer to DNA sequences necessary for the expression of an operably linked coding sequence in a particular host organism. Control sequences that are suitable for prokaryotes, for example, include a promoter, optionally an operator sequence, a ribosome binding site, and possibly, other as yet poorly understood sequences. Eukaryotic cells are known to utilize promoters, polyadenylation signals, and enhancers.

“Expression system” refers to DNA sequences containing a desired coding sequence and control sequences in operable linkage, so that hosts transformed with these sequences are capable of producing the encoded proteins. To effect transformation, the expression system may be included on a vector; however, the relevant DNA may then also be integrated into the host chromosome.

As used herein, “cell,” “cell line,” and “cell culture” are used interchangeably and all such designations include progeny. Thus, “transformants” or “transformed cells” include the primary subject cell and cultures derived therefrom without regard for the number of transfers. It is also understood that all progeny may not be precisely identical in DNA content, due to deliberate or inadvertent mutations. Mutant progeny that have the same functionality as screened for in the originally transformed cell are included. Where distinct designations are intended, it will be clear from the context.

“Plasmids” are designated by a lower case “p” preceded and/or followed by capital letters and/or numbers. The starting plasmids herein are commercially available, are publicly available on an unrestricted basis, or can be constructed from such available plasmids in accord with published procedures. In addition, other equivalent plasmids are known in the art and will be apparent to the ordinary artisan.

“Digestion” of DNA refers to catalytic cleavage of the DNA with an enzyme that acts only at certain locations in the DNA. Such enzymes are called restriction enzymes, and the sites for which each is specific is called a restriction site. The various restriction enzymes used herein are commercially available and their reaction conditions, cofactors, and other requirements as established by the enzyme suppliers are used. Restriction enzymes commonly are designated by abbreviations composed of a capital letter followed by other letters representing the microorganism from which each restriction enzyme originally was obtained and then a number designating the particular enzyme. In general, about 1 mg of plasmid or DNA fragment is used with about 1-2 units of enzyme in about 20 ml of buffer solution. Appropriate buffers and substrate amounts for particular restriction enzymes are specified by the manufacturer. Incubation of about 1 hour at about 37° C. is ordinarily used, but may vary in accordance with the supplier's instructions. After incubation, protein is removed by extraction with phenol and chloroform, and the digested nucleic acid is recovered from the aqueous fraction by precipitation with ethanol. Digestion with a restriction enzyme infrequently is followed with bacterial alkaline phosphatase hydrolysis of the terminal 5′ phosphates to prevent the two restriction cleaved ends of a DNA fragment from “circularizing” or forming a closed loop that would impede insertion of another DNA fragment at the restriction site. Unless otherwise stated, digestion of plasmids is not followed by 5′ terminal dephosphorylation. Procedures and reagents for dephosphorylation are conventional (T. Maniatis et al. 1982, Molecular Cloning: A Laboratory Manual (New York: Cold Spring Harbor Laboratory, 1982) pp. 133-134).

“Recovery” or “isolation” of a given fragment of DNA from a restriction digest means separation of the digest on polyacrylamide or agarose gel by electrophoresis, identification of the fragment of interest by comparison of its mobility versus that of marker DNA fragments of known molecular weight, removal of the gel section containing the desired fragment, and separation of the gel from DNA. This procedure is known generally in the art. For example, see R. Lawn et al., (1981) Nucleic Acids Res. 9:6103-6114, and D. Goeddel et al., (1980) Nucleic Acids Res. 8:4057.

“Southern Analysis” is a method by which the presence of DNA sequences in a digest or DNA-containing composition is confirmed by hybridization to a known, labeled oligonucleotide or DNA fragment. For the purposes herein, unless otherwise provided, Southern analysis shall mean separation of digests on 1 percent agarose, denaturation, and transfer to nitrocellulose by the method of E. Southern, (1975) J. Mol. Biol. 98:503-517, and hybridization as described by T. Maniatis et al., (1978) Cell 15:687-701.

“Ligation” refers to the process of forming phosphodiester bonds between two double stranded nucleic acid fragments (T. Maniatis et al. 1982, Molecular Cloning: A Laboratory Manual (New York: Cold Spring Harbor Laboratory, 1982) pp. 133-134). Unless otherwise provided, ligation may be accomplished using known buffers and conditions with 10 units of T4 DNA ligase (“ligase”) per 0.5 mg of approximately equimolar amounts of the DNA fragments to be ligated.

“Preparation” of DNA from transformants means isolating plasmid DNA from microbial culture. Unless otherwise provided, the alkaline/SDS method of Maniatis et al. 1982, supra, p. 90, may be used.

VEGF proteins are important stimuli for the growth of new blood vessels throughout the body, especially in the eye. Therapy directed at inhibiting VEGF biological activities provides a method for treating or preventing the neovascular disorder. Accordingly, the invention features VEGF modulator compositions and methods and compositions for suppressing a neovascular disorder.

The present VEGF modulator compositions and methods and according to the invention are especially useful for treating any number of ophthamalogical diseases and disorders marked by the development of ocular neovascularization. Such diseases and disorders include, but are not limited to, optic disc neovascularization, iris neovascularization, retinal neovascularization, choroidal neovascularization, corneal neovascularization, vitreal neovascularization, glaucoma, pannus, pterygium, macular edema, diabetic macular edema, vascular retinopathy, retinal degeneration, macular degeneration, uveitis, inflammatory diseases of the retina, and proliferative vitreoretinopathy.

Therapies according to the invention may be performed alone or in conjunction with another therapy and may be provided at home, a doctor's office, a clinic, a hospital's outpatient department, or a hospital. Treatment generally begins at a hospital so that the doctor can observe the therapy's effects closely and make any adjustments that are needed. The duration of the therapy depends on the type of neovascular disorder being treated, the age and condition of the patient, the stage and type of the patient's disease, and how the patient responds to the treatment. Additionally, a person having a greater risk of developing a neovascular disorder (e.g., a diabetic patient) may receive treatment to inhibit or delay the onset of symptoms.

The present invention has several advantages. The VEGF variants of the present invention promote angiogenesis without the promoting inflammation. The VEGF antagonists of the present invention prevent or decrease leukostasis without preventing or decreasing angiogenesis. A significant advantage of the compounds and methods provided by the present invention is their specificity for the treatment of a neovascular disorder. Such specificity allows for the administration of low doses and provides less toxicity and side effects.

For use in combination therapy, the dosage and frequency of administration of each component of the combination can be controlled independently. For example, one component may be administered three times per day, while the second component may be administered once per day. Combination therapy may be given in on-and-off cycles that include rest periods so that the patient's body has a chance to recover from any as yet unforeseen side-effects. The components may also be formulated together such that one administration delivers both components.

VEGF is a secreted disulfide-linked homodimer that selectively stimulates endothelial cells to proliferate, migrate, and produce matrix-degrading enzymes (Conn et al., (1990) Proc. Natl. Acad. Sci. (USA) 87:1323-1327); Ferrara and Henzel (1989) Biochem. Biophys. Res. Commun.161: 851-858); Pepper et al., (1991) Biochem. Biophys. Res. Commun. 181:902-906; Unemori et al., (1992) J. Cell. Physiol. 153:557-562), all of which are processes required for the formation of new vessels. VEGF occurs in four forms (VEGF-121, VEGF-165, VEGF-189, VEGF-206) as a result of alternative splicing of the VEGF gene (Houck et al., (1991) Mol. Endocrinol. 5:1806-1814; Tischer et al., (1991) J. Biol. Chem. 266:11947-11954). The two smaller forms are diffusible whereas the larger two forms remain predominantly localized to the cell membrane as a consequence of their high affinity for heparin. VEGF-165 also binds to heparin and is the most abundant form. VEGF-121, the only form that does not bind to heparin, appears to have a lower affinity for VEGF receptors (Gitay-Goren et al., (1996) J. Biol. Chem. 271:5519-5523) as well as lower mitogenic potency (Keyt et al., (1996) J. Biol. Chem. 271:7788-7795). The biological effects of VEGF are mediated by two tyrosine kinase receptors (Flt-1 and Flk-1/KDR) whose expression is highly restricted to cells of endothelial origin (de Vries et al., (1992) Science 255:989-991; Millauer et al., (1993) Cell 72:835-846; Terman et al., (1991) Oncogene 6:519-524). While the expression of both functional receptors is required for high affinity binding, the chemotactic and mitogenic signaling in endothelial cells appears to occur primarily through the KDR receptor (Park et al., (1994) J. Biol. Chem. 269:25646-25654; Seetharam et al., (1995) Oncogene 10:135-147; Waltenberger et al., (1994) J. Biol.Chem. 26988-26995). The importance of VEGF and VEGF receptors for the development of blood vessels has recently been demonstrated in mice lacking a single allele for the VEGF gene (Carrneliet et al., (1996) Nature 380:435-439; Ferrara et al., (1996) Nature 380:439-442) or both alleles of the Flt-1 (Fong et al., (1995) Nature 376:66-70) or Flk-1 genes (Shalaby et al., (1995) Nature 376:62-66). In each case, distinct abnormalities in vessel formation were observed resulting in embryonic lethality.

Compensatory angiogenesis induced by tissue hypoxia is now known to be mediated by VEGF (Levy et al., (1996) J. Biol. Chem. 2746-2753); Shweiki et al., (1992) Nature 359:843-845). Studies in humans have shown that high concentrations of VEGF are present in the vitreous in angiogenic retinal disorders but not in inactive or non-neovascularization disease states. Human choroidal tissue excised after experimental submacular surgery have also shown high VEGF levels.

In addition to being the only known endothelial cell specific mitogen, VEGF is unique among angiogenic growth factors in its ability to induce a transient increase in blood vessel permeability to macromolecules (hence its original and alternative name, vascular permeability factor, VPF) (see Dvorak et al., (1979) J. Immunol. 122:166-174; Senger et al., (1983) Science 219:983-985; Senger et al., (1986) Cancer Res. 46:5629-5632). Increased vascular permeability and the resulting deposition of plasma proteins in the extravascular space assists the new vessel formation by providing a provisional matrix for the migration of endothelial cells (Dvorak et al., (1995) Am. J. Pathol. 146:1029-1039). Hyperpermeability is indeed a characteristic feature of new vessels, including those associated with tumors.

Aspects of the invention provide VEGF variants and VEGF agonists (i.e., promoters) for use in therapy for subjects in need of treatment requiring angiogenesis or therapeutic neovascularization. Reviews of growth factor induced therapeutic angiogenesis in the heart including therapies for myocardial ischemia, end-stage coronary artery diseases and chronic peripheral arterial disease are found in J. E. Markkanen et al., Cardiovascular Research (2005) 65:656-664; B. H. Annex et al. Cardiovascular Research (2005) 65:649-655; Y. Cao et al. Cardiovascular Research (2005) 65:639-648; K. Ashara et al. Herz. (2000)25:611-622; and L. Barandon et al. Ann. Vasc. Surg. (2004)18:758-765 (the contents of each are incorporated herein by reference in their entirety).

Use of VEGF for treatment of indications where vasculogenesis is desired is found in U.S. Pat. Nos. 6,485,942 and 6,395,707 and US Patent Application Publication No.2003/0032145. Treatments using VEGF for angiogenesis and bone repair are found in R. A. D. Carano et al. Drug Discovery Today (2003) 8:980-989 and S. Bunting et al. US Patent Application Publication No. 2004/0033949 (the contents of each are incorporated herein by reference in their entirety).

Other aspects of the invention provide antagonists (i.e., inhibitors) of VEGF for use in therapy for neovascular disorders. Specific VEGF antagonists are known in the art and are described briefly in the sections that follow. Still other VEGF antagonists that are now, or that have become, available to the skilled artisan include the antibodies, aptamers, antisense oligomers, ribozymes, and RNAi compositions that may be identified and produced using practices that are routine in the art in conjunction with the teachings and guidance of the specification, including the further-provided sections appearing below.

VEGF Antagonists

Inhibition of VEGF (for example, VEGF165) is accomplished in a variety of ways. For example, a variety of VEGF antagonists that inhibit the activity or production of VEGF, including nucleic acid molecules such as aptamers, antisense RNA, ribozymes, RNAi molecules, and VEGF antibodies, are available and can be used in the methods of the present invention. Exemplary VEGF antagonists include nucleic acid ligands or aptamers of VEGF, such as those described below. A particularly useful antagonist to VEGF165 is Macugen® (pegaptanib sodium; previously referred to as EYE001 and NX1838), which is a modified, PEGylated aptamer that binds with high and specific affinity to the major soluble human VEGF isoform (see, U.S. Pat. Nos. 6,011,020; 6,051,698; and 6,147,204). The aptamer binds and inactivates VEGF in a manner similar to that of a high-affinity antibody directed towards VEGF. Another useful VEGF aptamer is EYE001 in its non-pegylated form. Alternatively, the VEGF antagonist may be, for example, an anti-VEGF antibody or antibody fragment. Accordingly, the VEGF molecule is rendered inactive by inhibiting its binding to a receptor. In addition, nucleic acid molecules such as antisense RNA, ribozymes, and RNAi molecules that inhibit VEGF expression or RNA stability at the nucleic acid level are useful antagonists in the methods and compositions of the invention. Other VEGF antagonists include peptides, proteins, cyclic peptides, and small organic compound. For example, soluble truncated forms of VEGF that bind to the VEGF receptor without concomitant signaling activity also serve as antagonists. Furthermore, the signaling activity of VEGF may be inhibited by disrupting its downstream signaling, for example, by using a number of antagonists including small molecule inhibitors of a VEGF receptor tyrosine kinase activity, as described further below.

The ability of a compound or agent to serve as a VEGF antagonist may be determined according to any number of standard methods well known in the art. For example, one of the biological activities of VEGF is to increase vascular permeability through specific binding to receptors on vascular endothelial cells. The interaction results in relaxation of the tight endothelial junctions with subsequent leakage of vascular fluid. Vascular leakage induced by VEGF can be measured in vivo by following the leakage of Evans Blue Dye from the vasculature of the guinea pig as a consequence of an intradermal injection of VEGF (Dvorak et al., in Vascular Permeability Factor/Vascular Endothelial Growth Factor, Microvascular Hyperpermeability, and Angiogenesis; (1995) Am. J. Pathol. 146:1029). Similarly, the assay can be used to measure the ability of an antagonist to block this biological activity of VEGF.

In one useful example of a vascular permeability assay, VEGF165 (20 nM-30 nM) is premixed ex vivo with Macugen® (30 nM to 1 μM) or a candidate VEGF antagonist and subsequently administered by intradermal injection into the shaved skin on the dorsum of guinea pigs. Thirty minutes following injection, the Evans Blue dye leakage around the injection sites is quantified according to standard methods by use of a computerized morphometric analysis system. A compound that inhibits VEGF-induced leakage of the indicator dye from the vasculature is taken as being a useful antagonist in the methods and compositions of the invention.

Another assay for determining whether a compound is a VEGF antagonist is the so-called corneal angiogenesis assay. In this assay, methacyrate polymer pellets containing VEGF165 (3 pmol) are implanted into the corneal stroma of rats to induce blood vessel growth into the normally avascular cornea. A candidate VEGF antagonist is then administered intravenously to the rats at doses of 1 mg/kg, 3 mg/kg, and 10 mg/kg either once or twice daily for 5 days. At the end of the treatment period, all of the individual corneas are photomicrographed. The extent to which new blood vessels develop in the corneal tissue, and their inhibition by the candidate compound, are then quantified by standardized morphometric analysis of the photomicrographs. A compound that inhibits VEGF-dependent angiogenesis in the cornea when compared to treatment with phosphate buffered saline (PBS) is taken as being a useful antagonist in the methods and compositions of the invention.

Candidate VEGF antagonists are also identified using the mouse model of retinopathy of prematurity (ROP). In one useful example, litters of 9, 8, 8, 7, and 7 mice, respectively, are left in room air or made hyperoxic and are treated intraperitoneally with phosphate buffered saline (PBS) or a candidate VEGF antagonist (for example, at 1 mg/kg, 3 mg/kg, or 10 mg/kg/day). The endpoint of the assay, outgrowth of new capillaries through the inner limiting membrane of the retina into the vitreous humor, is then assessed by microscopic identification and counting of the neovascular buds in 20 histologic sections of each eye from all of the treated and control mice. A reduction in retinal neovasculature in the treated mice relative to the untreated control is taken as identifying a useful VEGF antagonist.

In still another exemplary screening assay, candidate VEGF antagonists are identified using an in vivo human tumor xenograft assay. In this screening assay, in vivo efficacy of a candidate VEGF antagonist is tested in human tumor xenografts (A673 rhabdomyosarcoma and Wilms tumor) implanted in nude mice. Mice are then treated with the candidate VEGF antagonist (e.g., 10 mg/kg given intraperitoneally once a day following development of established tumors (200 mg)). Control groups are treated with a control agent. Candidate compounds identified as inhibiting A673 rhabdomyosarcoma tumor growth and Wilms tumor relative to the control are taken as being useful antagonists in the methods and compositions of the invention.

Additional methods of assaying for a VEGF antagonist activity are known in the art and described in further detail below.

Aspects of the invention further include VEGF antagonists known in the art as well as those supported below and any and all equivalents that are within the scope of ordinary skill to create. For example, inhibitory antibodies directed against VEGF are known in the art, e.g., those described in U.S. Pat. Nos. 6,524,583, 6,451,764 (VRP antibodies), U.S. Pat. Nos. 6,448,077, 6,416,758, 6,403,088 (to VEGF-C), U.S. Pat. No. 6,383,484 (to VEGF-D), U.S. Pat. No. 6,342,221 (anti-VEGF antibodies), U.S. Pat. Nos. 6,342,219 6,331,301 (VEGF-B antibodies), and U.S. Pat. No. 5,730,977, and PCT publications WO96/30046, WO 97/44453, and WO 98/45331, the contents of which are incorporated by reference in their entirety.

Antibodies to VEGF receptors are also known in the art, such as those described in, for example, U.S. Pat. Nos. 5,840,301, 5,874,542, 5,955,311, and 6,365,157, and PCT Publication WO 04/003211, the contents of which are incorporated by reference in their entirety.

Small molecules that block the action of VEGF by, e.g., inhibiting a VEGFR-associated tyrosine kinase activity, are known in the art, e.g., those described in U.S. Pat. Nos. 6,514,971, 6,448,277, 6,414,148, 6,362,336, 6,291,455, 6,284,751, 6,177,401, 6071,921, and 6001,885 (retinoid inhibitors of VEGF expression), the contents of each of which are incorporated by reference in their entirety.

Proteins and polypeptides that block the action of VEGF are known in the art, e.g., those described in U.S. Pat. Nos. 6,576,608, 6,559,126, 6,541,008, 6,515,105, 6,383,486 (VEGF decoy receptor), U.S. Pat. No. 6,375,929 (VEGF decoy receptor), U.S. Pat. No. 6,361,946 (VEFG peptide analog inhibitors), U.S. Pat. No. 6,348,333 (VEGF decoy receptor), U.S. Pat. No. 6,559,126 (polypeptides that bind VEGF and block binding to VEGFR), U.S. Pat. No. 6,100,071 (VEGF decoy receptor), and U.S. Pat. No. 5,952,199, the contents of each of which are incorporated by reference in their entirety.

Short interfering nucleic acids (siNA), short interfering RNA (siRNA), double-stranded RNA (dsRNA), microRNA (miRNA) and short hairpin RNA (shRNA) capable of mediating RNA interference (RNAi) against VEGF and/or VEGFR gene expression and/or activity are known in the art, for example, as disclosed in PCT Publication WO 03/070910, the contents of which is incorporated by reference in its entirety.

Antisense oligonucleotides for the inhibition of VEGF are known in the art, e.g., those described in, e.g., U.S. Pat. Nos. 5,611,135, 5,814,620, 6,399,586, 6,410,322, and 6,291,667, the contents of each of which are incorporated by reference in their entirety.

Aptamers (also known as nucleic acid ligands) for the inhibition of VEGF are known in the art, e.g., those described in, e.g., U.S. Pat. Nos. 6,762,290, 6,426,335, 6,168,778, 6,051,698, and 5,859,228, the contents of each of which are incorporated by reference in their entirety.

Antibody Antagonists

The invention, in part, includes antagonist antibodies directed against VEGF as well as its cognate receptors VEGFR. The antibody antagonists of the invention block binding of a ligand with its cognate receptor.

The antagonist antibodies of the invention include inhibitory monoclonal antibodies. Monoclonal antibodies or fragments thereof, encompass all immunoglobulin classes such as IgM, IgG, IgD, IgE, IgA, or their subclasses, such as the IgG subclasses or mixtures thereof. IgG and its subclasses are useful, such as IgG₁, IgG₂, IgG₂ₐ, IgG_{2b}, IgG₃ or IgG_{M}. The IgG subtypes IgG_{1/kappa} and IgG_{2b/kapp} are included as useful embodiments. Fragments which may be mentioned are all truncated or modified antibody fragments with one or two antigen-complementary binding sites which show high binding and neutralizing activity toward mammalian PDGF or VEGF (or their cognate receptors), such as parts of antibodies having a binding site which corresponds to the antibody and is formed by light and heavy chains, such as Fv, Fab or F(ab′)₂ fragments, or single-stranded fragments. Truncated double-stranded fragments such as Fv, Fab or F(ab′)₂ are particularly useful. These fragments can be obtained, for example, by enzymatic means by eliminating the Fc part of the antibody with enzymes such as papain or pepsin, by chemical oxidation or by genetic manipulation of the antibody genes. It is also possible and advantageous to use genetically manipulated, non-truncated fragments. The anti-VEGF antibodies or fragments thereof can be used alone or in mixtures.

The novel antibodies, antibody fragments, mixtures or derivatives thereof advantageously have a binding affinity for VEGF (or its cognate receptors) in a range from 1×10⁻⁷M to 1×10⁻¹² M, or from 1×10⁻⁸M to 1×10⁻¹¹ M, or from 1×10⁻⁹M to 5×10⁻¹⁰ M.

The antibody genes for the genetic manipulations can be isolated, for example from hybridoma cells, in a manner known to the skilled worker. For this purpose, antibody-producing cells are cultured and, when the optical density of the cells is sufficient, the mRNA is isolated from the cells in a known manner by lysing the cells with guanidinium thiocyanate, acidifying with sodium acetate, extracting with phenol, chloroform/isoamyl alcohol, precipitating with isopropanol and washing with ethanol. cDNA is then synthesized from the mRNA using reverse transcriptase. The synthesized cDNA can be inserted, directly or after genetic manipulation, for example, by site-directed mutagenesis, introduction of insertions, inversions, deletions, or base exchanges, into suitable animal, fungal, bacterial or viral vectors and be expressed in appropriate host organisms. Useful, nonlimiting bacterial or yeast vectors are pBR322, pUC18/19, pACYC184, lambda or yeast mu vectors for the cloning of the genes and expression in bacteria such as E. coli or in yeasts such as Saccharomyces cerevisiae.

Aspects of the invention furthermore relate to cells that synthesize VEGF antibodies. These include animal, fungal, bacterial cells or yeast cells after transformation as mentioned above. They are advantageously hybridoma cells or trioma cells, typically hybridoma cells. These hybridoma cells can be produced, for example, in a known manner from animals immunized with VEGF (or its cognate receptors) and isolation of their antibody-producing B cells, selecting these cells for VEGF-binding antibodies and subsequently fusing these cells to, for example, human or animal, for example, mouse myeloma cells, human lymphoblastoid cells or heterohybridoma cells (see, e.g., Koehler et al., (1975) Nature 256: 496) or by infecting these cells with appropriate viruses to produce immortalized cell lines. Hybridoma cell lines produced by fusion are useful and mouse hybridoma cell lines are particularly useful. The hybridoma cell lines of the invention secrete useful antibodies of the IgG type. The binding of the mAb antibodies of the invention bind with high affinity and reduce or neutralize the biological (e.g., angiogenic) activity of VEGF.

The invention further includes derivatives of these anti-VEGF antibodies which retain their VEGF-inhibiting activity while altering one or more other properties related to their use as a pharmaceutical agent, e.g., serum stability or efficiency of production. Examples of such anti-VEGF antibody derivatives include peptides, peptidomimetics derived from the antigen-binding regions of the antibodies, and antibodies, antibody fragments or peptides bound to solid or liquid carriers such as polyethylene glycol, glass, synthetic polymers such as polyacrylamide, polystyrene, polypropylene, polyethylene or natural polymers such as cellulose, Sepharose or agarose, or conjugates with enzymes, toxins or radioactive or nonradioactive markers such as ³H, ¹²³I, ¹²⁵I, ¹³¹I, ³²P, ³⁵S, ¹⁴C, ⁵¹Cr, ³⁶Cl, ⁵⁷Co, ⁵⁵Fe, ⁵⁹Fe, ⁹⁰Y, ^{99m}Tc, or ⁷⁵Se, or antibodies, fragments, or peptides covalently bonded to fluorescent/chemiluminescent labels such as rhodamine, fluorescein, isothiocyanate, phycoerythrin, phycocyanin, fluorescamine, metal chelates, avidin, streptavidin or biotin.

The novel antibodies, antibody fragments, mixtures, and derivatives thereof can be used directly, after drying, for example freeze drying, after attachment to the abovementioned carriers or after formulation with other pharmaceutical active and ancillary substances for producing pharmaceutical preparations. Nonlimiting examples of active and ancillary substances which may be mentioned are other antibodies, antimicrobial active substances with a microbiocidal or microbiostatic action such as antibiotics in general or sulfonamides, antitumor agents, water, buffers, salines, alcohols, fats, waxes, inert vehicles or other substances customary for parenteral products, such as amino acids, thickeners or sugars. These pharmaceutical preparations are used to control diseases, and are useful to control ocular neovascular disorders and diseases including AMD and diabetic retinopathy.

The novel antibodies, antibody fragments, mixtures or derivatives thereof can be used in therapy or diagnosis directly or can be used in therapy after coupling to solid carriers, liquid carriers, enzymes, toxins, radioactive labels, nonradioactive labels or to fluorescent/chemiluminescent labels as described above.

The human VEGF monoclonal antibodies of the present invention may be obtained by any means known in the art. For example, a mammal is immunized with human VEGF (or its cognate receptors). Purified human VEGF is commercially available (e.g., from Cell Sciences, Norwood, Mass., as well as other commercial vendors). Alternatively, human VEGF (or their cognate receptors) may be readily purified from human placental tissue. The mammal used for raising anti-human VEGF antibody is not restricted and may be a primate, a rodent (such as mouse, rat or rabbit), bovine, sheep, goat or dog.

Next, antibody-producing cells such as spleen cells are removed from the immunized animal and are fused with myeloma cells. The myeloma cells are well-known in the art (e.g., p3x63-Ag8-653, NS-0, NS-1 or P3U1 cells may be used). The cell fusion operation may be carried out by any conventional method known in the art.

The cells, after being subjected to the cell fusion operation, are then cultured in HAT selection medium so as to select hybridomas. Hybridomas which produce antihuman monoclonal antibodies are then screened. This screening may be carried out by, for example, sandwich enzyme-linked immunosorbent assay (ELISA) or the like in which the produced monoclonal antibodies are bound to the wells to which human VEGF (or its cognate receptors) is immobilized. In this case, as the secondary antibody, an antibody specific to the immunoglobulin of the immunized animal, which is labeled with an enzyme such as peroxidase, alkaline phosphatase, glucose oxidase, beta-D-galactosidase, or the like, may be employed. The label may be detected by reacting the labeling enzyme with its substrate and measuring the generated color. As the substrate, 3,3-diaminobenzidine, 2,2-diaminobis-o-dianisidine, 4-chloronaphthol, 4-aminoantipyrine, o-phenylenediamine or the like may be produced.

By the above-described operation, hybridomas which produce anti-human VEGF antibodies can be selected. The selected hybridomas are then cloned by the conventional limiting dilution method or soft agar method. If desired, the cloned hybridomas may be cultured on a large scale using a serum-containing or a serum free medium, or may be inoculated into the abdominal cavity of mice and recovered from ascites; thereby a large number of the cloned hybridomas may be obtained.

From among the selected anti-human VEGF monoclonal antibodies, those that have an ability to prevent binding and activation of the corresponding ligand/receptor pair (e.g., in a cell-based VEGF assay system (see above)) are then chosen for further analysis and manipulation. If the antibody blocks receptor/ligand binding and/or activation, it means that the monoclonal antibody tested has an ability to reduce or neutralize the VEGF activity of human VEGF. That is, the monoclonal antibody specifically recognizes and/or interferes with the critical binding site of human VEGF (or its cognate receptors).

The monoclonal antibodies herein further include hybrid and recombinant antibodies produced by splicing a variable (including hypervariable) domain of an anti-PDGF or VEGF antibody with a constant domain (e.g., “humanized” antibodies), or a light chain with a heavy chain, or a chain from one species with a chain from another species, or fusions with heterologous proteins, regardless of species of origin or immunoglobulin class or subclass designation, as well as antibody fragments (e.g., Fab, F(ab)₂, and Fv), so long as they exhibit the desired biological activity. (See, e.g., U.S. Pat. No. 4,816,567 and Mage & Lamoyi, in Monoclonal Antibody Production Techniques and Applications, pp.79-97 (Marcel Dekker, Inc.), New York (1987)).

Thus, the term “monoclonal” indicates that the character of the antibody obtained is from a substantially homogeneous population of antibodies, and is not to be construed as requiring production of the antibody by any particular method. For example, the monoclonal antibodies to be used in accordance with the present invention may be made by the hybridoma method first described by Kohler & Milstein, Nature 256:495 (1975), or may be made by recombinant DNA methods (U.S. Pat. No. 4,816,567). The “monoclonal antibodies” may also be isolated from phage libraries generated using the techniques described in McCafferty et al., Nature 348:552-554 (1990), for example.

“Humanized” forms of non-human (e.g., murine) antibodies are specific chimeric immunoglobulins, immunoglobulin chains or fragments thereof (such as Fv, Fab, Fab′, F(ab)₂ or other antigen-binding subsequences of antibodies) which contain minimal sequence derived from non-human immunoglobulin. For the most part, humanized antibodies are human immunoglobulins (recipient antibody) in which residues from the complementary determining regions (CDRs) of the recipient antibody are replaced by residues from the CDRs of a non-human species (donor antibody) such as mouse, rat or rabbit having the desired specificity, affinity and capacity. In some instances, Fv framework region (FR) residues of the human immunoglobulin are replaced by corresponding non-human FR residues. Furthermore, the humanized antibody may comprise residues that are found neither in the recipient antibody nor in the imported CDR or FR sequences. These modifications are made to further refine and optimize antibody performance. In general, the humanized antibody will comprise substantially all of at least one, and typically two, variable domains, in which all or substantially all of the CDR regions correspond to those of a non-human immunoglobulin and all or substantially all of the FR residues are those of a human immunoglobulin consensus sequence. The humanized antibody optimally also will comprise at least a portion of an inununoglobulin constant region (Fc), typically that of a human immunoglobulin.

Methods for humanizing non-human antibodies are well known in the art. 20 Generally, a humanized antibody has one or more amino acid residues introduced into it from a source which is non-human. These non-human amino acid residues are often referred to as “import” residues, which are typically taken from an “import” variable domain. Humanization can be essentially performed following the method of Winter and co-workers (Jones et al., (1986) Nature 321: 522-525; Riechmann et al., (1988) Nature 332: 323-327; and Verhoeyen et al., (1988) Science 239: 1534-1536), by substituting rodent CDRs or CDR sequences for the corresponding sequences of a human antibody.

Accordingly, such “humanized” antibodies are chimeric antibodies, wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

The choice of human variable domains, both light and heavy, to be used in making the humanized antibodies is very important to reduce antigenicity. According to the so-called “best-fit” method, the sequence of the variable domain of a rodent antibody is screened against the entire library of known human variable-domain sequences. The human sequence which is closest to that of the rodent is then accepted as the human framework (FR) for the humanized antibody (Sims et al., (1993) J. Immunol., 151:2296; and Chothia and Lesk (1987) J. Mol. Biol., 196:901). Another method uses a particular framework derived from the consensus sequence of all human antibodies of a particular subgroup of light or heavy chains. The same framework may be used for several different humanized antibodies (Carter et al., (1992) Proc. Natl.Acad. Sci. (USA), 89: 4285; and Presta et al., (1993) J. Immnol., 151:2623).

Antibodies are humanized with retention of high affinity for the antigen and other favorable biological properties. To achieve this goal, according to one useful method, humanized antibodies are prepared by a process of analysis of the parental sequences and various conceptual humanized products using three-dimensional models of the parental and humanized sequences. Three-dimensional immunoglobulin models are commonly available and are familiar to those skilled in the art. Computer programs are available which illustrate and display probable three-dimensional conformational structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind its antigen. In this way, FR residues can be selected and combined from the consensus and import sequences so that the desired antibody characteristic, such as increased affinity for the target antigen(s), is achieved. In general, the CDR residues are directly and most substantially involved in influencing antigen binding.

Human monoclonal antibodies directed against VEGF are also included in the invention. Such antibodies can be made by the hybridoma method. Human myeloma and mouse-human heteromyeloma cell lines for the production of human monoclonal antibodies have been described, for example, by Kozbor (1984) J. Immunol., 133, 3001; Brodeur, et al., Monoclonal Antibody Production Techniques and Applications, pp.51-63 (Marcel Dekker, Inc., New York, 1987); and Boerner et al., (1991) J. Immunol., 147:86-95.

Transgenic animals (e.g., mice) can be produced that are capable, upon immunization, of producing a full repertoire of human antibodies in the absence of endogenous immunoglobulin production. For example, it has been described that the homozygous deletion of the antibody heavy-chain joining region (J_{H}) gene in chimeric and germ-line mutant mice results in complete inhibition of endogenous antibody production. Transfer of the human germ-line immunoglobulin gene array in such gem-line mutant mice will result in the production of human antibodies upon antigen challenge (see, e.g., Jakobovits et al., (1993) Proc. Natl. Acad. Sci. (USA), 90: 2551; Jakobovits et al., (1993) Nature, 362:255-258; and Bruggermann et al., (1993) Year in Immuno., 7:33).

Alternatively, phage display technology (McCafferty et al., (1990) Nature, 348: 552-553) can be used to produce human antibodies and antibody fragments in vitro, from immunoglobulin variable (V) domain gene repertoires from unimmunized donors (for review see, e.g., Johnson et al., (1993) Current Opinion in Structural Biology, 3:564-571). Several sources of V-gene segments can be used for phage display. For example, Clackson et al., ((1991) Nature, 352: 624-628) isolated a diverse array of anti-oxazolone antibodies from a small random combinatorial library of V genes derived from the spleens of immunized mice. A repertoire of V genes from unimmunized human donors can be constructed and antibodies to a diverse array of antigens (including self-antigens) can be isolated essentially following the techniques described by Marks et al., ((1991) J. Mol. Biol., 222:581-597, or Griffith et al., (1993) EMBO J., 12:725-734).

In a natural immune response, antibody genes accumulate mutations at a high rate (somatic hypermutation). Some of the changes introduced will confer higher affinity, and B cells displaying high-affinity surface immunoglobulin are preferentially replicated and differentiated during subsequent antigen challenge. This natural process can be mimicked by employing the technique known as “chain shuffling” (see Marks et al., (1992) Bio.Technol., 10:779-783). In this method, the affinity of “primary” human antibodies obtained by phage display can be improved by sequentially replacing the heavy and light chain V region genes with repertoires of naturally occurring variants (repertoires) of V domain genes obtained from unimmunized donors. This technique allows the production of antibodies and antibody fragments with affinities in the nM range. A strategy for making very large phage antibody repertoires has been described by Waterhouse et al., ((1993) Nucl. Acids Res., 21:2265-2266).

Gene shuffling can also be used to derive human antibodies from rodent antibodies, where the human antibody has similar affinities and specificities to the starting rodent antibody. According to this method, which is also referred to as “epitope imprinting”, the heavy or light chain V domain gene of rodent antibodies obtained by phage display technique is replaced with a repertoire of human V domain genes, creating rodent-human chimeras. Selection on antigen results in isolation of human variable capable of restoring a functional antigen-binding site, i.e., the epitope governs (imprints) the choice of partner. When the process is repeated in order to replace the remaining rodent V domain, a human antibody is obtained (see PCT WO 93/06213, published 1 Apr. 1993). This technique provides completely human antibodies, which have no framework or CDR residues of rodent origin.

Aptamer Antagonists

The invention, in part, provides aptamer antagonists directed against VEGF (or its cognate receptors). Aptamers, also known as nucleic acid ligands, are non-naturally occurring nucleic acids that bind to and, generally, antagonize (i.e., inhibit) a pre-selected target.

Aptamers can be made by any known method of producing oligomers or oligonucleotides. Many synthesis methods are known in the art. For example, 2′-O-allyl modified oligomers that contain residual purine ribonucleotides, and bearing a suitable 3′-terminus such as an inverted thymidine residue (Ortigao et al., Antisense Research and Development, 2:129-146 (1992)) or two phosphorothioate linkages at the 3′-terminus to prevent eventual degradation by 3′-exonucleases, can be synthesized by solid phase beta-cyanoethyl phosphoramidite chemistry (Sinha et al., Nucleic Acids Res., 12:4539-4557 (1984)) on any commercially available DNA/RNA synthesizer. One method is the 2′-O-tert-butyldimethylsilyl (TBDMS) protection strategy for the ribonucleotides (Usman et al., J. Am. Chem. Soc., 109:7845-7854 (1987)), and all the required 3′-O-phosphoramidites are commercially available. In addition, aminomethylpolystyrene may be used as the support material due to its advantageous properties (McCollum and Andrus (1991) Tetrahedron Lett., 32:4069-4072). Fluorescein can be added to the 5′-end of a substrate RNA during the synthesis by using commercially available fluorescein phosphoramidites. In general, an aptamer oligomer can be synthesized using a standard RNA cycle. Upon completion of the assembly, all base labile protecting groups are removed by an eight hour treatment at 55° C. with concentrated aqueous ammonia/ethanol (3:1 v/v) in a sealed vial. The ethanol suppresses premature removal of the 2′-O-TBDMS groups that would otherwise lead to appreciable strand cleavage at the resulting ribonucleotide positions under the basic conditions of the deprotection (Usman et al., (1987) J. Am. Chem. Soc., 109:7845-7854). After lyophilization, the TBDMS protected oligomer is treated with a mixture of triethylamine trihydrofluoride/triethylamine/N-methylpyrrolidinone for 2 hours at 60° C. to afford fast and efficient removal of the silyl protecting groups under neutral conditions (see Wincott et al., (1995) Nucleic Acids Res., 23:2677-2684). The fully deprotected oligomer can then be precipitated with butanol according to the procedure of Cathala and Brunel ((1990) Nucleic Acids Res., 18:201). Purification can be performed either by denaturing polyacrylamide gel electrophoresis or by a combination of ion-exchange HPLC (Sproat et al., (1995) Nucleosides and Nucleotides, 14:255-273) and reversed phase HPLC. For use in cells, synthesized oligomers are converted to their sodium salts by precipitation with sodium perchlorate in acetone. Traces of residual salts may then be removed using small disposable gel filtration columns that are commercially available. As a final step the authenticity of the isolated oligomers may be checked by matrix assisted laser desorption mass spectrometry (Pieles et al., (1993) Nucleic Acids Res., 21:3191-3196) and by nucleoside base composition analysis.

The disclosed aptamers can also be produced through enzymatic methods, when the nucleotide subunits are available for enzymatic manipulation. For example, the RNA molecules can be made through in vitro RNA polymerase T7 reactions. They can also be made by strains of bacteria or cell lines expressing T7, and then subsequently isolated from these cells. As discussed below, the disclosed aptamers can also be expressed in cells directly using vectors and promoters.

The aptamers, like other nucleic acid molecules of the invention, may further contain chemically modified nucleotides. One issue to be addressed in the diagnostic or therapeutic use of nucleic acids is the potential rapid degration of oligonucleotides in their phosphodiester form in body fluids by intracellular and extracellular enzymes such as endonucleases and exonucleases before the desired effect is manifest. Certain chemical modifications of the nucleic acid ligand can be made to increase the in vivo stability of the nucleic acid ligand or to enhance or to mediate the delivery of the nucleic acid ligand (see, e.g., U.S. Pat. No. 5,660,985, entitled “High Affinity Nucleic Acid Ligands Containing Modified Nucleotides”) which is specifically incorporated herein by reference.

Modifications of the nucleic acid ligands contemplated in this invention include, but are not limited to, those which provide other chemical groups that incorporate additional charge, polarizability, hydrophobicity, hydrogen bonding, electrostatic interaction, and fluxionality to the nucleic acid ligand bases or to the nucleic acid ligand as a whole. Such modifications include, but are not limited to, 2′-position sugar modifications, 5-position pyrimidine modifications, 8-position purine modifications, modifications at exocyclic amines, substitution of 4-thiouridine, substitution of 5-bromo or 5-iodo-uracil; backbone modifications, phosphorothioate or alkyl phosphate modifications, methylations, unusual base-pairing combinations such as the isobases isocytidine and isoguanidine and the like. Modifications can also include 3′ and 5′ modifications such as capping or modification with sugar moieties. In some embodiments of the instant invention, the nucleic acid ligands are RNA molecules that are 2′-fluoro (2′-F) modified on the sugar moiety of pyrimidine residues.

The stability of the aptamer can be greatly increased by the introduction of such modifications and as well as by modifications and substitutions along the phosphate backbone of the RNA. In addition, a variety of modifications can be made on the nucleobases themselves which both inhibit degradation and which can increase desired nucleotide interactions or decrease undesired nucleotide interactions. Accordingly, once the sequence of an aptamer is known, modifications or substitutions can be made by the synthetic procedures described below or by procedures known to those of skill in the art.

Other nonlimiting modifications include the incorporation of modified bases (or modified nucleoside or modified nucleotides) that are variations of standard bases, sugars and/or phosphate backbone chemical structures occurring in ribonucleic (i.e., A, C, G and U) and deoxyribonucleic (i.e., A, C, G and T) acids. Included within this scope are, for example: Gm (2′-methoxyguanylic acid), Am (2′-methoxyadenylic acid), Cf (2′-fluorocytidylic acid), Uf (2′-fluorouridylic acid), Ar (riboadenylic acid). The aptamers may also include cytosine or any cytosine-related base including 5-methylcytosine, 4-acetylcytosine, 3-methylcytosine, 5-hydroxymethyl cytosine, 2-thiocytosine, 5-halocytosine (e.g., 5-fluorocytosine, 5-bromocytosine, 5-chlorocytosine, and 5-iodocytosine), 5-propynyl cytosine, 6-azocytosine, 5-trifluoromethylcytosine, N4, N4-ethanocytosine, phenoxazine cytidine, phenothiazine cytidine, carbazole cytidine or pyridoindole cytidine. The aptamer may further include guanine or any guanine-related base including 6-methylguanine, 1-methylguanine, 2,2-dimethylguanine, 2-methylguanine, 7-methylguanine, 2-propylguanine, 6-propylguanine, 8-haloguanine (e.g., 8-fluoroguanine, 8-bromoguanine, 8-chloroguanine, and 8-iodoguanine), 8-aminoguanine, 8-sulfflydrylguanine, 8-thioalkylguanine, 8-hydroxylguanine, 7-methylguanine, 8-azaguanine, 7-deazaguanine or 3-deazaguanine. The aptamer may still further include adenine or any adenine-related base including 6-methyladenine, N6-isopentenyladenine, N6-methyladenine, 1-methyladenine, 2-methyladenine, 2-methylthio-N6-isopentenyladenine, 8-haloadenine (e.g., 8-fluoroadenine, 8-bromoadenine, 8-chloroadenine, and 8-iodoadenine), 8-aminoadenine, 8-sulflhydryladenine, 8-thioalkyladenine, 8-hydroxyladenine, 7-methyladenine, 2-haloadenine (e.g., 2-fluoroadenine, 2-bromoadenine, 2-chloroadenine, and 2-iodoadenine), 2-aminoadenine, 8-azaadenine, 7-deazaadenine or 3-deazaadenine. Also included are uracil or any uracil-related base including 5-halouracil (e.g., 5-fluorouracil, 5-bromouracil, 5-chlorouracil, 5-iodouracil), 5-(carboxyhydroxylmethyl)uracil, 5-carboxymethylaminomethyl-2-thiouracil, 5-carboxymethylaminomethyluracil, dihydrouracil, 1-methylpseudouracil, 5-methoxyaminomethyl-2-thiouracil, 5′-methoxycarbonylmethyluracil, 5-methoxyuracil, 5-methyl-2-thiouracil, 2-thiouracil, 4-thiouracil, 5-methyluracil, uracil-5-oxyacetic acid methylester, uracil-5-oxyacetic acid, pseudouracil, 5-methyl-2-thiouracil, 2-thiouracil, 3-(3-amino-3-N-2-carboxypropyl)uracil, 5-methylaminomethyluracil, 5-propynyl uracil, 6-azouracil, or 4-thiouracil.

Nonlimiting examples of other modified base variants known in the art include, without limitation, those listed at 37 C.F.R. §1.822(p) (1), e.g., 4-acetylcytidine, 5-(carboxyhydroxylmethyl)uridine, 2′-methoxycytidine, 5-carboxymethylaminomethyl-2-thioridine, 5-carboxymethylaminomethyluridine, dihydrouridine, 2′-O-methylpseudouridine, b-D-galactosylqueosine, inosine, N6-isopentenyladenosine, 1-methyladenosine, 1-methylpseudouridine, 1-methylguanosine, 1-methylinosine, 2,2-dimethylguanosine, 2-methyladenosine, 2-methylguanosine, 3-methylcytidine, 5-methylcytidine, N6-methyladenosine, 7-methylguanosine, 5-methylaminomethyluridine, 5-methoxyaminomethyl-2-thiouridine, b-D-mannosylqueosine, 5-methoxycarbonylmethyluridine, 5-methoxyuridine, 2-methylthio-N6-isopentenyladenosine, N-((9-b-D-ribofuranosyl-2-methylthiopurine-6-yl)carbamoyl)threonine, N-((9-b-D-ribofuranosylpurine-6-yl)N-methyl-carbamoyl)threonine, urdine-5-oxyacetic acid methylester, uridine-5-oxyacetic acid (v), wybutoxosine, pseudouridine, queosine, 2-thiocytidine, 5-methyl-2-thiouridine, 2-thiouridine, 4-thiouridine, 5-methyluridine, N-((9-b-D-ribofuranosylpurine-6-yl)carbamoyl)threonine, 2′-O-methyl-5-methyluridine, 2′-O-methyluridine, wybutosine, 3-(3-amino-3-carboxypropyl)uridine.

Also included are the modified nucleobases described in U.S. Pat. Nos. 3,687,808, 3,687,808, 4,845,205, 5,130,302, 5,134,066, 5,175,273, 5,367,066, 5,432,272, 5,457,187, 5,459,255, 5,484,908, 5,502,177, 5,525,711, 5,552,540, 5,587,469, 5,594,121, 5,596,091, 5,614,617, 5,645,985, 5,830,653, 5,763,588, 6,005,096, and 5,681,941. Examples of modified nucleoside and nucleotide sugar backbone variants known in the art include, without limitation, those having, e.g., 2′ ribosyl substituents such as F, SH, SCH3, OCN, Cl, Br, CN, CF3, OCF3, SOCH3, SO2, CH3, ONO2, NO2, N3, NH2, OCH2CH2OCH3, O(CH2)2ON(CH3)2, OCH2OCH2N(CH3)2, O(C1-10 alkyl), alkenyl), O(C2-10 alkynyl), S(C1-10 alkyl), S(C2-10 alkenyl), S(C2-10 alkynyl), NH(C1-10 alkyl), NH(C2-10 alkenyl), NH(C2-10 alkynyl), and O-alkyl-O-alkyl. Desirable 2′ ribosyl substituents include 2′-methoxy (2′-OCH3), 2′-aminopropoxy (2′-OCH2CH2CH2NH2), 2′-allyl (2′-CH2-CH═CH2), 2′-O-allyl (2′-O—CH2-CH═CH2), 2′-amino (2′-NH2), and 2′-fluoro (2′-F). The 2′-substituent may be in the arabino (up) position or ribo (down) position.

The aptamers of the invention may be made up of nucleotides and/or nucleotide analogs such as described above, or a combination of both, or are oligonucleotide analogs. The aptamers of the invention may contain nucleotide analogs at positions which do not effect the function of the oligomer to bind VEGF (or its cognate receptors).

There are several techniques that can be adapted for refinement or strengthening of the nucleic acid Ligands binding to a particular target molecule or the selection of additional aptamers. One technique, generally referred to as “in vitro genetics” (see Szostak (1992) TIBS, 19:89), involves isolation of aptamer antagonists by selection from a pool of random sequences. The pool of nucleic acid molecules from which the disclosed aptamers may be isolated may include invariant sequences flanking a variable sequence of approximately twenty to forty nucleotides. This method has been termed Selective Evolution of Ligands by EXponential Enrichment (SELEX). Compositions and methods for generating aptamer antagonists of the invention by SELEX and related methods are known in the art and taught in, for example, U.S. Pat. No. 5,475,096 entitled “Nucleic Acid Ligands,” and U.S. Pat. No. 5,270,163, entitled “Methods for Identifying Nucleic Acid Ligands,” each of which is specifically incorporated by reference herein in its entirety. The SELEX process in general, and VEGF aptamers and formulations in particular, are further described in, e.g., U.S. Pat. Nos. 5,668,264, 5,696,249, 5,670,637, 5,674,685, 5,723,594, 5,756,291, 5,811,533, 5,817,785, 5,958,691, 6,011,020, 6,051,698, 6,147,204, 6,168,778, 6,207,816, 6,229,002, 6,426,335, and 6,582,918, the contents of each of which is specifically incorporated by reference herein.

Briefly, the SELEX method involves selection from a mixture of candidate oligonucleotides and step-wise iterations of binding to a selected target, partitioning and amplification, using the same general selection scheme, to achieve virtually any desired criterion of binding affinity and selectivity. Starting from a mixture of nucleic acids, typically comprising a segment of randomized sequence, the SELEX method includes steps of contacting the mixture with the target under conditions favorable for binding, partitioning unbound nucleic acids from those nucleic acids which have bound specifically to target molecules, dissociating the nucleic acid-target complexes, amplifying the nucleic acids dissociated from the nucleic acid-target complexes to yield a ligand-enriched mixture of nucleic acids, then reiterating the steps of binding, partitioning, dissociating and amplifying through as many cycles as desired to yield highly specific high affinity nucleic acid ligands to the target molecule.

The basic SELEX method has been modified to achieve a number of specific objectives. For example, U.S. Pat. No. 5,707,796, entitled “Method for Selecting Nucleic Acids on the Basis of Structure,” describes the use of the SELEX process in conjunction with gel electrophoresis to select nucleic acid molecules with specific structural characteristics, such as bent DNA. U.S. Pat. No. 5,763,177 entitled “Systematic Evolution of Ligands by Exponential Enrichment: Photoselection of Nucleic Acid Ligands and Solution SELEX” describe a SELEX based method for selecting nucleic acid ligands containing photoreactive groups capable of binding and/or photocrosslinking to and/or photoinactivating a target molecule. U.S. Pat. No. 5,580,737 entitled “High-Affinity Nucleic Acid Ligands That Discriminate Between Theophylline and Caffeine,” describes a method for identifying highly specific nucleic acid ligands able to discriminate between closely related molecules, which can be non-peptidic, termed Counter-SELEX. U.S. Pat. No. 5,567,588 entitled “Systematic Evolution of Ligands by EXponential Enrichment: Solution SELEX,” describes a SELEX-based method which achieves highly efficient partitioning between oligonucleotides having high and low affinity for a target molecule.

The SELEX method encompasses the identification of high-affinity nucleic acid ligands containing modified nucleotides conferring improved characteristics on the ligand, such as improved in vivo stability or improved delivery characteristics. Nonlimiting examples of such modifications include chemical substitutions at the ribose and/or phosphate and/or base positions. SELEX process-identified nucleic acid ligands containing modified nucleotides are described in U.S. Pat. No. 5,660,985 entitled “High Affinity Nucleic Acid Ligands Containing Modified Nucleotides,” that describes oligonucleotides containing nucleotide derivatives chemically modified at the 5- and 2′-positions of pyrimidines. U.S. Pat. No. 5,580,737, supra, describes highly specific nucleic acid ligands containing one or more nucleotides modified with 2′-amino (2′-NH₂), 2′-fluoro (2′-F), and/or 2′-O-methyl (2′-OMe). U.S. application Ser. No. 08/264,029, filed Jun. 22, 1994, entitled “Novel Method of Preparation of Known and Novel 2′ Modified Nucleosides by Intramolecular Nucleophilic Displacement,” now abandoned, describes oligonucleotides containing various 2′-modified pyrimidines.

The SELEX method encompasses combining selected oligonucleotides with other selected oligonucleotides and non-oligonucleotide functional units as described in U.S. Pat. No. 5,637,459 entitled “Systematic Evolution of Ligands by EXponential Enrichment: Chimeric SELEX,” and U.S. Pat. No. 5,683,867 entitled “Systematic Evolution of Ligands by EXponential Enrichment: Blended SELEX,” respectively. These patents allow for the combination of the broad array of shapes and other properties, and the efficient amplification and replication properties, of oligonucleotides with the desirable properties of other molecules.

The SELEX method further encompasses combining selected nucleic acid ligands with lipophilic compounds or non-immunogenic, high molecular weight compounds in a diagnostic or therapeutic complex as described in U.S. Pat. No. 6,011,020, entitled “Nucleic Acid Ligand Complexes,” which is specifically incorporated by reference herein in their entirety.

The aptamer antagonists can also be refined through the use of computer modeling techniques. Examples of molecular modeling systems are the CHARMm and QUANTA programs, Polygen Corporation (Waltham, Mass.). CHARMm performs the energy minimization and molecular dynamics functions. QUANTA performs the construction, graphic modeling and analysis of molecular structure. QUANTA allows interactive construction, modification, visualization, and analysis of the behavior of molecules with each other. These applications can be adapted to define and display the secondary structure of RNA and DNA molecules.

Aptamers with these various modifications can then be tested for function using any suitable assay for the VEGF function of interest, such as a VEGF cell-based proliferation activity assay.

The modifications can be pre- or post-SELEX process modifications. Pre-SELEX process modifications yield nucleic acid ligands with both specificity for their SELEX target and improved in vivo stability. Post-SELEX process modifications made to 2′-OH nucleic acid ligands can result in improved in vivo stability without adversely affecting the binding capacity of the nucleic acid ligand.

Other modifications useful for producing aptamers of the invention are known to one of ordinary skill in the art. Such modifications may be made post-SELEX process (modification of previously identified unmodified ligands) or by incorporation into the SELEX process.

It has been observed that aptamers, or nucleic acid ligands, in general, and VEGF aptamers in particular, are most stable, and therefore efficacious when 5′-capped and 3′-capped in a manner which decreases susceptibility to exonucleases and increases overall stability. (See Adamis, A. P. et al., published application No. WO 2005/014814, which is hereby incorporated by reference in its entirety). Accordingly, the invention, in part, is based in one embodiment, upon the capping of aptamers in general, and anti-VEGF aptamers in particular, with a 5′-5′ inverted nucleoside cap structure at the 5′ end and a 3′-3′ inverted nucleoside cap structure at the 3′ end. Thus, the invention, in part, provides anti-VEGF and/or anti-PDGF aptamers, i.e., nucleic acid ligands, that are capped at the 5′end with a 5′-5-inverted nucleoside cap and at the 3′ end with a 3′-3′ inverted nucleoside cap.

Certain particularly useful aptamers of the invention are anti-VEGF aptamer compositions, including, but not limited to, those having both 5′-5′ and 3′-3′ inverted nucleotide cap structures at their ends. Such anti-VEGF capped aptamers may be RNA aptamers, DNA aptamers or aptamers having a mixed (i.e., both RNA and DNA) composition. Suitable anti-VEGF aptamer sequences of the invention include the nucleotide sequence GAAGAAUUGG (SEQ ID NO: 34); or the nucleotide sequence UUGGACGC (SEQ ID NO: 35); or the nucleotide sequence GUGAAUGC (SEQ ID NO: 36). Particularly useful are capped anti-VEGF aptamers of the invention have the sequence:

(SEQ ID NO:37)

X-5′-5′-CGGAAUCAGUGAAUGCUUAUACAUCCG-3′-3′-X

where each C, G, A, and U represents, respectively, the naturally-occurring nucleotides cytidine, guanidine, adenine, and uridine, or modified nucleotides corresponding thereto; X-5′-5′ is an inverted nucleotide capping the 5′ terminus of the aptamer; 3′-3′-X is an inverted nucleotide capping the 3′ terminus of the aptamer; and the remaining nucleotides or modified nucleotides are sequentially linked via 5′-3′ phosphodiester linkages. In some embodiments, each of the nucleotides of the capped anti-VEGF aptamer, individually carries a 2′ ribosyl substitution, such as —OH (which is standard for ribonucleic acids (RNAs)), or —H (which is standard for deoxyribonucleic acids (DNAs)). In other embodiments the 2′ ribosyl position is substituted with an O(C₁₋₁₀ alkyl), an O(C₁₋₁₀ alkenyl), a F, an N₃, or an NH₂ substituent.

In a still more particular non-limiting example, the 5′-5′ capped anti-VEGF aptamer may have the structure:

| | |
|---|---|
| T_{d}-5′-5′C_{f}GₘGₘAᵣAᵣU_{f}C_{f}AₘGₘU_{f}GₘAₘAₘ | (SEQ ID NO:38) |
| U_{f}GₘC_{f}U_{f}U_{f}AₘU_{f}AₘC_{f}AₘU_{f}C_{f}C_{f}Gₘ 3′-3′- | |
| T_{d} | |

where “Gₘ” represents 2′-methoxyguanylic acid, “Aₘ” represents 2′-methoxyadenylic acid, “C_{f}” represents 2′-fluorocytidylic acid, “U_{f}” represents 2′-fluorouridylic acid, “Aᵣ” represents riboadenylic acid, and “T_{d}” represents deoxyribothymidylic acid.

Still other related compounds for inhibition or activation of VEGFR are available by screening novel compounds for their effect on the receptor tyrosine kinase activity of interest using a convention assay. Effective inhibition or activation by a candidate VEGFR small molecule organic inhibitor or activator can be monitored using a cell-based assay system as well as other assay systems known in the art.

For example, one test for activity against VEGF-receptor tyrosine kinase is as follows. The test is conducted using Flt-1 VEGF-receptor tyrosine kinase. The detailed procedure is as follows: 30 μl kinase solution (10 ng of the kinase domain of Flt-1 (see Shibuya, et al., (1990) Oncogene, 5: 519-24) in 20 mM Tris.HCl pH 7.5,3 mM manganese dichloride (MnCl₂), 3 mM magnesium chloride (MgCl₂), 10 μM sodium vanadate, 0.25 mg/ml polyethylenglycol (PEG) 20000, 1 mM dithiothreitol and 3 ug/.mu.l poly(Glu,Tyr) 4:1 (Sigma, Buchs, Switzerland), 8 uM [³³P]-ATP (0.2 uCi), 1% dimethyl sulfoxide, and 0 to 100 μM of the compound to be tested are incubated together for 10 minutes at room temperature. The reaction is then terminated by the addition of 10 μl 0.25 M ethylenediaminetetraacetate (EDTA) pH 7. Using a multichannel dispenser (LAB SYSTEMS, USA), an aliquot of 20 μl is applied to a PVDF (=polyvinyl difluoride) Immobilon P membrane (Millipore, USA), through a microtiter filter manifold and connected to a vacuum. Following complete elimination of the liquid, the membrane is washed 4 times successively in a bath containing 0.5% phosphoric acid (H₃ PO₄) and once with ethanol, incubated for 10 minutes each time while shaking, then mounted in a Hewlett Packard TopCount Manifold and the radioactivity measured after the addition of 10 μl Microscint.RTM. (beta-scintillation counter liquid). IC₅₀-values are determined by linear regression analysis of the percentages for the inhibition of each compound in three concentrations (as a rule 0.01 μmol, 0.1 μmol, and 1 μmol). The IC₅₀ -values of active tyrosine inhibitor compounds may be in the range of 0.01 μM to 100 μM.

Furthermore, inhibition or activation of a VEGF-induced VEGFR tyrosine kinase/autophosphorylation activity can be confirmed with a further experiment on cells. Briefly, transfected CHO cells, which permanently express human VEGF receptor (VEGFR/KDR), are seeded in complete culture medium (with 10% fetal call serum (FCS) in 6-well cell-culture plates and incubated at 37° C. under 5% CO₂ until they show about 80% confluency. The compounds to be tested are then diluted in culture medium (without FCS, with 0.1% bovine serum albumin) and added to the cells. (Controls comprise medium without test compounds). After a two hour incubation at 37° C., recombinant VEGF is added; the final VEGF concentration is 20 ng/ml). After a further five minutes incubation at 37° C., the cells are washed twice with ice-cold PBS) and immediately lysed in 100 μl lysis buffer per well. The lysates are then centrifuged to remove the cell nuclei, and the protein concentrations of the supernatants are determined using a commercial protein assay (BIORAD). The lysates can then either be immediately used or, if necessary, stored at −200° C.

A sandwich ELISA is then carried out to measure the KDR-receptor phosphorylation: a monoclonal antibody to KDR is immobilized on black ELISA plates (OptiPlateTM, HTRF-96 from Packard). The plates are then washed and the remaining free protein-binding sites are saturated with 1% BSA in PBS. The cell lysates (20 μg protein per well) are then incubated in these plates overnight at 4° C. together with an antiphosphotyrosine antibody coupled with alkaline phosphatase (e.g., PY20:AP from Transduction Laboratories, Lexington, Ky.). The plates are washed again and the binding of the antiphosphotyrosine antibody to the captured phosphorylated receptor is then demonstrated using a luminescent AP substrate (CDP-Star, ready to use, with Emerald II; Applied-Biosystems TROPIX Bedford, Mass.). The luminescence is measured, e.g., in a Packard Top Count Microplate Scintillation Counter. The difference between the signal of the positive control (stimulated with VEGF) and that of the negative control (not stimulated with VEGF) corresponds to VEGF-induced KDR-receptor phosphorylation (=100%). The activity of the tested substances is calculated as percent inhibition of VEGF-induced KDR-receptor phosphorylation, wherein the concentration of substance that induces half the maximum inhibition is defined as the ED₅₀ (effective dose for 50% inhibition). Active tyrosine inhibitor compound have ED₅₀ values in the range of 0.001 μM to 6 μM, or 0.005 μM to 0.5 μM.

Pharmaceutical Formulations and Therapeutic Administration

The VEGF antagonist compositions of the invention are useful in the treatment of a neovascular disorder, including psoriasis, rheumatoid arthritis, and ocular neovascular disorders. Of particular interest are therapies using a VEGF antagonist to suppress an ocular neovascular disorder such as macular degeneration or diabetic retinopathy. Accordingly, once a patient has been diagnosed to be at risk at developing or having a neovascular disorder, the patient is treated by administration of a VEGF antagonist in order to block respectively the negative effects of VEGF, thereby suppressing the development of a neovascular disorder and alleviating deleterious effects associated with neovascularization. The practice of the methods according to the present invention does not result in comeal edema. As is discussed above, a wide variety of VEGF antagonists may be used in the present invention.

Administration of the compositions of the present invention may be administered by any suitable means that results in a concentration that is effective for the treatment of a neovascular disorder. Each composition, for example, may be admixed with a suitable carrier substance, and is generally present in an amount of 1-95% by weight of the total weight of the composition. The composition may be provided in a dosage form that is suitable for ophthalmic, oral, parenteral (e.g., intravenous, intramuscular, subcutaneous), rectal, transdermal, nasal, or inhalant administration. Accordingly, the composition may be in form of, e.g., tablets, capsules, pills, powders, granulates, suspensions, emulsions, solutions, gels including hydrogels, pastes, ointments, creams, plasters, delivery devices, suppositories, enemas, injectables, implants, sprays, or aerosols. The pharmaceutical compositions containing a single antagonist or two or more antagonists may be formulated according to conventional pharmaceutical practice (see, e.g., Remington: The Science and Practice of Pharmacy, (20th ed.) ed. A. R. Gennaro, 2000, Lippincott Williams & Wilkins, Philadelphia, Pa. and Encyclopedia of Pharmaceutical Technology, eds., J. Swarbrick and J. C. Boylan, 1988-2002, Marcel Dekker, New York).

The compositions of the present invention are, in one useful aspect, administered parenterally (e.g., by intramuscular, intraperitoneal, intravenous, intraocular, intravitreal, retro-bulbar, subconjunctival, subtenon or subcutaneous injection or implant) or systemically. Formulations for parenteral or systemic administration include sterile aqueous or non-aqueous solutions, suspensions, or emulsions. A variety of aqueous carriers can be used, e.g., water, buffered water, saline, and the like. Nonlimiting examples of other suitable vehicles include polypropylene glycol, polyethylene glycol, vegetable oils, gelatin, hydrogels, hydrogenated naphalenes, and injectable organic esters, such as ethyl oleate. Such formulations may also contain auxiliary substances, such as preserving, wetting, buffering, emulsifying, and/or dispersing agents. Biocompatible, biodegradable lactide polymer, lactide/glycolide copolymer, or polyoxyethylene-polyoxypropylene copolymers may be used to control the release of the active ingredients.

Alternatively, the compositions of the present invention can be administered by oral ingestion. Compositions intended for oral use can be prepared in solid or liquid forms, according to any method known to the art for the manufacture of pharmaceutical compositions.

Solid dosage forms for oral administration include capsules, tablets, pills, powders, and granules. Generally, these pharmaceutical preparations contain active ingredients admixed with non-toxic pharmaceutically acceptable excipients. These may include, for example, inert diluents, such as calcium carbonate, sodium carbonate, lactose, sucrose, glucose, mannitol, cellulose, starch, calcium phosphate, sodium phosphate, kaolin and the like. Binding agents, buffering agents, and/or lubricating agents (e.g., magnesium stearate) may also be used. Tablets and pills can additionally be prepared with enteric coatings. The compositions may optionally contain sweetening, flavoring, coloring, perfuming, and preserving agents in order to provide a more palatable preparation.

The compositions of the present invention may be administered intraocularly by intravitreal injection into the eye as well as subconjunctival and subtenon injections. Other routes of administration include transcleral, retro bulbar, intraperoteneal, intramuscular, and intravenous. Alternatively, a composition may be delivered using a drug delivery device or an intraocular implant (see below).

Liquid dosage forms for oral administration include pharmaceutically acceptable emulsions, solutions, suspensions, syrups, and soft gelatin capsules. These forms contain inert diluents commonly used in the art, such as, but not limited to, water or an oil medium, and can also include adjuvants, such as wetting agents, emulsifying agents, and suspending agents.

In some instances, the compositions of the present invention can also be administered topically, for example, by patch or by direct application to a region, such as the epidermis or the eye, susceptible to or affected by an ocular disorder, or by iontophoresis.

Formulations for ophthalmic use include tablets containing the active ingredient(s) in a mixture with non-toxic pharmaceutically acceptable excipients. These excipients may be, for example, inert diluents or fillers (e.g., sucrose and sorbitol), lubricating agents, glidants, and antiadhesives (e.g., magnesium stearate, zinc stearate, stearic acid, silicas, hydrogenated vegetable oils, or talc).

Generally, each formulation is administered in an amount sufficient to suppress or reduce or eliminate a deleterious effect or a symptom of a disorder. The amount of an active ingredient that is combined with the carrier materials to produce a single dosage will vary depending upon the subject being treated and the particular mode of administration.

The dosage of each formulation depends on several factors including the severity of the condition, whether the condition is to be treated or prevented, and the age, weight, and health of the person to be treated. Additionally, pharmacogenomic (the effect of genotype on the pharmacokinetic, pharmacodynamic or efficacy profile of a therapeutic) information about a particular patient may affect dosage used. Furthermore, one skilled in the art will appreciate that the exact individual dosages may be adjusted somewhat depending on a variety of factors, including the specific composition being administered, the time of administration, the route of administration, the nature of the formulation, the rate of excretion, the particular neovascular disorder being treated, the severity of the disorder, and the anatomical location of the neovascular disorder (for example, the eye versus the body cavity). Wide variations in the needed dosage are to be expected in view of the differing efficiencies of the various routes of administration. For instance, oral administration generally would be expected to require higher dosage levels than administration by intravenous or intravitreal injection. Variations in these dosage levels can be adjusted using standard empirical routines for optimization, which are well-known in the art. The precise therapeutically effective dosage levels and patterns are typically determined by the attending physician such as an ophthalmologist in consideration of the above-identified factors.

Generally, when orally administered to a human, the dosage of the compositions of the present invention is normally about 0.001 mg to about 200 mg per day, about 1 mg to 100 mg per day, or about 5 mg to about 50 mg per day. Dosages up to about 200 mg per day may be necessary. For administration by injection, the dosage is normally about 0.1 mg to about 250 mg per day, about 1 mg to about 20 mg per day, or about 3 mg to about 5 mg per day. Injections may be given up to about four times daily. Generally, when parenterally or systemically administered to a human, the dosage is normally about 0.1 mg to about 1500 mg per day, or about 0.5 mg to 10 about mg per day, or about 0.5 mg to about 5 mg per day. Dosages up to about 3000 mg per day may be necessary.

When ophthalmologically administered to a human, the dosage is normally about 0.15 mg to about 3.0 mg per day, or at about 0.3 mg to about 3.0 mg per day, or at about 0.1 mg to 1.0 mg per day.

Administration of a drug can, independently, be one to four times daily for one day to one year, and may even be for the life of the patient. Chronic, long-term administration will be indicated in many cases. The dosage may be administered as a single dose or divided into multiple doses. In general, the desired dosage should be administered at set intervals for a prolonged period, usually at least over several weeks, although longer periods of administration of several months or more may be needed.

In addition to treating pre-existing disorders, the therapy that includes a VEGF antagonist can be administered prophylactically in order to prevent or slow the onset of these disorders. In prophylactic applications, the VEGF antagonists is administered to a patient susceptible to or otherwise at risk of a particular neovascular disorder. The precise timing of the administration and amounts that are administered depend on various factors such as the patient's state of health, weight, etc.

Pharmaceutical compositions according to the invention may be formulated to release the compositions of the present invention substantially immediately upon administration or at any predetermined time period after administration, using controlled release formulations. For example, a pharmaceutical composition that includes at least one composition of the present invention may be provided in sustained release compositions. The use of immediate or sustained release compositions depends on the nature of the condition being treated. If the condition consists of an acute or over-acute disorder, treatment with an immediate release form will be typically utilized over a prolonged release composition. For certain preventative or long-term treatments, a sustained released composition may also be appropriate.

Administration of the compositions in controlled release formulations is useful where the composition, either alone or in combination, has (i) a narrow therapeutic index (e.g., the difference between the plasma concentration leading to harmful side effects or toxic reactions and the plasma concentration leading to a therapeutic effect is small; generally, the therapeutic index, TI, is defined as the ratio of median lethal dose (LD₅₀) to median effective dose (ED₅₀)); (ii) a narrow absorption window in the gastro-intestinal tract; or (iii) a short biological half-life, so that frequent dosing during a day is required in order to sustain the plasma level at a therapeutic level.

Many strategies can be pursued to obtain controlled release in which the rate of release outweighs the rate of degradation or metabolism of the therapeutic. For example, controlled release can be obtained by the appropriate selection of formulation parameters and ingredients, including, e.g., appropriate controlled release compositions and coatings. Examples include single or multiple unit tablet or capsule compositions, oil solutions, suspensions, emulsions, microcapsules, microspheres, nanoparticles, patches, and liposomes. Methods for preparing such sustained or controlled release formulations are well known in the art.

Pharmaceutical compositions that include a composition of the present invention may also be delivered using a drug delivery device such as an implant. Such implants may be biodegradable and/or biocompatible implants, or may be non-biodegradable implants. The implants may be permeable or impermeable to the active agent.

Ophthalmic drug delivery devices may be inserted into a chamber of the eye, such as the anterior or posterior chambers or may be implanted in or on the scelra, choroidal space, or an avascularized region exterior to the vitreous. In one embodiment, the implant may be positioned over an avascular region, such as on the sclera, so as to allow for transcleral diffusion of the drug to the desired site of treatment, e.g., the intraocular space and macula of the eye. Furthermore, the site of transcleral diffusion may be proximity to a site of neovascularization such as a site proximal to the macula.

The invention optionally relates to combining separate pharmaceutical compositions in a pharmaceutical pack. The combination of the invention is therefore optionally provided as components of a pharmaceutical pack. The components can be formulated together or separately and in individual dosage amounts.

The compositions of the invention are also useful when formulated as salts.

Effectiveness

Suppression of a neovascular disorder is evaluated by any accepted method of measuring whether angiogenesis is slowed or diminished. This includes direct observation and indirect evaluation such as by evaluating subjective symptoms or objective physiological indicators. Treatment efficacy, for example, may be evaluated based on the prevention or reversal of neovascularization, microangiopathy, vascular leakage or vascular edema or any combination thereof. Treatment efficacy for evaluating suppression of an ocular neovascular disorder may also be defined in terms of stabilizing or improving visual acuity.

In determining the effectiveness of a particular therapy in treating or preventing an ocular neovascular disorder, patients may also be clinically evaluated by an ophthalmologist several days after injection and at least one-month later just prior to the next injection. ETDRS visual acuities, kodachrome photography, and fluorescein angiography are also performed monthly for the first 4 months as required by the ophthalmologist.

For example, in order to assess the effectiveness of VEGF antagonist therapy to treat ocular neovascularization, studies are conducted involving the administration of either single or multiple intravitreal injections of a VEGF-A aptamer (for example, a PEGylated form of EYE001) in patients suffering from subfoveal choroidal neovascularization secondary to age-related macular degeneration according to standard methods well known in the ophthalmologic arts. In one working study, patients with subfoveal choroidal neovascularization (CNV) secondary to age-related macular degeneration (AMD) receive a single intravitreal injection of a VEGF variant, or a VEGF aptamer. Effectiveness of the composition is monitored, for example, by ophthalmic evaluation. Patients showing stable or improved vision three months after treatment, for example, demonstrating a 3-line or greater improvement in vision on the ETDRS chart, are taken as receiving an effective dosage of a VEGF variant or a VEGF aptamer that suppresses an ocular neovascular disorder.

### EXAMPLES

The following examples serve to illustrate certain useful embodiments and aspects of the present invention and are not to be construed as limiting the scope thereof. Alternative materials and methods can be utilized to obtain similar results.

### Example 1

Site-Directed Mutagenesis

Alanine substitutions were introduced into exon 7 (Pro116-Cys160) of full-length VEGF164 by PCR using the QuikChange™ Multi Site-directed Mutagenesis Kit (Stratagene).

Oligonucleotide primers containing the desired mutation flanked by unmodified nucleotide sequence were synthesized and purified by HPLC and ethanol precipitation. They were designed to bind to adjacent sequences or to separate regions on the same strand of the template plasmid. Primers were usually 32-43 bp in length and were 5′-phosphorylated for better mutagenesis efficiency. They had a minimum GC content of 40% with a melting temperature (Tₘ) of ≧75° C. and terminate in one or more C or G bases at the 3′-end. Reactions were carried out in the appropriate buffer in 25 μL using 100 ng of each primer, 50 ng double-stranded DNA template, 1 μL dNTP mix, and 1 μL of Pfu Turbo DNA polymerase enzyme blend (Stratagene).

The following PCR conditions were used:

| | | |
|---|---|---|
| Segment 1 | 1 cycle | denaturation at 95° C. for 1 min |
| Segment 2 | 30 cycles | denaturation at 95° C. for 1 min |
| | | annealing at 55° C. for 1 min |
| | | extension at 65° C. for 2 min/kb of plasmid |
| | | length |

The reaction was placed on ice for 2 min, before adding 10 U of DpnI-restriction enzyme for 1 hour at 37° to digest the parental (nonmutated) DNA template. 1.5 μl of the DpnI-treated DNA was transformed into XL 10-Gold ultracompetent cells (Stratagene) by incubating at 42° C. for 30 seconds. SOC medium was added and the tubes were then incubated at 37, the reaction was incubated at 37° C. for 1 hour. Appropriate volumes of each transformation reaction was plated on low salt LB agar plates containing 25 μg/mL Zeocin. The mutagenesis efficiency of a control plasmid was determined as a positive control in each experiment.

The following mutant oligonucleotides were used as primers For generating VEGF164 heparin binding domain mutants:

| | |
|---|---|
| R13/14A | |
| 5′-TGTGAGCCTTGTTCAGAGGCGGCAAAGCATTTG | (SEQ ID NO:39) |
| TTTGTCC-3′; | |
| A13R | |
| 5′-GAGCCTTGTTCAGAGCGGGCAAAGCATTTGTTT | (SEQ ID NO:40) |
| GTCC-3′; | |
| R49A | |
| 5′-AACGAACGTACTTGCGCATGTGACAAGCCGAG | (SEQ ID NO:41) |
| G-3′; | |
| A13R (R13A reverse) | |
| 5′-GAGCCTTGTTCAGAGCGGGCAAAGCATTTGTTT | (SEQ ID NO:39) |
| GTCC-3′; | |
| Q20/Q23A | |
| 5′-CATTTGTTTGTCGCAGATCCGGCGACGTGTAAA | (SEQ ID NO:42) |
| TGTTCC-3′; | |
| K26A | |
| 5′-GTCCAAGATCCGCAGACGTGTGCATGTTCCTGC | (SEQ ID NO:43) |
| AA-3′; | |
| K30A | |
| 5′-CGTGTAAATGTTCCTGCGCAAACACAGACTC | (SEQ ID NO:44) |
| G-3′; | |
| R35/R39A | |
| 5′-AACACAGACTCGGCTTGCAAGGCGGCGCAGCTT | (SEQ ID NO:45) |
| GAGTTAAACG-3′; | |
| R46/R49A | |
| 5′-TGAGTTAAACGAAGCTACTTGCGCATGTGACAA | (SEQ ID NO:46) |
| GCCGAGG-3′. | |

Nucleotides in bold indicate mutations.
All mutations were confirmed by DNA sequencing.

A pPICZaC-VEGF164 expression plasmid was used as the template to generate the triple mutant (R13A/R14A/R49A) in one step. The double mutant (R14/R49A) was generated in the context of the triple mutant by reversing the R13→A13 mutation.

| | |
|---|---|
| Double Mutant (R14/R49A): | |
| APMA EGGGQNHHEV VKFMDVYQRS | (Seq. ID No. 23) |
| YCHPIETLVD IFQEYPDEIE YIFKPSCVPL | |
| MRCGGCCNDE GLECVPTEES NITMQIMRIK | |
| PHQGQHIGEM SFLQHNKCEC RPKKDRARQE | |
| NPCGPC SERAKHLFVQ DPQTCKCSCK | |
| NTDSRCKARQ LELNERTCAC DKPRR. | |
| Triple Mutant (R13A/R14A/R49A): | |
| APMA EGGGQNHHEV VKFMDVYQRS | (Seq. ID No. 24) |
| YCHPIETLVD IFQEYPDEIE YIFKPSCVPL | |
| MRCGGCCNDE GLECVPTEES NITMQIMRIK | |
| PHQGQHIGEM SFLQHNKCEC RPKKDRARQE | |
| NPCGPC SEAAKHLFVQ DPQTCKCSCK | |
| NTDSRCKARQ LELNERTCAC DKPRR. | |

Discussion

Applicants describe the successful use of alanine scanning mutagenesis to define the interactions of heparin-binding proteins with heparin. Further, applicants identified residues that contribute to the interaction of VEGF164 with heparin by employing in vitro heparin binding assays.

The availability of NMR structural data on the heparin binding domain fragment of VEGF164 (VEGF55) has helped design and rationalize a mutagenesis strategy aimed at defining important residues involved in the interaction of VEGF164 with heparin (Lee et al. PNAS, (2005) Vol. 102, 18902-18907, the contents of which is incorporated herein by reference in its entirety). Fairbrother, W.J. et al. suggested that clusters of basic amino acid side-chains on one side of the carboxy-terminal subdomain and an amino-terminal loop-region may represent a heparin binding site (Fairbrother, W. J., et al., Structure, 1998. 6(5): p. 637-48 the contents of which is incorporated herein by reference in its entirety). To test this hypothesis, 8 basic residues belonging to exon 7 encoded region were selected and changed to alanine either individually or in combination by site-directed mutagenesis. Ten different VEGF164 mutant proteins were produced in the methylotrophic yeast Pichia Pastoris and purified to homogeneity. Unlike bacterial expression systems, proteins produced in this organism do not require refolding. In addition, protein processing and posttranslational modification more closely resemble that of higher eukaryotic organisms. Each of the recombinant mutants was found to be similar to wildtype VEGF164, with regard to secretion, yield and the ability to form disulfide-linked homodimers. Moreover, the biological activity of each mutant, as assessed in an endothelial cell-based assay, was confirmed to be as potent as native VEGF164. These findings indicate that the proteins were folded and that the mutations had no damaging effect on their structural integrity, in agreement with the fact that all substituted residues are solvent-exposed and thus constitute areas where mutations are likely to be structurally tolerated. Relative heparin binding affinities were diversely affected in all mutant proteins, as demonstrated by their ability to bind to a heparin-sepharose column and the sodium chloride concentration required for elution. The degree of binding impairment appeared to be related to the number of substitutions and therefore, the decrease of the total electropositive charge.

Mutant R13A/R14A displayed consistent heparin binding characteristics that were reflected in a marked decrease in heparin binding affinity in both analytical affinity chromatography and the filter trapping assay. When Arg14 was targeted in combination with Arg49, the resulting mutant R14A/R49A bound to the heparin column and eluted at the same salt concentration (0.52 M NaCl). Unlike R13A/R14A however, the binding of R14A/R49A to soluble heparin at a salt concentration of 0.15 M was reduced to an extent that Kd values were not measurable (K_{d} values >10 μM).

The triple mutant R13A/R14A/R49A failed to bind heparin at physiological salt concentration in two independent in vitro heparin binding assays. The binding of this mutant to the heparin column at low salt concentration (0.1 M NaCl) and its elution at 0.52 M may be explained by low-affinity electrostatic interactions with the highly concentrated heparin-sepharose at low ionic strength. Binding of the protein at higher ionic strength is prevented due to the saturation, or shielding, of these sites. Taken together, these data indicate that the strong contribution to the binding of heparin comes from a three-arginine cluster and suggest the existence of a principal heparin binding site consisting of Arg13, Arg14, Arg49 and Arg46 with Arg14 and Arg49 representing a minimal binding site. These key heparin binding residues lie along the interface of the clearly defined amino-terminal and carboxy-terminal subdomain and are non-contiguous in sequence. Support for this model comes from the recently published refined NMR solution structure and a much improved definition of the mutual orientation of the amino-terminal and carboxy-terminal subdomain. In contrast to the original structure, the refinement revealed that the heparin-binding residues of the two subdomains (Arg13, Arg14, Arg49) are in close contact with each other, thereby forming a continuous binding surface (Stauffer, M. E. et al. J Biomol NMR, 2002. 23(1): p. 57-61). The mutated residues Arg13, Arg14 and Arg49, although located at opposite ends of the HBD primary sequence, form a continuous binding surface in the tertiary structure.

The effect of the VEGF164 heparin-binding domain mutations on neuropilin-1 binding was examined by determining the IC₅₀ for all VEGF164 mutants in a competitive binding assay in the absence of heparin. The half-maximal concentration of wildtype VEGF164 necessary to inhibit the binding of radiolabeled VEGF165 to rat neuropilin-1 (0.128 nM) is indicative of a high-affinity interaction. This was not expected considering that VEGF is thought to require heparin for efficient binding to neuropilin-1 (Soker, S., et al., J Biol Chem, 1996. 271(10): p. 5761-7). The low IC₅₀ was also in stark contrast with data derived from binding studies using surface plasmon resonance technology, in which the K_{d} of VEGF165 binding to mouse neuropilin-1 was calculated to be 113 nM (Fuh, G. et al. J Biol Chem, 2000. 275(35): p. 26690-5). The latter discrepancy may simply be explained by a lower affinity of mouse and rat neuropilin-1 for human VEGF compared to their human counterpart, although this has not been confirmed. In light of these data, the IC₅₀ values obtained by this approach should be regarded as relative, rather than absolute values. The binding affinity of all mutant VEGF proteins to immobilized neuropilin-1 was reduced between 2.5-fold (K26A) and 115-fold (R13A/R14A/R46A/R49A) compared to VEGF164. However, all mutants retained significant binding activity, suggesting that the neuropilin-1 and the heparin-binding epitopes on VEGF164 are not identical. The residual neuropilin-1-binding activity of the heparin-binding deficient VEGF mutants may account for their ability to induce tissue factor gene expression more potently than VEGF120.

### Example 2

Heparin/VEGF Protein Filter Binding Assay

Purpose:

To determine the binding specificity of heparin towards VEGF164 and mutant VEGF164 variants.

Reagents:

VEGF164 and VEGF164 heparin-binding domain mutant variants (produced by using the Pichia recombinant protein production system from Invitrogen Inc., at Eyetech Research Center, Lexington, Mass.)
[3H]-heparin (Cat # NET476, Perkin Elmer, Inc.)
Scintillation Fluid (Perkin Elmer, Inc.)
   TRIS base, sodium chloride (NaCl) and bovine serum albumin (BSA) (Sigma, Inc.)
      Materials:
Non-Stick 1.5 mL Microfuge Tube (Ambion, Inc.)
Hybridization Oven (Thermo Hybaid, Inc.)
Microbeta TriLux Scintillation Counter (Perkin Elmer, Inc.)
   Millipore Vacuum Manifold and HATF nitrocellular Filter (2.5 cm diameter, 0.45 micron pore size) (Millipore, Inc., Cat# HATF02500)
Pipetman P20, P200, and P1000 (Rainin Instrument Co, Inc.)
Pipet-Aid (Drummond Scientific Co., Inc.)
Sterile Pipets (5 mL, 10 mL, and 25 mL) (VWR Scientific, Inc.)
   Nitrocellulose Binding Assay

Binding of heparin to VEGF was assayed in solution. Serial dilutions of Vascular Endothelial Growth Factor (VEGF164) variants were prepared in binding buffer (25 mM Tris, 150 mM NaCl, 0.1% BSA, pH 7.5) ranging from 5μM to 0.488 nM and were incubated with 0.05 nM of ³H-labeled heparin in microfuge tubes in a final volume of 100 μL (1 h, 37° C.). Solutions (100 μL) were transferred onto nitrocellulose filters (0.45 μM pore size) and protein bound ³H-heparin was then trapped by vacuum filtration using a vacuum manifold. Filters were rinsed three times with 1 mL of washing buffer (25 mM Tris, 150 mM NaCl, pH 7.5) before being transferred to Whatman paper for drying. Protein/heparin complexes were quantified by scintillation counting in a Micro-Beta Scintillation counter.

Saturation binding curves and binding affinities (Kd) were calculated by nonlinear regression analysis (one site binding) using the GraphPad Prism Version 4.0 program.

Analytical Heparin Affinity Chromatography

To determine the heparin binding affinity of VEGF variants, 200 μl of heparin binding buffer (20 mM Tris, 100 mM sodium chloride, pH 7.4) containing 50 μg of protein were loaded onto a preequilibrated 1 ml HiTrap Heparin HP column (Amersham Biosciences) at a flow rate of 0.25 ml/min using the AKTA FPLC™ system (Amersham Biosciences). Unbound material was removed by washing with 1 column volume binding buffer. Proteins were then eluted by a linear salt gradient from 100 mM to 1 M sodium chloride over 9 column volumes at a flow rate of 0.5 ml/min with 0.5 ml fractions collected. The column was reconstituted by washing with binding buffer, then stored in 20% ethanol. Conductivity, pH and UV absorbance (280 nm) was measured at 4° C. Salt concentration for elution of each protein was calculated on the basis of the conductivity of the collected fractions. All fractions were subjected to Trichloroacetic acid precipitation. Dried pellets were diluted in SDS sample buffer, boiled, separated on a 12% SDS-PAGE gel and analysed by Coomassie staining. Method programming as well as analysis and evaluation of runs were done using the Unicorn 4.1 Software (Amersham Biosciences).

Heparin/VEGF Filter Binding Assay

A set of 6 four-fold serial dilutions of the VEGF protein (tube #1 to #6) ranging from 5 μM to 4.88 nM are each mixed with 0.05 μM of ³H-labeled heparin in binding buffer (25 mM Tris, 150 mM NaCl, 0.1% BSA, PH 7.5) in non-stick 1.5 mL microfuge tubes, in a total 100 μL final volume each. Another tube (#7) containing only 0.05 μM of ³H-labeled heparin in 100 μL of binding buffer is used as a background control for the set. The binding reaction is incubated at 37° C. for 1 hr to allow equilibrium binding to occur. Seven HATF nitrocellulose filters are rinsed with wash buffer (25 mM Tris, 150 mM NaCl, PH 7.5) and placed on a Millipore vacuum manifold and pre-wetted with 5 mL of wash buffer under low vacuum (2.5 inches of Hg). While keeping the washed filters under low vacuum, the entire 100 μL of each binding reaction and background control is applied onto the corresponding individual filter and allow to passage through. The filters are immediately rinsed with 1 mL of wash buffer for three times under the same low vacuum. The filters are removed from the manifold, blotted dry briefly on filter paper and transferred to individual scintillation vial. About 3 mL of scintillation fluid is added to each vial, and the radioactivity of each filter is determined by scintillation counting.

Determining Binding Affinities

The amounts of binding in count per minute (cpm) are calculated as: number of counts retained on the filter (#1 to #6) minus the background (filter #7). The resulting corrected binding values in cpm from each target protein (VEGF164 and the variants) dilution and the corresponding target protein concentrations are analyzed by using nonlinear regression with the GraphPad PRISM program (one site binding), and the resulting curve is used to determine the binding affinities (KD) of the heparin towards VEGF164 and the different VEGF164 mutant variants.

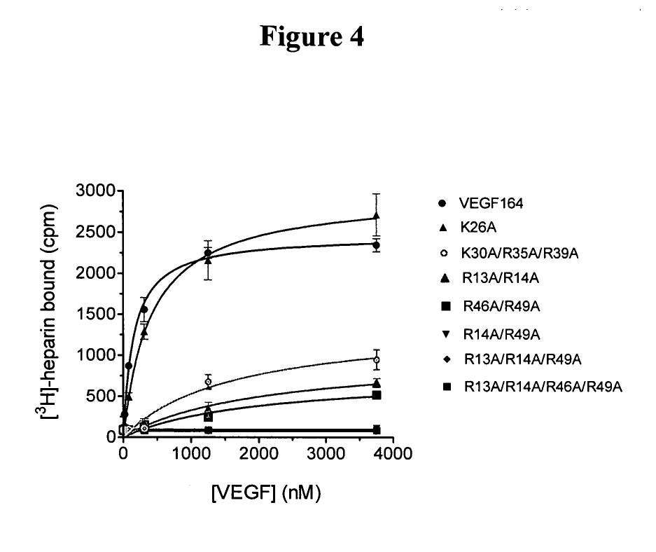

Heparin-binding affinities of VEGF164 (wild type, WT) and the VEGF164 mutant variants were analyzed based on this direct heparin binding assay. The results are illustrated in FIG. 4.

FIG. 4 is a graph showing the heparin-binding affinities of VEGF164 (wild type, WT) and the VEGF164 mutant variants based on a direct heparin binding assay. The amounts of bound ³H-labeled heparin was measured by scintillation counting and is expressed as counts per minute (CPM, Y-axis), and the corresponding concentration of the VEGF protein is expressed in nanomoles (nM, X-axis). The results illustrate that the WT VEGF164 exhibits high affinity, specific and saturation binding for heparin, whereas mutants R14/R49A and R13/R14/R49A exhibit no specific binding toward heparin. Therefore, the results suggest that basic amino acid residues R13, R14 and R49 are important for the heparin-binding activity of VEGF164 heparin-binding domain.

### Example 3

In Vitro Receptor Binding Assays (Competition Binding Assays) to assess VEGF binding to Neuropilin-1, VEGFR1 (Fit-1), and VEGFR2 (Flk-1)

Purpose:

To determine the efficacy of VEGF164 and VEGF164 mutant variants (IC50) in inhibiting the binding of ¹²⁵I-VEGF165 to the three high-affinity cell surface receptors: VEGFR-1, VEGFR-2, and neuropilin-1 in vitro.
   Reagents:
Anti-Human IgG, Fc Fragment-Specific Antibody (CALBIOCHEM, Inc.) Human VEGFR-1/Fc Chimera, Human VEGFR-2/Fc Chimera, Human Neuropilin-1/Fc (R&D Systems, Inc.)
   Bovine Serum Albumin (BSA) and Tween 20 (Sigma, Inc.)
Phosphate Buffered Saline (PBS) (Gibco Life Sciences, Inc.)
Super Block Blocking Buffer in PBS (PIERCE, Inc.)
¹²⁵VEGF165 (Amersham Biosciences, Inc.)
   Materials:
Isoplate High-Binding (HB) 96-well (Cat# 1450-518, Perkin Elmer, Inc.)
Non-Stick 1.5 mL Microfuge Tube (Ambion, Inc.)
Hybridization Oven (Thermo Hybaid, Inc.)
Microbeta TriLux Scintillation Counter (Perkin Elmer, Inc.)
Repeater Plus Pipettor and 10 mL Combitips (Brinkmann Instruments, Inc.)
Pipetman P20, P200, and P1000 (Rainin Instrument Co, Inc.)
Pipet-Aid (Drummond Scientific Co., Inc.)
Sterile Pipets (5 mL, 10 mL, and 25 mL) (VWR Scientific, Inc.)
   Procedures:
   Immobilization of Receptors

For Neuropilin-1, VEGFR-1, and VEGFR-2 binding, 96-well Isoplate plates were first coated with 500 ng (3.33 pmol), 250 ng (1.67 pmol) and 500 ng (3.33 pmol), respectively of anti-human IgG₁ F_{c} fragment-specific antibody in 100μl of PBS (138 mM NaCl, 2.7 mM KCl, 1.5 mM KH₂PO₄, 8.1 mM Na₂HPO₄, pH 7.4) overnight at 4° C. Non specific binding sites were blocked by washing the plates three times with 300 μl of Super Block blocking buffer at room temperature for 5 minutes each. Remaining blocking buffer was washed away with 300 μl of binding buffer (PBS, 0.02% Tween-20, 0.1% BSA, pH 7.4). Subsequently, 0.35 pmol (84 ng) of rat Neuropilin-1/Fc, 0.04 pmol (8.8 ng) of mouse VEGFR-1I/Fc, and 0.2 pmol (44 ng) of mouse VEGFR-2/F_{c} chimeric receptor in 100 μl of binding buffer were immobilized to the corresponding plates for 2 hours at room temperature. Wells were washed two times with 300 μl of binding buffer to remove unbound receptors.

Preparation of the Binding Mix and Competition Binding Assay

A series of five-fold serial dilutions of VEGF164 and VEGF164 variants ranging from 400 nM to 0.02 pM for Neuropilin-1 and VEGFR-2 binding, and from 300 nM to 0.03 pM for VEGFR-1 binding were prepared in binding buffer, and mixed with 0.02 μCi of ¹²⁵I-VEGF165 in microfuge tubes in a final volume of 100 μl. Excess amount of cold VEGF164 (400 nM for Neuropilin-1 and VEGFR-2, and 300 nM for VEGFR-1) was used as background control to determine non-specific binding of ¹²⁵I-VEGF, and maximal binding was determined in the absence of any competitor. The binding samples were transferred to the corresponding wells of the 96-well plate and binding to immobilized receptors was allowed to reach equilibrium (2 hours at room temperature for Neuropilin-1, 2 hours at 37° C. for VEGFR-1 and VEGFR-2). The plate was washed 3 times with a total volume of 900 μl of washing buffer (PBS, 0.02% Tween-20, pH 7.4), before 200 μL of scintillation fluid was added to each well and binding of ¹²⁵I-VEGF was quantified by using a liquid scintillation counter.

Determining the Effective Concentration for 50% Inhibition of Receptor Binding (IC₅₀ Value)

CPM values from each of the three independent experiments were averaged and specific binding of ¹²⁵I-VEGF to receptor was calculated as follows:

$$Specific binding ( \% ) = \frac{X - NSB}{100 \% B - NSB} ⨯ 100$$

X=mean CPM value specific for each concentration of cold competitor
NSB=mean CPM value for non-specific binding (presence of excess amount of cold competitor)
100%B=maximal binding of ¹²⁵I-VEGF in the absence of cold competitor

Competition binding curves and IC₅₀ values for VEGF164 and the different VEGF164 mutant variants were calculated by nonlinear regression analysis (one site competition) using the GraphPad Prism Version 4.0 program.

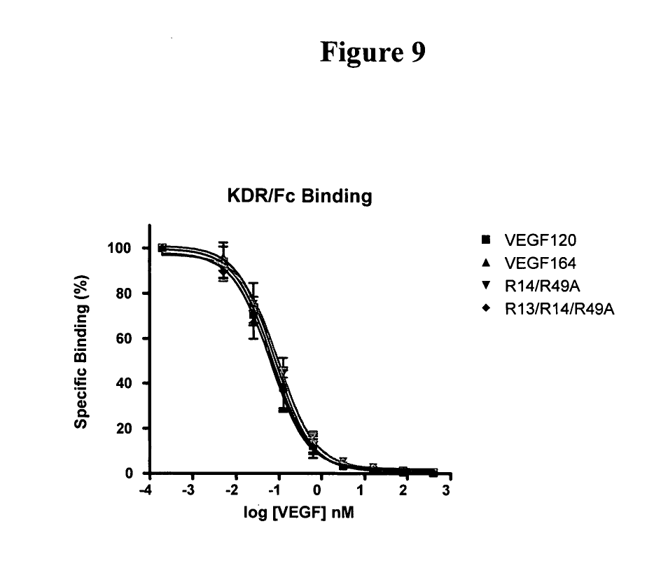

VEGF binding to Neuropilin-1 (Np-1), VEGFR1 (Flt-1), and VEGFR2 (Flk-1) was analyzed using in vitro receptor binding assays and the results of these in vitro receptor binding assays are illustrated in FIGS. 9 through 12.

FIG. 9 is a graph showing the results of an in vitro VEGF/VEGF-receptor-2 (KDR) competition plate binding assay for VEGF isoforms and the VEGF164 mutant variants. Increasing amounts of the different cold competitors (VEGF120, VEGF164, mutant R14/R49A, and mutant R13/R14/R49A) (X-axis) were used to compete with ¹²⁵I-labeled VEGF165 for the binding with KDR receptor. The levels of specific binding by the ¹²⁵I-labeled VEGF165 at increasing concentrations of the cold competitors are expressed as percentage binding on the Y-axis. The graph illustrates comparable potencies in inhibiting VEGF165/KDR receptor binding by VEGF120, VEGF164 and the VEGF164 heparin-binding domain mutant variants (R14/R49A and R13/R14/R49A). Therefore, both wild type VEGF164 and mutants variants have similar binding affinity toward the KDR receptor. The results confirm that the mutagenesis in the heparin-binding domain residues R13, R14 and R49 does not affect the KDR receptor binding site of VEGF164.

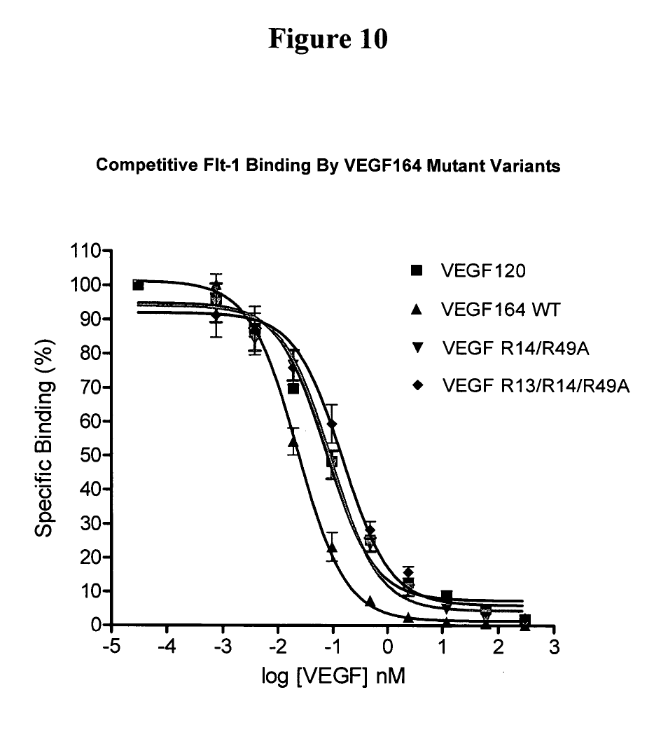

FIG. 10 is a graph showing the results of an in vitro VEGF/VEGF-receptor-1 (Flt-1) competition plate binding assay for VEGF isoforms and the VEGF164 mutant variants. Increasing amounts of the different cold competitors (VEGF120, VEGF164, mutant R14/R49A, and mutant R13/R14/R49A) (X-axis) were used to compete with ¹²⁵I-labeled VEGF165 for the binding with Flt-1 receptor. The levels of specific binding by the ¹²⁵I-labeled VEGF165 at increasing concentrations of the cold competitors are expressed as percentage binding on the Y-axis. The graph illustrates decreased potency in inhibiting VEGF165/Flt-1 binding, and therefore decreased Flt-1 receptor binding affinities by VEGF164 heparin-binding domain mutant variants (R14/R49A and R13/R14/R49A) compared to the wild-type (WT) VEGF164. Furthermore, VEGF120, which lacks the heparin-binding domain, also exhibited lower potency in inhibiting VEGF₁₆₅/Flt-1 binding compare to the WT VEGF164. The results suggest that the heparin-binding domain and specifically the residues R13, R14 and R49 of the heparin binding domain are important for the high affinity binding of Flt-1 receptor by VEGF164.

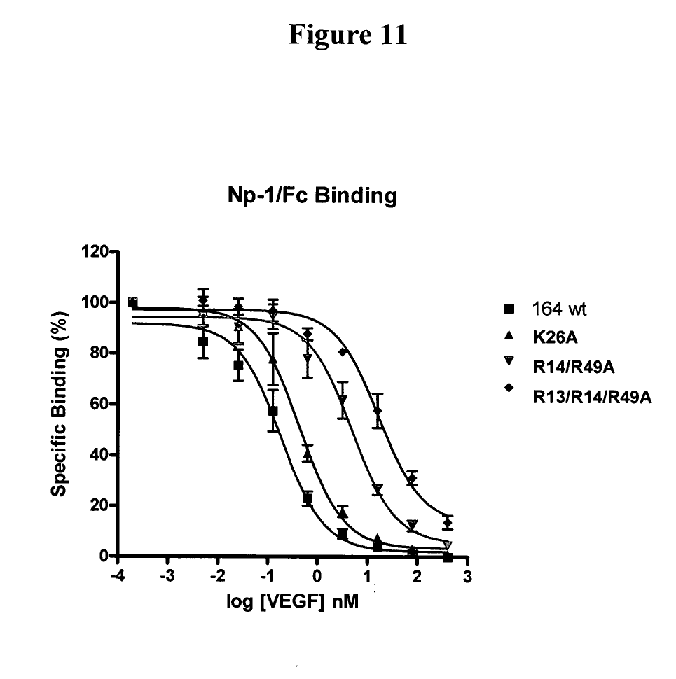

FIG. 11 is a graph showing the results of an in vitro VEGF/neuropilin-1 (Np-1) receptor competition plate binding assay for wild-type (WT) VEGF164 and the different mutant variants (mutants K26A, R14/R49A, and R13/R14/R49A). Increasing amounts of the different cold competitors (VEGF164, mutants K26A, R14/R49A, and R13/R14/R49A) (X-axis) were used to compete with 125I-labeled VEGF165 for the binding with Np-1 receptor. The levels of specific binding by the ¹²⁵I-labeled VEGF165 at increasing concentrations of the cold competitors are expressed as percentage binding on the Y-axis. The graph illustrates decreased potencies in inhibiting VEGF165/Np-1 binding, and therefore decreased binding affinities to Np-1 receptor by all the VEGF164 heparin-binding domain mutant variants K26A, R14/R49A, and R13/R14/R49A when compared to the WT VEGF164. Furthermore, because mutant K26A has retained some of the heparin-binding activity that is higher than either mutant R14/R49A and R13/R14/R49A, the heparin-binding activities of the mutant variants exhibit a positive correlation with their binding affinities toward Np-1. The results suggest that the heparin-binding domain and especially residues R13, R14, and R49 are involved in the high affinity binding of Np-1 receptor by VEGF164, and that the heparin-binding activity has a positive correlation with the binding affinity of VEGF164 for Np-1 receptor. The results also suggest that the binding sites for heparin and Np-1 partially overlap.

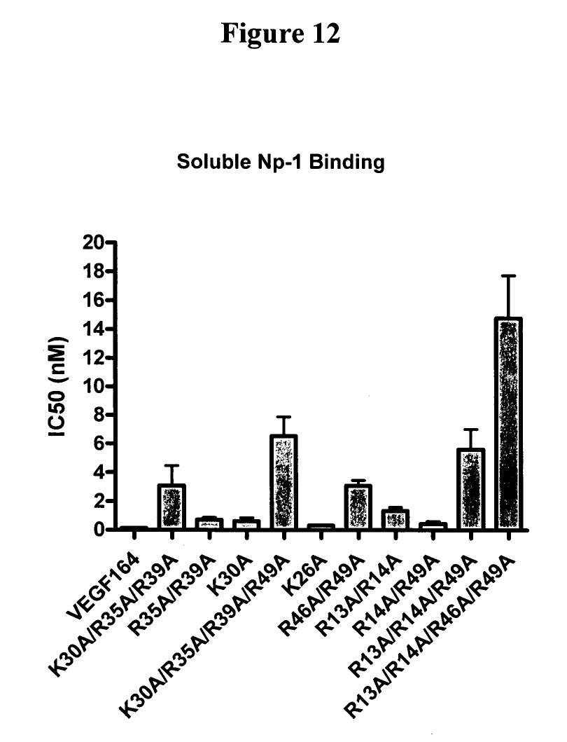

FIG. 12 is a chart showing the quantified results of the in vitro VEGF/neuropilin-1 (Np-1) receptor competition plate binding assay for wild-type (WT) VEGF164 and the different mutant variants (mutants K26A, R14/R49A, and R13/R14/R49A). The potency of inhibiting VEGF165/Np-1 binding by VEGF164 and the variants are expressed as IC50 on the Y-axis. The chart shows decreased potencies of inhibiting VEGF165/Np-1 binding (increased IC50 values) by the VEGF164 heparin-binding domain mutant variants K26A, R14/R49A, and R13/R14/R49A when compared to the wild type VEGF164. Also, the mutant that retained most of the heparin-binding activity (mutant K26A) also exhibited the least decrease in potency in inhibiting VEGF165/Np-1 binding (lowest IC50 among the variants), suggesting a positive correlation between heparin-binding activity and affinity for Np-1 binding by VEGF164.

### Example 4

Intravitreous Injection of VEGF and Acridine Orange Reukocyte Fluorography

VEGF heparin-binding domain is responsible for this enhanced effect, recombinant VEGF164 mutants were tested for their potency in inducing leukocyte adhesion to the retinal endothelium.

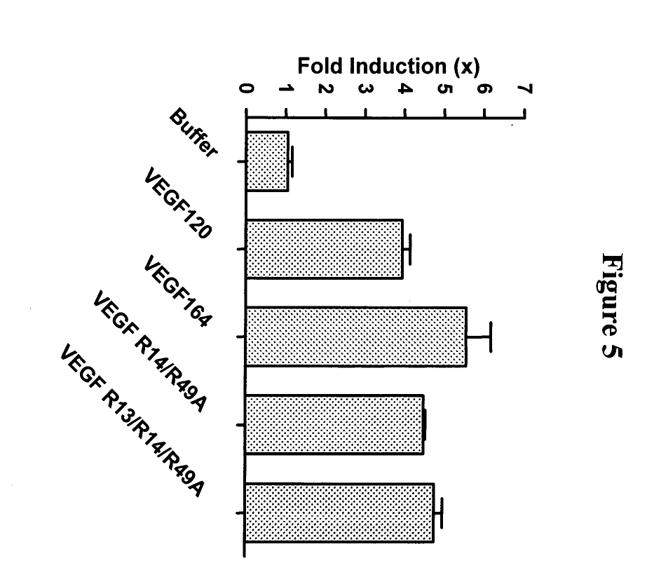

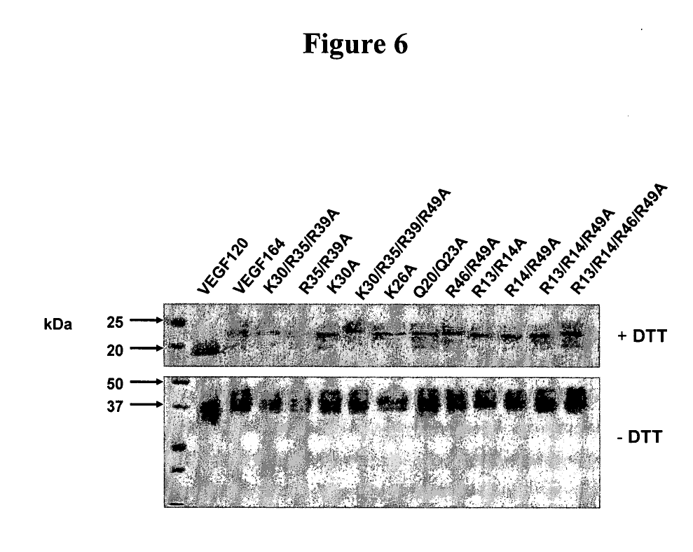

Equimolar concentrations of purified and sterilized VEGF variants were administered intravitreally into rats and leukocyte accumulation in the retina was analyzed after one, two or three days by in vivo leukocyte fluorography. As shown in FIG. 5.6A, VEGF164-induced leukostasis in the retinal microvasculature peaked at 48 hours after injection (52.6±8.3 leukocytes/mm² retinal surface area) and was approximately 3-fold higher than the leukostasis induced by VEGF120 (17.7±2.7 leukocytes/mm²). When compared with the VEGF164 control, these findings indicate that the leukocyte recruitment was specific, and directly caused by active VEGF. These data are comparable with results from previous studies in which VEGF164 induced a 1.9-fold greater increase in leukostasis than did VEGF120.

A single 2 pmol or 20 pmol intravitreous injection of inactivated VEGF164, VEGF164, VEGF120, and various VEGF164 mutant variants in 5 μL PBS was performed by inserting a 33-gauge needle into the vitreous of anesthetized rats. The dosage was determined based on a previous report describing leukostasis in the retinal vasculature after intravitreous injections of VEGF165. Male Long Evans rats, weighing 200-225 g, were used in this experiment. Insertion and infusion were performed under surgical microscope observing retinas directly. At 24, 48, and 72 hours after vitreous injection, Leukocyte dynamics in the retina were studied with acridine orange digital fluorography (AODF). The optic media (which consists of cornea, lens, vitreous, and retina) are so transparent that the retinal microcirculation could be observed noninvasively by employing AODF. Intravenous injection of acridine orange causes leukocytes and endothelial cells to fluoresce through the noncovalent binding of the molecule to double-stranded nucleic acid. When a scanning laser ophthalmoscope (SLO: Rodenstock Instruments, Munich, Germany) is used, retinal leukocytes within blood vessels can be visualized in vivo. Each leukocyte was recognized as a single fluorescent dot moving in the retinal vessels. It was possible to analyze the spatial and temporal dynamics of individual leukocytes in the capillaries. In physiological condition, some leukocytes passed through the capillaries plugging transiently. Leukocytes that stayed in the same position for a few minutes may have stuck to the endothelium as a result of leukocyte-endothelial interactions. At 30 min after injection, acridine orange injected into the body being washed out, static leukocytes in the capillary bed, if present, can be observed as white still dots.

At each time point and immediately before AODF, each rat was again anesthetized, and the pupil was dilated with 1% tropicamide to observe leukocyte dynamics. Acridine orange (Sigma, Inc.) was dissolved in sterile saline (1.0 mg/mL) and 3. mg/kg was injected through the tail vein catheter at a rate of 1 mL/minute. The fundus was observed with the SLO using the argon blue laser as the illumination source and the standard fluorescein angiography filter in the 40 degree field setting for 1 minute. Thirty minutes later, the fundus was again observed to evaluate retinal leukostasis. The images were recorded on a digital videotape at the rate of 30 frames per second. The recorded images were analyzed on a computer into which the video images were taken in real time (30 frames per second) to 640×480 pixels with an intensity resolution of 256 steps. For evaluating retinal leukostasis, an observation area around the optic disk measuring five disk diameters in radius was outlined by drawing a polygon bordered by the adjacent major retinal vessels. The area was measured in pixels and the density of trapped leukocytes was calculated by dividing the number of static leukocytes, which were recognized as fluorescent dots, by the area of the observation region. A leukocyte was considered static if its position did not change for 3 minutes. The density of leukocytes was calculated in 8 peripapillary observation areas and an average density was obtained by averaging the 8 density values.

Statistical Analysis

All values were expressed as mean±SE. The unpaired Student t test was used for statistical analysis when compared two groups and the data were analyzed by using post hoc comparisons test when compared three or more groups. Differences were considered statistically significant when the P values were <0.05.

Intravitreous Injection of VEGF and Acridine Orange Leukocyte Fluorography

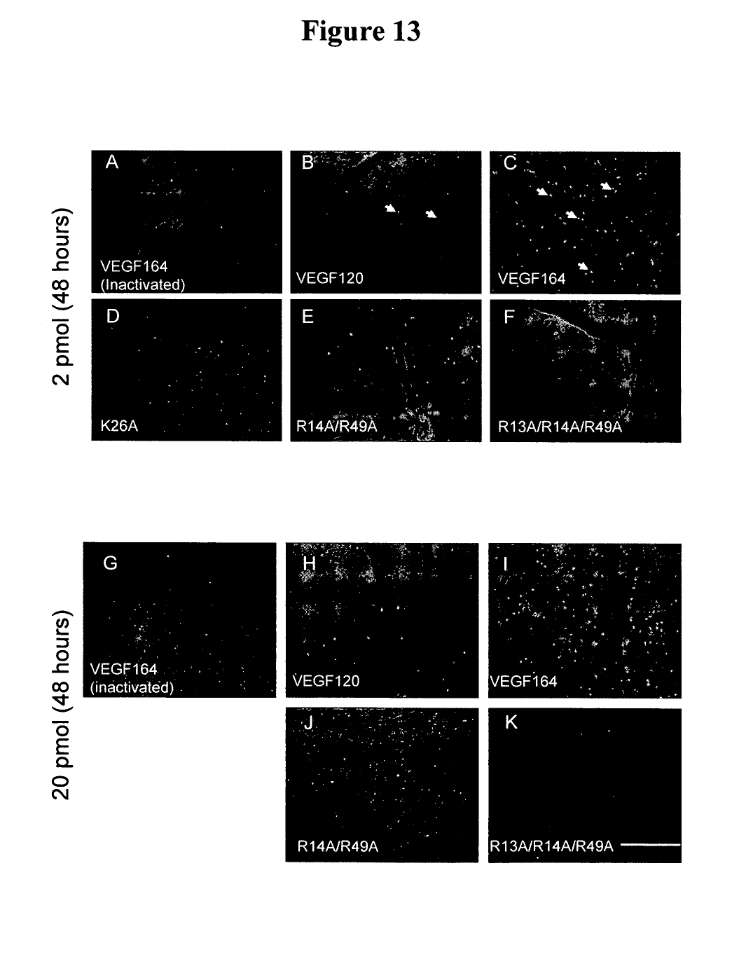

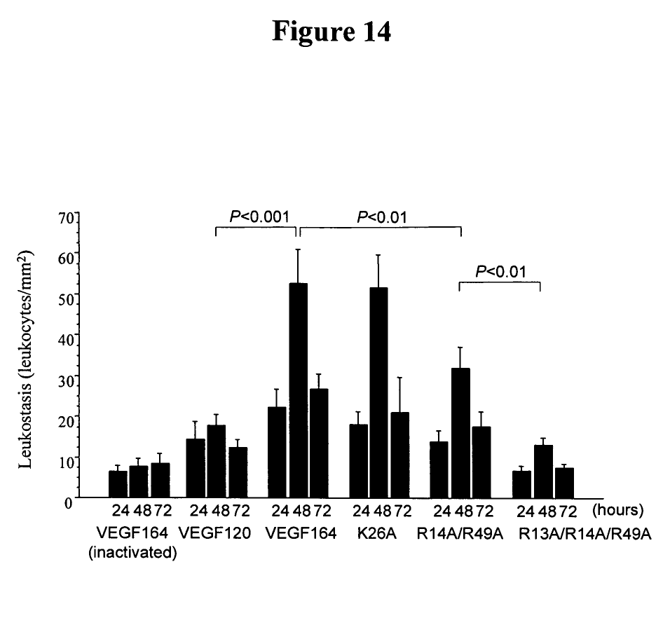

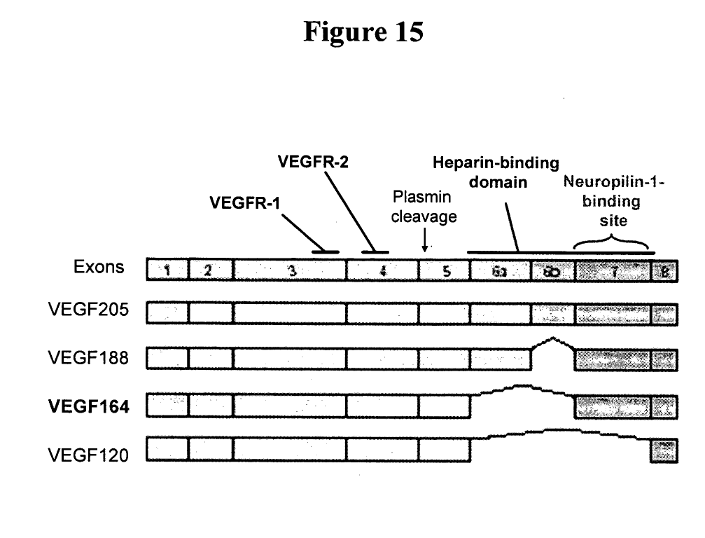

The activity of VEGF its isoforms and variants to recruit leukocytes were analyzed using the above-described acridine orange leukocyte fluorography. The results of the effect of VEGF variants on leukostasis are shown in FIGS. 13 and 14.

FIG. 13 shows Scanning Laser Ophthalmascope (SLO) images of rat retinas post injection with VEGF to induce leukostasis and acridine orange. Five images are shown in FIG. 13 including those of VEGF164, Inactivated VEGF164, VEGF120, Mutant R14/R49A and Mutant R13/R14/R49A. The light dots on the images are leukocytes. Wild type VEGF164 shows numerous dots while Mutant R14/R49A and Mutant R13/R14/R49A show far less. The images illustrate that the heparin-binding domain mutants of VEGF164 have much reduced activities to induce leukostasis in the retina.

FIG. 14 is a chart showing the quantified results of the modulation of leukostasis by VEGF164 and its variants. The vertical axis represents leukostasis measured by the density of leukocytes in terms of area measured in pixels from an SLO image. SLO images of each VEGF isoform and variant were measured at 24, 48 and 72 hours. The chart illustrates that the heparin-binding domain mutants are significantly less potent in inducing leukostasis in the retina.

FIG. 13 and 14 illustrate leukocyte recruitment to the rat retinal vasculature after intravitreal injection of VEGF wild-type and mutant variants. (A) Time course of leukocyte dynamics after intravitreal injection of 2 pmol of purified Pichia-derived protein. The dosage was determined based on a previous report describing leukostasis in the retina after VEGF injection (Miyamoto, K., et al.,. Am J Pathol, (2000). 156(5): p. 1733-9). VEGF164 was inactivated by boiling for 10 minutes and served as a control. Leukocytes were labeled by injecting acridine orange intravenously 30 minutes before scanning laser ophthalmoscopy (SLO). To evaluate retinal leukostasis, the number of fluorescent dots within 8 areas (each 2002 pixels²) at a distance of 5 disc diameters from the edge of the optic disc was counted. These numbers were converted to leukocytes/mm² by using the formula: 1 pixel2=3.22 μm². At least 6 eyes were counted per time point and protein (N≧6). (B-L) Representative acridine orange leukocyte fluorography (AOLF) images of the eye fundus 48 hours after intravitreal injection of 2 pmol (B-F) and 20 pmol (G-K) VEGF. Adherent leukocytes appear as white dots (arrows). No increase in leukostasis was observed in 20 pmol vs. 2 pmol injected eyes. Scale bar (K): 500 μm.

In contrast, the VEGF164 mutants R14A/R49A and R13A/R14A/R49A were increasingly less effective at inducing leukocyte recruitment to the retinal capillary bed compared with VEGF164. Only 31.9±5.1 leukocytes/mm2 and 13.1±1.6 leukocytes/mm were counted 48 hours after injection of the double mutant and the triple mutant, respectively (FIGS. 13, E and F).

Applicants studied whether the reduced potency of the two mutants with respect to leukostasis was specifically associated with alterations in the region implicated in heparin binding. The single mutant K26A was therefore used as a control, since binding studies indicated that lysine 26 is not part of the heparin-binding site. K26A retained wild-type potency 48 hours after injection of 2 pmol (51±7.9 leukocytes/mm²) suggesting that arginine 13, 14 and 49 constitute residues that are important for mediating the pro-inflammatory activity of VEGF164. The reduced ability of the mutants to induce leukostasis did not result from low dosage as qualitative analysis of the eye fundus did not show an increase in leukocyte infiltration after injecting 20 pmol of protein when compared with 2 pmol (FIGS. 13J and K).

To confirm the identity of the infiltrating blood cells in the retinal vasculature, weight-matched mice were perfused with FITC-labeled concanavalin A lectin (ConA) 24 hours after intravitreal injection of VEGF164 to image the retinal vasculature and leukocytes.

The results suggest that the heparin-binding domain confers the pro-inflammatory activity of VEGF164 and that modifying the heparin binding domain of VEGF as described herein reduces the ability of VEGF to recruit leukocytes and thereby inflammation.

### Example 5

VEGF-Mediated Tissue Factor induction Assay in HUVEC Cells:

1. Cell Culture and RNA Isolation

HUVEC Cells at passage 3 or lower (Cascade Biologics, cat# C-015-10C) are plated in complete medium ( Medium 200 Cascade Biologics cat# 200-500, supplemented with Low Serum Growth Supplement cat# S-003-10) at a density of 3.0×10⁵ cells per well in 12 well plates. Cells are allowed to attach overnight in a humidified tissue culture incubator at 37° C. and 5% CO₂. The next morning, cells are washed once in minimal medium (Medium 200 supplemented with 1% Fetal Bovine Serum from Gibco, cat# 16000-036) and cultured in this minimal medium for four hours in a humidified tissue culture incubator at 37° C. and 5% CO₂ before treating with VEGF.

Using sterile techniques in a tissue culture hood, tubes are labeled and different samples of minimal culture medium containing 12.5 ng/mL of VEGF164 and the different VEGF164 mutant variants (Produced at Eyetech Research Center, Lexington, Mass.) are prepared. Each experimental condition is done in triplicate (3 wells) using 1 mL of medium per well per treatment. Minimal medium with no VEGF added is used as negative control.

At the end of the four hour incubation in minimal medium, HUVEC cells are treated with the minimal medium containing VEGF for I hour in a humidified tissue culture incubator at 37° C. and 5% CO₂. Cells are then washed with 1 mL of PBS gently without dislodging any cells, and 350 μL of lysis buffer RLT from the RNeasy® kit from Qiagen (cat# 74104) is added to the cells. Cell lysates are collected in clean nuclease-free microfuge tubes and placed immediately on ice and used for RNA isolation according to the manufacture's protocol.

2. Real-Time RT-PCR (TaqMan) Analysis of Tissue Factor Expression

RNA samples isolated from the HUVECs are treated with DNase using the DNA-free kit (Ambion, Cat# 1906) according to manufacturer's protocol to remove any contaminating genomic DNA. 300 ng of the resulting DNA-free RNA is used for cDNA synthesis using the TaqMan Reverse Transcription Reagents (ABI, Cat#N8080234) with both oligo d(T) 16 and random primers in a total of 60 μL volume, and according to manufacturer's protocol. With the resulting cDNA, 2 μL is used for each TaqMan analysis with specific primers and TaqMan probes for tissue factor. In a separate reaction with specific primers and TaqMan probes for GAPDH is used as an internal normalization control. Each cDNA sample is subjected to duplicated TaqMan analysis, and the average of the two results is used for the subsequent calculation. The results of the TaqMan analysis is expressed as fold induction of tissue factor expression compared to the untreated (no VEGF) HUVEC RNA samples.

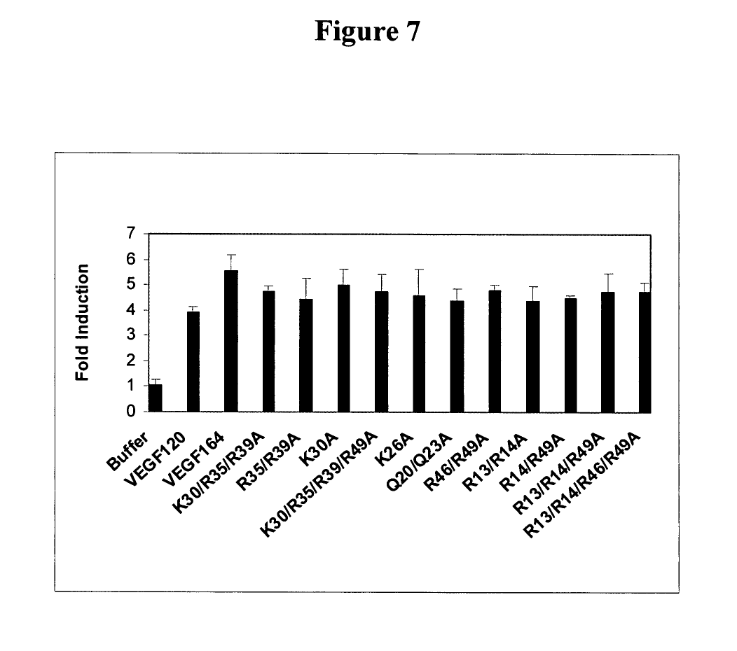

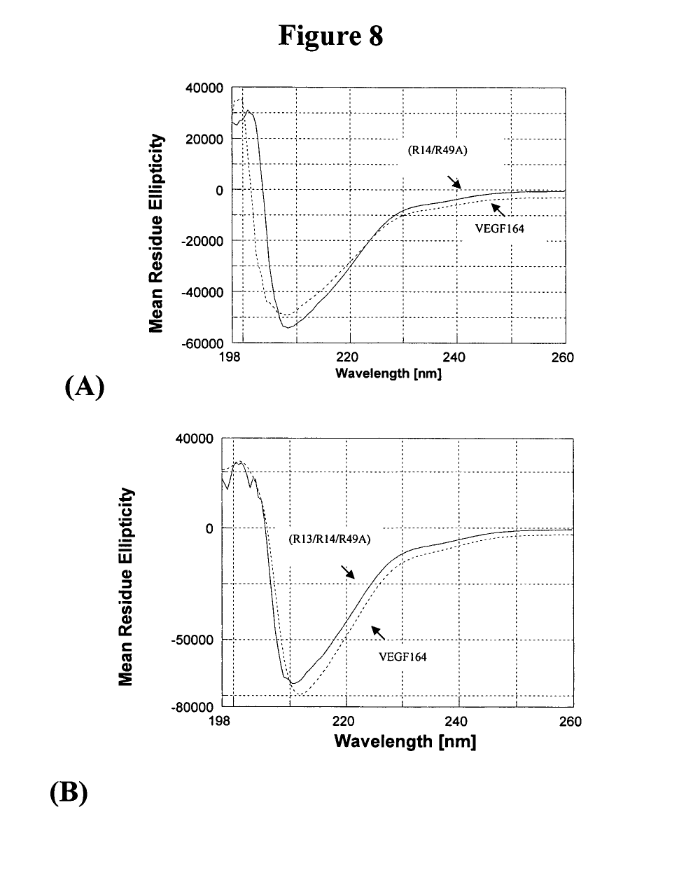

The activity of VEGF and its variants were analyzed by monitoring the induction of tissue factor (TF) gene expression in a cell based assay using HUVECs (Human Umbilical Vein Endothelial Cells). VEGF induces the expression of the TF gene in HUVEC through its high affinity receptors, VEGFR-1 and VEGFR-2. The tissue factor gene is a cellular initiator of the coagulation cascade through binding to Factor VII. The results of the HUVEC Tissue Factor assay are shown in FIG. 7. In FIG. 7, the vertical axis shows the fold induction of tissue factor gene expression in HUVEC resulting from the VEGF isoforms and VEGF variants, which are indicated on the horizontal axis. The fold induction of tissue factor expression correlates with the functionality of the VEGF isoforms and VEGF variants. FIG. 7 illustrates that all VEGF mutants are fully functional and are similar to the wild-type VEGF164 in the HUVEC Tissue Factor Assay. The results suggest that modifying the heparin binding domain as described herein has no effect on a normal VEGF function in inducing TF expression in endothelial cells.

### Example 6

Characterization of VEGF164 Exon 7 Mutants in an In Vitro Model of Angiogenesis

The ability of the VEGF164 mutants to induce angiogenesis in vitro was assessed in a rat aortic ring organ culture model. Rat aorta rings generate microvessel outgrowth and a network composed of branching endothelial tubes. This assay is known to reproduce more accurately the environment in which angiogenesis takes place than other in vitro assays. Furthermore, cultures can be maintained in a defined, serum-free growth medium allowing for the evaluation of exogenous factors.

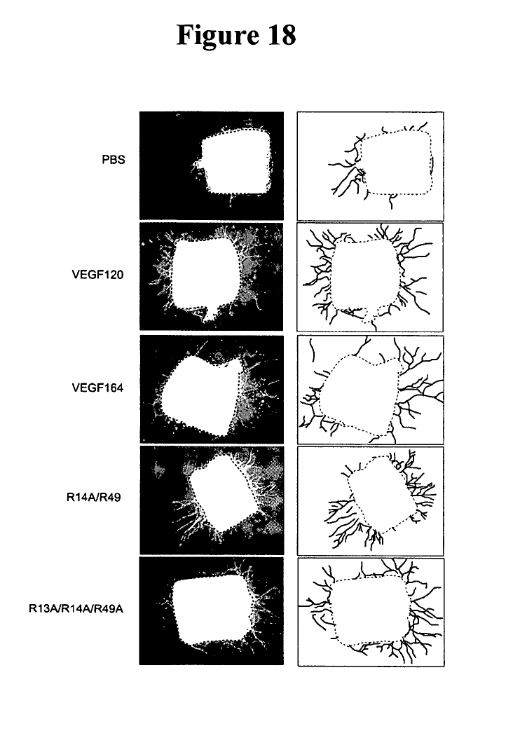

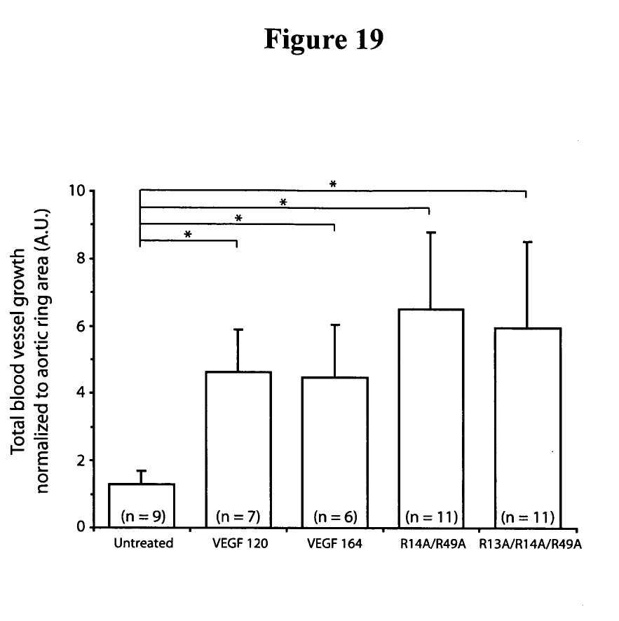

To test the effect of the VEGF exon 7 mutants on angiogenesis in the aortic ring model, segments of the aorta were embedded in collagen and incubated with serum-free medium in the presence or absence of VEGF120, VEGF164, R14A/R49A or R13A/R14A/R49A. FIGS. 18 and 19 illustrate the potency of recombinant VEGF164 exon 7 mutants and wild-type isoforms in a rat aortic ring model After 7 days in culture, rings of each group gave rise to branching microvessels extending mostly from the edge of the ring and were surrounded by elongated fibroblast-like cells (FIG. 18). Isolectin B staining of vessels revealed that PBS-treated control rings produced few well formed vascular sprouts induced by the release of endogenous growth factors (FIG. 18, left panels). The treatment with equimolar concentrations of either VEGF120 or VEGF164 induced an increase in total length of microvessels that was 3.5-fold (for VEGF120) and 4-fold (for VEGF164) higher than background levels (FIG. 19). Similarly, R14A/R49A and R13A/R14A/R49A consistently stimulated a high level of sprouting. Quantification of the total vessel length revealed a 6-fold (for R14A/R49A) and 5.5-fold (for R13A/R14A/R49A) increase compared to PBS-treated rings (FIG. 19). No gross differences in sprout morphology were observed when rings from different groups were compared. These data demonstrate that the mutations introduced into the heparin-binding domain of VEGF164 did not negatively affect the growth of microvessels in the aortic ring assay.

### Example 7

Determination of the Heparin Binding Activity of the Mutants (In Vitro Activity)

Applicants employed a heparin-sepharose chromatography as a screening method for testing the heparin binding affinity of the VEGF mutants. A heparin-sepharose column was loaded with purified protein dimers, washed, and bound proteins were eluted with a linear sodium chloride gradient. The relative affinity for heparin was then assessed by determining the amount of salt required to elute the proteins from the column.

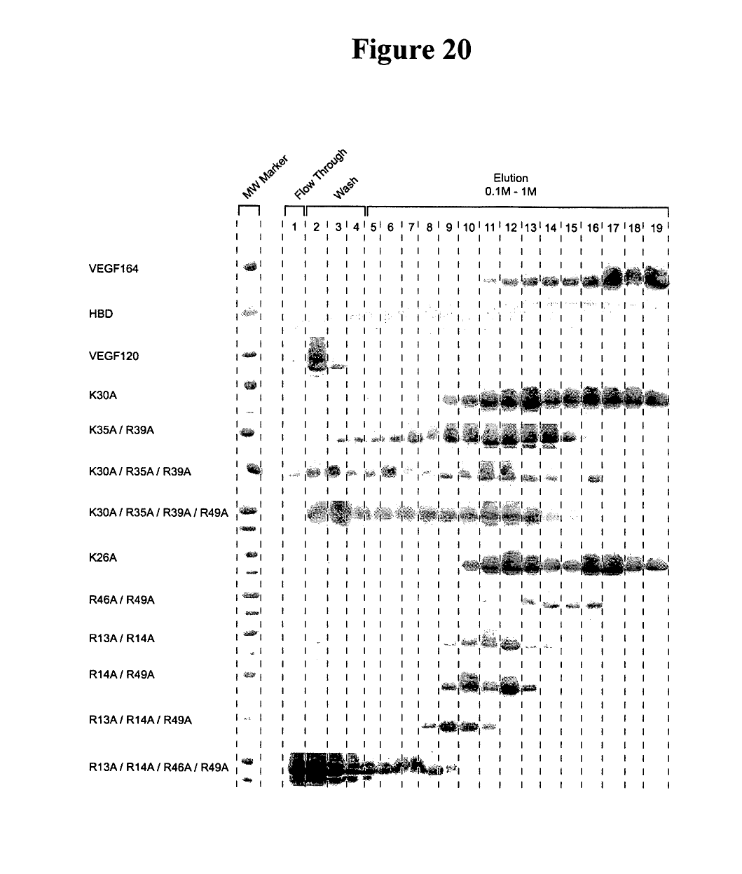

VEGF164 completely bound to the heparin column in the presence of 0. 1 M NaCl (FIG. 20). Binding of VEGF to heparin occurred through binding determinants located in its heparin binding domain, since VEGF120, which lacks this region, did not bind to the column and was found in the flow-through and wash fractions. In addition, VEGF55 displayed similar heparin binding behavior to VEGF164, resulting in a similar elution profile. (These data confirm that all of the heparin binding activity of VEGF164 is mediated by its heparin binding domain). VEGF164 eluted from the column over a wide range of the salt gradient (0.52M -0.94 M NaCl). The concentration of sodium chloride in the elution buffer required to displace 50% of the protein from the column was used as an indicator for heparin binding affinity.

FIG. 20 shows purified protein dimers (10 μg) applied to a heparin-sepharose affinity column in binding buffer containing 0.15 M sodium chloride. The fall-through was collected before the column was washed in binding buffer. Bound proteins were eluted over 10 ml in a linear salt gradient to 1.5 M sodium chloride-tris buffer and 1 ml fractions were collected. Fractions were precipitated with trichloroacetic acid and separated on a 12% SDS-PAGE gel. Western blotting was performed using a monoclonal VEGF antibody and immuno-positive bands were visualized with a chemiluminescence system.

Without wishing to be bound by theory, the basic amino acids Lys 30, Arg 35, Arg 39 and Arg 49 in the carboxy-terminal domain are located in close proximity to each other and thus may potentially act as docking sites for GAG chains. The quadruple mutant K30A/R35A/R39A/R49A and the triple mutant K30A/R35A/R39A presented a similar elution profile. In both cases a significant amount of protein was found in the wash and early elution fractions and a second fraction bound more tightly to the column and eluted at approximately 0.46 M (FIG. 20). This variability suggests that the protein was partly degraded and that mutations in this region may have rendered the protein more susceptible to degradation or misfolding.

The binding of the single mutant K30A was investigated in order to determine the relative contribution of this mutation to the heparin binding behavior observed with the double and triple mutant. No significant difference in the elution characteristics was detected between this mutant and wildtype VEGF164.

Arg46 and Arg49 form a basic cluster that is part of the two-stranded antiparallel β-sheet structure in the carboxy-terminal domain. Targeting of these residues resulted in a slightly decreased binding capacity of the protein as shown in FIG. 20. The NaCl concentration required to displace 50% of this mutant from the heparin column was approximately 0.64 M. Heparin binding was further impaired in the double mutant R13A/R14A (0.52 M NaCl). Arg 13 and Arg14 form the disordered and poorly defined loop region adjacent to Arg 46 and Arg49 and the combination of these two mutants (R13A/R14A/R46A/R49A) resulted in almost complete disruption of heparin binding. Both variants bound to the column and eluted over a relatively narrow range of salt concentration, which was significantly lower than VEGF164 (0.76 M). Double mutant R14A/R49A showed a distinct reduction to 0.52 M and an even greater reduction was observed with the triple mutant R13A/R14A/R49A (0.4 M). These results indicate the presence of a heparin binding site in a region that comprises Arg 13, Arg 14, Arg 46 and Arg 49.

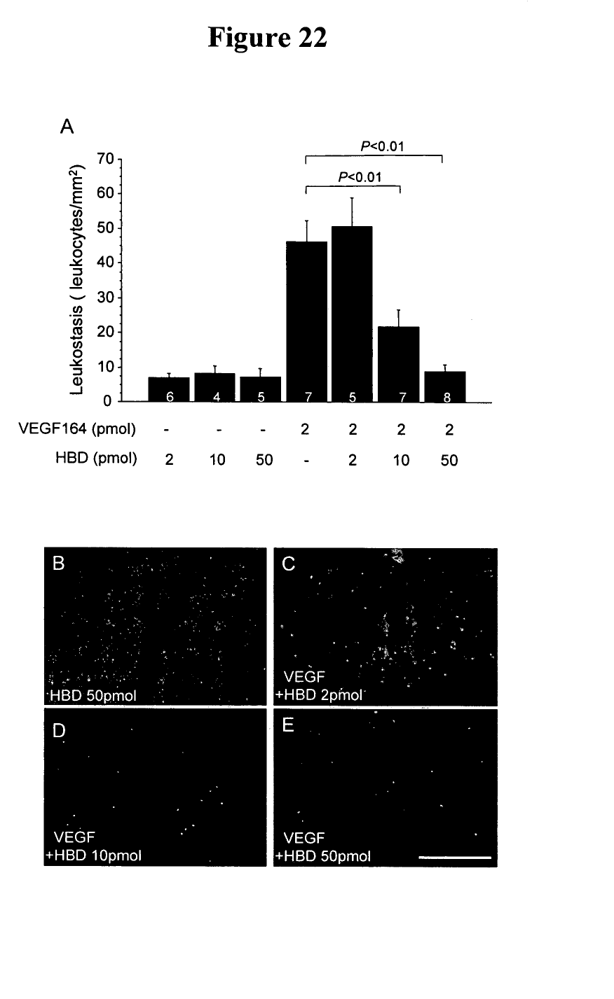

To further investigate these observations, VEGF164 and the mutants R14A/R49A and R13A/R14A/R49A were tested again in the same assay under slightly different experimental conditions, increasing both the salt concentration in the binding buffer (0.15 M NaCl) and the NaCl concentration increment per fraction. Under these conditions, VEGF164 bound to the column and eluted at approximately 0.82 M NaCl (FIG. 22), which is consistent with the previous experiment.

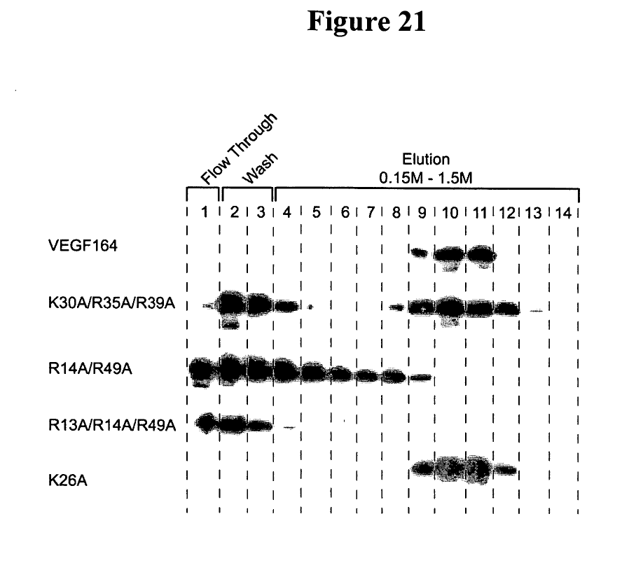

FIG. 21 illustrates the heparin-binding behavior of VEGF164 wildtype and select mutants at physiological salt concentration. Purified protein dimers (10 μg) were applied to a heparin-sepharose affinity column in binding buffer containing 0.15 M sodium chloride. The fall-through was collected before the column was washed in binding buffer. Bound proteins were eluted over 10 ml in a linear salt gradient to 1.5 M sodium chloride-tris buffer and 1 ml fractions were collected. Fractions were precipitated with trichloroacetic acid and separated on a 12% SDS-PAGE gel. Western blotting was performed using a monoclonal VEGF antibody and immuno-positive bands were visualized with a chemiluminescence system.

Mutant R13A/R14A/R49A lost its ability to bind to the heparin-sepharose column at physiological salt concentration, suggesting that the binding activity observed in the previous experiment may have been due to non-specific electrostatic contributions to the interaction. Analysis of the double mutant R14A/R49A revealed that heparin binding was compromised as the majority of protein was found in the flow-through and the wash fractions. A fraction of the protein, however, bound to the column and eluted gradually between 0.15 M and 0.6 M.

### Example 8

Soluble Heparin-Binding Domain (HBD) Inhibits VEGF164-Induced Leukostasis

The VEGF C-terminal domain may either directly or indirectly mediate the pro-inflammatory activity of VEGF164. To examine whether this region by itself can induce this effect, the heparin-binding domain of VEGF164 was expressed in yeast cells as a recombinant fragment (HBD) and injected into rats. As shown in FIG. 22A, intravitreal injection of 2, 10 or 50 pmol of the purified peptide, did not increase leukostasis significantly above control levels (7.6±2.1 leukocytes/mm²). The results suggests that the VEGF heparin-binding domain cannot exert its pro-inflammatory potential independently of the N-terminal receptor-binding domain but only in the context of the full-length protein.

Because soluble HBD lacks the ability to induce leukostasis (does not produce a leukostasis phenotype) observed with VEGF164, it may be able to interfere with VEGF-induced retinal leukostasis. To investigate this possibility, 2, 10 and 50 pmol of recombinant HBD was injected intravitreally 2 minutes before VEGF164 using an injection-delay technique that does not require the removal of the needle between the two injections.

The HBD was found to potently inhibit VEGF-induced leukocyte adhesion to the retinal microvasculature in a dose-dependent manner (FIGS. 22A and C-E). A 25-fold molar excess of the HBD monomer (50 pmol) over the VEGF dimer (2 pmol) resulted in a marked reduction of VEGF-induced leukostasis (8.8±2.13 leukocytes/mm²). This level was comparable to that observed after injecting inactivated VEGF164 (7.6±2.1 leukocytes/mm²). Thus, in one embodiment, the VEGF heparin-binding domain acts an anti-inflammatory agent in vivo by interfering with VEGF164 activity in the eye.

Applicants investigated the mechanism by which soluble HBD interferes with VEGF164-induced leukocyte recruitment. Following intravitreal injection, HBD, like VEGF, diffuses through the vitreous humor and the neuronal cell outer nuclear layer (ONL) layer before it reaches the retinal vessels. One cannot exclude the possibility that HBD associates non-specifically with VEGF in the vitreous, whereby VEGF may be rendered unable to interact with receptors. Without wishing to be bound by theory, Applicants hypothesize that interference occurs through competition between the HBD and VEGF164 for binding to heparan-sulfate proteoglycans and/or neuropilin-1, thereby blocking potential docking sites for VEGF on retinal endothelial cells.

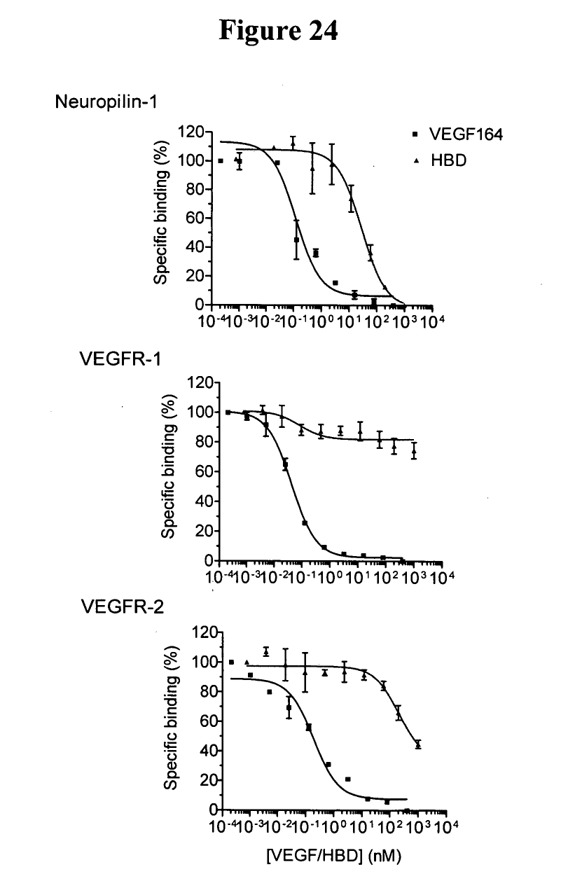

Applicants determined the ability of HBD to compete with ¹²⁵I-VEGF165 for binding to immobilized neuropilin-1, as well as VEGFR-1 and VEGFR-2 in the in vitro competitive binding assay. Neuropilin-1-binding activity of VEGF is conferred by the heparin-binding domain. Indeed, HBD was able to completely displace ¹²⁵I-VEGF165 from immobilized neuropilin-1, resulting in a half-maximal inhibitory concentration (IC₅₀) of 28.56±4.5 nM (FIG. 24, top panel). Competitive binding of dimeric VEGF164 to neuropilin-1 was approximately 230-times stronger.

The HBD did not bind significantly to VEGFR-1, even at concentrations as high as 1 μM (FIG. 24, middle panel). The HBD was able to compete with VEGF for binding to VEGFR-2 at very high concentrations (FIG. 24, bottom panel). The competitive behaviour exhibited by HBD may have been due to non-specific association with the receptor rather than competition for the same binding site. This in vitro analysis showed that recombinant HBD competes with VEGF164 for binding to neuropilin-1, but not to VEGFR-1 or VEGFR-2 at concentrations used in vivo.

Applicants studied the effects of HBD on leukostasis in a mouse OIR model regarding whether recombinant HBD is able to reduce leukocyte recruitment caused by an increase in hypoxia-induced VEGF164 isoform expression in the eye (Ishida, S., et al.,. J Exp Med, 2003. 198(3): p. 483-9). Leukocyte adhesion at P14 was elevated in wild-type mice but not in VEGF120/188 mice in a model of oxygen-induced retinopathy (OIR). To test the effect of soluble HBD on leukostasis in this model, wildtype mouse pups were taken out of the oxygen chamber on P12 and injected intraperitoneally with 2 nmol of the peptide on day P12 and P13 (hypoxic phase). In addition, two control groups received 5 mg/kg of anti-VEGF neutralizing antibody or 5 mg/kg of a goat IgG control on P12 and P13. Adherent leukocytes inside the retinal vessels of all mice were visualized and counted 48 hours after the first injection at P14.

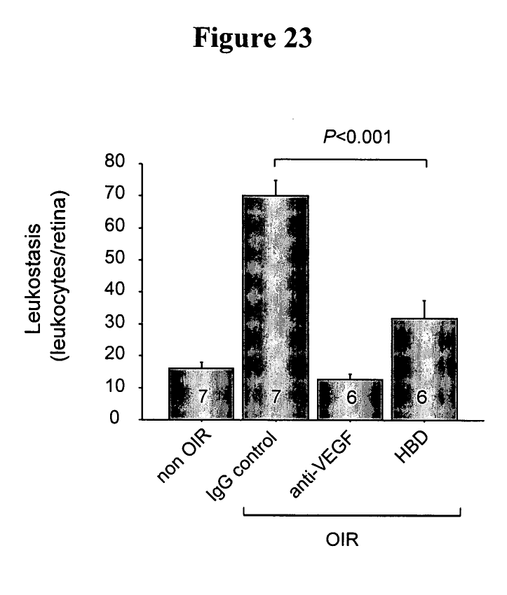

As summarized in FIG. 23, P14 mice in the non OIR control group exhibited low levels of leukostasis in the retina. The number of leukocytes was increased 4.5 fold in OIR mice injected with IgG from non-immunized goats as an isotype control for the goat anti-VEGF neutralizing antibody. These levels are similar to those obtained from non-injected OIR mice demonstrating that the control antibody has no effect on leukocyte behavior. When OIR mice were injected with recombinant HBD and analyzed 48 hours later, a reduction of leukocyte adhesion compared to the OIR control was observed. Pan VEGF isoform blockade was achieved by injecting a neutralizing antibody and resulted in a further inhibition of leukocyte recruitment.

These data suggest that ischemia-induced VEGF expression is responsible for the inflammatory response observed in the eyes of OIR mice. Furthermore, they provide indirect evidence that the VEGF heparin-binding domain contributes significantly to the inflammatory response in this animal model of neovascularization. It would be interesting to see whether suppression of leukostasis by HBD also results in a reduction of pathological neovascularization (preretinal tuft formation), since leukostasis and subsequent pathological vessel growth was not observed in VEGF120/188 mice.

### Example 9

Comparable Binding to Biological Matrices by VEGF164 Variants

Applicants examined the ability of the HBD mutants to bind to biological matrices using cell membrane-integrated proteoheparan sulfates (HSPGs). HSPGs rather than heparin are the natural binding partners for VEGF on cell surfaces and the extracellular matrix in vivo.

Binding of VEGF variants to the cell surface and cross-sections of the eye: Porcine aortic endothelial (PAE) cells were seeded at 3.0×105 cells/well in 12-well dishes and were cultured for 24 h. Cells were washed once with binding buffer (Ham's F-12K medium containing 0.1% (w/v) BSA, pH 7.5, Gibco BRL, CA). Binding of purified mouse VEGF variants (7.14 nM) to the cell surface and matrix was carried out in binding buffer for 30 min at 37° C and 5% CO2. After the binding period, unbound VEGF was removed and the cells were washed three times with binding buffer before bound VEGF was enzymatically dissociated from heparan sulfate proteoglycans on the cell surface and matrix. To this end, heparinase I and III (Sigma, Mo.) were prepared immediately before each experiment by dissolving in 20 mM Tris-HCl (pH 7.5), containing 50 mM NaCl, 4 mM CaCl2, and 0.01% (w/v) BSA. The heparinase mix was then added to the cells at a final concentration of 0.5μ/ml each, and the cells were incubated for 1 hr at 37° C. and 5% CO₂. The medium of each well was collected with a pipette and cells were washed one time with binding buffer. The concentration of VEGF in the medium after heparinase treatment and the final wash was determined by using the mouse VEGF Quantikine® ELISA kit (R&D Systems, MN) according to the instructions of the manufacturer. Each condition was tested with duplicated samples, and the experiment was repeated three times in order to obtain sufficient data for statistical analysis.

Binding of the VEGF variants to mouse eye sections: Mouse eyes from a two month old C57bl/6 female mouse (Charles River Laboratories, MA) were harvested and fixed on a rocker in 4% PFA overnight at 40 C. The eyes were then washed in PBS for three hours and placed in a 10% sucrose solution in PBS for four hours. The eyes were then placed in a 30% sucrose solution overnight at 40° C. The following day the eyes were placed in OCT embedding compound and frozen on dry ice and stored at −800° C. until sectioned. Slides were thawed out and sections were circled with a pap pen and rehydrated in 1×PBS for Smin. The sections were then incubated in 10 μM of one of the following; VEGF164, VEGF120, R14A/R49A or R13A/R14A/R49A overnight at 40° C. The samples were washed once in PBS for 5 min before being incubated in blocking solution (10% goat serum, 1% BSA, 0.05% Triton X-100 in 1×PBS) for 15 min. The samples were then incubated in goat anti-VEGF antibody (1:100, R&D systems, MN) for 1 hr and washed three times in 1×PBS for 5 min each. Samples were then probed with donkey anti-goat Alexa-Fluor 633 secondary (1:500, Molecular Probes, CA) for 45 min and washed three times in 1×PBS for 5 min each. Mounting of sections was performed using Vectashield with DAPI (Vector Laboratories, CA), coverslipped and sealed with nail polish. The sections were then imaged using an epifluorescence microscope (DMRA2, Leica, Wetzlar, Germany) with a digital CCD camera (Hamatsu, Japan). All images were collected using the same exposure time. Binding of each VEGF variant to eye sections was repeated two times with two separate sections each.

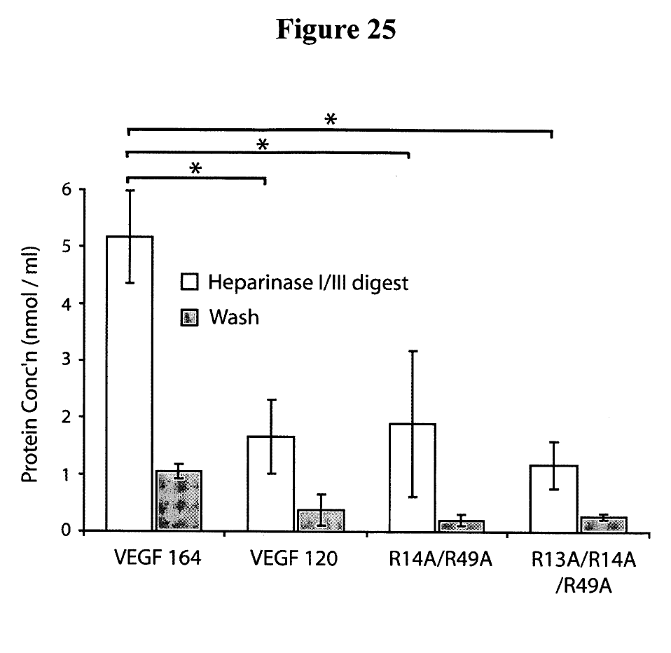

FIG. 25 compares the binding of VEGF120, VEGF164 and HBD mutants to PAE cells. Porcine aortic endothelial cells (3×10⁵ cells) which are devoid of cell-surface VEGF receptors, were incubated with VEGF variants (7.14 nM) and bound VEGF was released from the cell surface and matrix by heparinase digestion (Heparinasel/Ill digest). The amount of VEGF in both digest and wash fraction was determined by a mouse VEGF-specific ELISA. Significantly more VEGF164 bound to the PAE cells than VEGF120 or the heparin-binding deficient mutants R14A/R49A and R13A/R14A/R49A (*P<0.05). Data represent the mean±SD of three independent experiments.

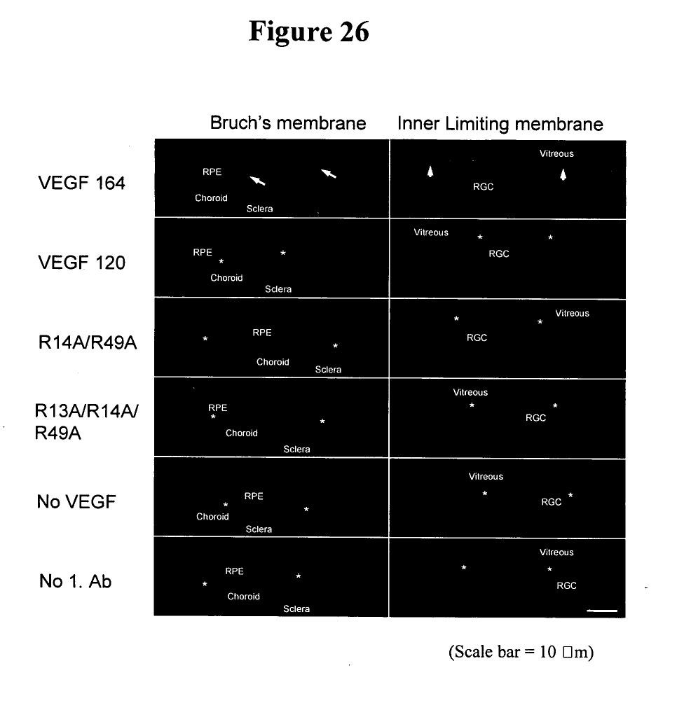

FIG. 26 compares the binding of VEGF variants to biological matrices of the mouse eye. VEGF164 was capable of binding to both Bruch's membrane and the inner limiting membrane (arrows) in the retina. The retinal pigment epithelial layer (RPE), choroid and sclera also exhibited binding by VEGF164 (note that these layers contain some endogenous VEGF as detected in the VEGF control). Only low levels of endogenous VEGF expression was detected in the RPE and RGC cells, however, no labeling of either Bruch's or inner limiting membrane (asterisks) was observed in sections treated with VEGF120. Sections treated with either mutants R14A/R49A or R13A/R14A/R49A showed no binding to either Bruch's or the inner limiting membrane. Staining of eye sections with an anti-VEGF antibody alone to detect endogenous VEGF stain the RPE and RGC layers, while staining of eye sections with secondary antibody alone shows no immunoreactivity. DAPI-staining of nuclei was used in all cases as a marker to determine the appropriate layers of the retina for imaging purposes. The scale bar represents 10 μm.

By using heparinases to release bound VEGF from cell surface of PAE cells, applicants observed significantly more binding of VEGF164 to the cells than VEGF120, confirming the binding of VEGF164 to HSPGs on cell surfaces and matrices (see FIG. 25). R14A/R49A and R13A/R14A/R49A exhibited binding to the cells similar to VEGF120, suggesting that these heparin-binding deficient mutants have lost their binding affinity for heparan sulfate. Binding of HBD mutants to biological matrices was further evaluated in cross sections of the mouse eye. Similar to the cell-binding experiment, VEGF164 but not VEGF120 exhibited prominent binding to the heparan sulfate-rich Bruch's membrane and the inner limiting membrane (ILM) of the eye (see FIG. 26). Both R14A/R49A and R13A/R14A/R49A exhibited no binding to these regions, confirming that mutated arginine residues within the heparin-binding domain of VEGF164 are critical for binding heparan sulfate found in biological matrices.

Incorporation by Reference

The patent and scientific literature referred to herein establishes knowledge that is available to those of skill in the art. All issued patents, patent applications, published foreign applications, and published references, including GenBank database sequences, which are cited herein, are hereby incorporated by reference to the same extent as if each was specifically and individually indicated to be incorporated by reference in their entirety.

Equivalents

Those skilled in the art will recognize, or be able to ascertain, using no more than routine experimentation, numerous equivalents to the specific embodiments described specifically herein. Such equivalents are intended to be encompassed in the scope of the following claims.

We claim:

1. A polypeptide comprising a VEGF polypeptide sequence variant with reduced pro-inflammatory activity having one or more alterations of a native VEGF polypeptide sequence.

2. The polypeptide of claim 1, wherein the alterations of a native VEGF polypeptide sequence reduces heparin binding affinity, while substantially maintaining the affinity for VEGR-2 (FLK-1/KDR).

3. The polypeptide of claim 1, wherein the alterations of a native VEGF polypeptide sequence reduces Neuropilin-1 receptor binding affinity, while substantially maintaining the affinity for VEGR-2 (FLK-1/KDR).

4. The polypeptide of claim 1, wherein the alterations of a native VEGF polypeptide sequence reduces heparin binding affinity, while substantially maintaining the affinity for VEGR-2 (FLK-1/KDR).

5. The polypeptide of claim 1, wherein the alterations of a native VEGF polypeptide sequence reduces Flt-1 binding affinity, while substantially maintaining the affinity for VEGR-2 (FLK-1/KDR).

6. The polypeptide of claim 1, wherein the alterations of a native VEGF polypeptide sequence reduces leukocyte recruitment, while substantially maintaining the function of promoting angiogenesis.

7. The polypeptide of claim 1, wherein the alterations of a native VEGF polypeptide sequence reduces vascular permeability, while substantially maintaining the function of promoting angiogenesis.

8. The polypeptide of claim 1, wherein the alterations of a native VEGF polypeptide sequence reduces leukocyte recruitment, while substantially maintaining the function of promoting neuroprotection.

9. The polypeptide of claim 1, wherein the alterations of a native VEGF polypeptide sequence reduces vascular permeability, while substantially maintaining the function of promoting neuroprotection.

10. The polypeptide of claim 1, wherein the native VEGF polypeptide sequence is a VEGF isoform of a mammal selected from the group consisting of: human, a mouse, a rat, a monkey, a cow, a pig, a sheep, a dog, a cat, and a rabbit.

11. The polypeptide of claim 1, wherein the native VEGF polypeptide sequence is selected form the group consisting of VEGF164, VEGF165, VEGF189, and VEGF206.

12. The polypeptide of claim 1, wherein the native VEGF polypeptide sequence is: PCSERRKHLF VQDPQTCKCS CKNTDSRCKA RQLELNERTC RCDKPRR (Seq. ID No. 1).

13. The polypeptide of claim 12, wherein the VEGF polypeptide sequence variant has the sequence: PCSE X₁X₂X₃ X₄LF VQDPQTCX₅CS CX₆NTDS X₇C X₈A X₉QLELNE X₁₀TC X₁₁CDX₁₂P X₁₃X₁₄ (Seq. ID No.2), wherein at least one of X₁—X₁₄ is a non-basic amino acid substitution, a non-basic amino acid insertion, an amino acid deletion, or a combination thereof, of the native VEGF polypeptide sequence:

| | |
|---|---|
| PCSERRKHLF VQDPQTCKCS CKNTDSRCKA | (Seq. ID No. 1) |
| RQLELNERTC RCDKPRR. | |

14. The polypeptide of claim 13, wherein at least one of X₁—X₁₄ is a non-basic amino acid substitution.

15. The polypeptide of claim 13, wherein at least one of X₁, X₂ and X₅—X₁₁ is a non-basic amino acid substitution.

16. The polypeptide of claim 13, wherein the non-basic amino acid is selected from the group consisting of A, N, D, C, Q, E, I, L, M, S, T, and V.

17. The polypeptide of claim 13, wherein the non-basic amino acid is alanine.

18. The polypeptide of claim 12, wherein the VEGF polypeptide sequence variant has the sequence: PCSEX₁X₂KHLF VQDPQTCKCS CKNTDSRCKA RQLELNERTC X₃CDKPRR (Seq. ID No.28), wherein at least one of X₁, X₂, and X₃ is a non-basic amino acid.

19. The polypeptide of claim 18, wherein the non-basic amino acid is selected from the group consisting of A, N, D, C, Q, E, I, L, M, S, T, and V.

20. The polypeptide of claim 18, wherein the non-basic amino acid is alanine.

21. The polypeptide of claim 18, wherein the VEGF variant has a sequence selected from the group consisting of: PCSERAKHLF VQDPQTCKCS CKNTDSRCKA RQLELNERTC ACDKPRR (Seq. ID No. 3); PCSEAAKHLF VQDPQTCKCS CKNTDSRCKA RQLELNERTC ACDKPRR (Seq. ID No. 4); PCSERRKHLF VQDPQTCKCS CANTDSACKA AQLELNERTC RCDKPRR (Seq. ID No. 5); PCSERRKHLF VQDPQTCKCS CKNTDSACKA AQLELNERTC RCDKPRR (Seq. ID No. 6); PCSERRKHLF VQDPQTCKCS CANTDSRCKA RQLELNERTC RCDKPRR (Seq. ID No. 7); PCSERRKHLF VQDPQTCKCS CANTDSACKA AQLELNERTC ACDKPRR (Seq. ID No. 8); PCSERRKHLF VQDPQTCKCS CANTDSRCKA RQLELNERTC RCDKPRR (Seq. ID No. 9); PCSERRKHLF VQDPQTCKCS CKNTDSRCKA RQLELNEATC ACDKPRR (Seq. ID No. 10); PCSEAAKHLF VQDPQTCKCS CKNTDSRCKA RQLELNERTC RCDKPRR (Seq. ID No. 11); and PCSEAAKHLF VQDPQTCKCS CKNTDSRCKA RQLELNEATC ACDKPRR (Seq. ID No. 12).

22. The polypeptide of claim 18, wherein the VEGF variant has a sequence selected from the group consisting of: ARQENPCGPC SERAKHLFVQ DPQTCKCSCK NTDSRCKARQ LELNERTCAC DKPRR (Seq. ID No. 13); ARQENPCGPC SEAAKHLFVQ DPQTCKCSCK NTDSRCKARQ LELNERTCAC DKPRR (Seq. ID No. 14); ARQENPCGPC SERRKHLFVQ DPQTCKCSCA NTDSACKAAQ LELNERTCRC DKPRR (Seq. ID No. 15); ARQENPCGPC SERRKHLFVQ DPQTCKCSCK NTDSACKAAQ LELNERTCRC DKPRR (Seq. ID No. 16); ARQENPCGPC SERRKHLFVQ DPQTCKCSCA NTDSRCKARQ LELNERTCRC DKPRR (Seq. ID No. 17); ARQENPCGPC SERRKHLFVQ DPQTCKCSCA NTDSACKAAQ LELNERTCAC DKPRR (Seq. ID No. 18); ARQENPCGPC SERRKHLFVQ DPQTCKCSCA NTDSRCKARQ LELNERTCRC DKPRR (Seq. ID No. 19); ARQENPCGPC SERRKHLFVQ DPQTCKCSCK NTDSRCKARQ LELNEATCAC DKPRR (Seq. ID No. 20); ARQENPCGPC SEAAKHLFVQ DPQTCKCSCK NTDSRCKARQ LELNERTCRC DKPRR (Seq. ID No. 21); and ARQENPCGPC SEAAKHLFVQ DPQTCKCSCK NTDSRCKARQ LELNEATCAC DKPRR (Seq. ID No. 22).

23. The polypeptide of claim 18, wherein the VEGF variant has a sequence selected from the group consisting of:

| | |
|---|---|
| APMA EGGGQNHHEV VKFMDVYQRS | (Seq. ID No. 23) |
| YCHPIETLVD IFQEYPDEIE YIFKPSCVPL | |
| MRCGGCCNDE GLECVPTEES NITMQIMRIK | |
| PHQGQHIGEM SFLQHNKCEC RPKKDRARQE | |
| NPCGPC SERAKHLFVQ DPQTCKCSCK | |
| NTDSRCKARQ LELNERTCAC DKPRR; | |
| APMA EGGGQNHHEV VKFMDVYQRS | (Seq. ID No. 24) |
| YCHPIETLVD IFQEYPDEIE YIFKPSCVPL | |
| MRCGGCCNDE GLECVPTEES NITMQIMRIK | |
| PHQGQHIGEM SFLQHNKCEC RPKKDRARQE | |
| NPCGPC SEAAKHLFVQ DPQTCKCSCK | |
| NTDSRCKARQ LELNERTCAC DKPRR; | |
| APMA EGGGQNHHEV VKFMDVYQRS | (Seq. ID No. 25) |
| YCHPIETLVD IFQEYPDEIE YIFKPSCVPL | |
| MRCGGCCNDE GLECVPTEES NITMQIMRIK | |
| PHQGQHIGEM SFLQHNKCEC RPKKDRARQE | |
| NPCGPC SERRKHLFVQ DPQTCKCSCK | |
| NTDSRCKARQ LELNERTCAC DKPRR; | |
| APMA EGGGQNHHEV VKFMDVYQRS | (Seq. ID No. 26) |
| YCHPIETLVD IFQEYPDEIE YIFKPSCVPL | |
| MRCGGCCNDE GLECVPTEES NITMQIMRIK | |
| PHQGQHIGEM SFLQHNKCEC RPKKDRARQE | |
| NPCGPC SERAKHLFVQ DPQTCKCSCK | |
| NTDSRCKARQ LELNERTCRC DKPRR; | |
| and | |
| APMA EGGGQNHHEV VKFMDVYQRS | (Seq. ID No. 27) |
| YCHPIETLVD IFQEYPDEIE YIFKPSCVPL | |
| MRCGGCCNDE GLECVPTEES NITMQIMRIK | |
| PHQGQHIGEM SFLQHNKCEC RPKKDRARQE | |
| NPCGPC SEARKHLFVQ DPQTCKCSCK | |
| NTDSRCKARQ LELNERTCRC DKPRR. | |

24. The polypeptide of claim 13, wherein at least one of X₁—X₁₄ is an amino acid deletion.

25. The polypeptide of claim 13, wherein at least one of X₁, X₂ and X₅—X₁₁ is an amino acid deletion.

26. The polypeptide of claim 25, wherein the VEGF variant has a sequence selected from the group consisting of:

| | |
|---|---|
| APMA EGGGQNHHEV VKFMDVYQRS | (Seq. ID No. 29) |
| YCHPIETLVD IFQEYPDEIE YIFKPSCVPL | |
| MRCGGCCNDE GLECVPTEES NITMQIMRIK | |
| PHQGQHIGEM SFLQHNKCEC RPKKDRARQE | |
| NPCGPC SERKHLFVQ DPQTCKCSCK | |
| NTDSRCKARQ LELNERTCC DKPRR; | |
| APMA EGGGQNHHEV VKFMDVYQRS | (Seq. ID No. 30) |
| YCHPIETLVD IFQEYPDEIE YIFKPSCVPL | |
| MRCGGCCNDE GLECVPTEES NITMQIMRIK | |
| PHQGQHIGEM SFLQHNKCEC RPKKDRARQE | |
| NPCGPC SEKHLFVQ DPQTCKCSCK | |
| NTDSRCKARQ LELNERTCC DKPRR; | |
| APMA EGGGQNHHEV VKFMDVYQRS | (Seq. ID No. 31) |
| YCHPIETLVD IFQEYPDEIE YIFKPSCVPL | |
| MRCGGCCNDE GLECVPTEES NITMQIMRIK | |
| PHQGQHIGEM SFLQHNKCEC RPKKDRARQE | |
| NPCGPC SERRKHLFVQ DPQTCKCSCK | |
| NTDSRCKARQ LELNERTCC DKPRR | |
| and | |
| APMA EGGGQNHHEV VKFMDVYQRS | (Seq. ID No. 32) |
| YCHPIETLVD IFQEYPDEIE YIFKPSCVPL | |
| MRCGGCCNDE GLECVPTEES NITMQIMRIK | |
| PHQGQHIGEM SFLQHNKCEC RPKKDRARQE | |
| NPCGPC SERKHLFVQ DPQTCKCSCK | |
| NTDSRCKARQ LELNERTCRC DKPRR. | |

27. The polypeptide of claim 13, wherein at least one of X₁—X₁₄ is a non-basic amino acid insertion.

28. The polypeptide of claim 13, wherein at least one of X₁, X₂ and X₅—X₁₁ is a non-basic amino acid insertion.

29. The polypeptide of claim 28, wherein the VEGF variant has a sequence selected from the group consisting of:

| | |
|---|---|
| APMA EGGGQNHHEV VKFMDVYQRS | (Seq. ID No. 33) |
| YCHPIETLVD IFQEYPDEIE YIFKPSCVPL | |
| MRCGGCCNDE GLECVPTEES NITMQIMRIK | |
| PHQGQHIGEM SFLQHNKCEC RPKKDRARQE | |
| NPCGPC SERARKHLFVQ DPQTCKCSCK | |
| NTDSRCKARQ LELNERTCARC DKPRR; | |
| APMA EGGGQNHHEV VKFMDVYQRS | (Seq. ID No. 34) |
| YCHPIETLVD IFQEYPDEIE YIFKPSCVPL | |
| MRCGGCCNDE GLECVPTEES NITMQIMRIK | |
| PHQGQHIGEM SFLQHNKCEC RPKKDRARQE | |
| NPCGPC SEARARKHLFVQ DPQTCKCSCK | |
| NTDSRCKARQ LELNERTCARC DKPRR; | |
| APMA EGGGQNHHEV VKFMDVYQRS | (Seq. ID No. 35) |
| YCHPIETLVD IFQEYPDEIE YIFKPSCVPL | |
| MRCGGCCNDE GLECVPTEES NITMQIMRIK | |
| PHQGQHIGEM SFLQHNKCEC RPKKDRARQE | |
| NPCGPC SERRKHLFVQ DPQTCKCSCK | |
| NTDSRCKARQ LELNERTCARC DKPRR; | |
| APMA EGGGQNHHEV VKFMDVYQRS | (Seq. ID No. 36) |
| YCHPIETLVD IFQEYPDEIE YIFKPSCVPL | |
| MRCGGCCNDE GLECVPTEES NITMQIMRIK | |
| PHQGQHIGEM SFLQHNKCEC RPKKDRARQE | |
| NPCGPC SERARKHLFVQ DPQTCKCSCK | |
| NTDSRCKARQ LELNERTCRC DKPRR; | |
| and | |
| APMA EGGGQNHHEV VKFMDVYQRS | (Seq. ID No. 37) |
| YCHPIETLVD IFQEYPDEIE YIFKPSCVPL | |
| MRCGGCCNDE GLECVPTEES NITMQIMRIK | |
| PHQGQHIGEM SFLQHNKCEC RPKKDRARQE | |
| NPCGPC SEARRKHLFVQ DPQTCKCSCK | |
| NTDSRCKARQ LELNERTCRC DKPRR. | |

30. The polypeptide of claims 1, wherein the polypeptide is encoded by a nucleic acid that hybridizes under stringent conditions to a nucleic acid that encodes a native mammalian VEGF cDNA.

31. The polypeptide of claim 30, wherein the native mammalian VEGF cDNA is the human VEGF cDNA of GenBank Accession No. NM_{—}003376.

32. A method of treating a disease or disorder with a VEGF polypeptide sequence variant having reduced inflammatory side effects comprising administering a polypeptide of claim 1.

33. The method of claim 32, wherein the VEGF polypeptide sequence variant increases collateral vessel formation in ischemic heart disease.

34. The method of claim 32, wherein the disease or disorder is wound healing.

35. The method of claim 32, wherein the disease or disorder is a cardiovascular disease.

36. The method of claim 32, wherein the disease or condition is ischemia.

37. The method of claim 32, wherein the VEGF polypeptide sequence variant increases neuroprotection.

38. The method of claim 32, wherein the disease or disorder is a neural disease or disorder.

39. The method of claim 32, wherein the disease or disorder is an ocular neural disease or disorder.

40. The method of claim 32, wherein the disease or disorder is glaucoma.

41. A method of identifying an inhibitor of a heparin/VEGF interaction comprising:
(a) detecting a level of heparin/VEGF interaction in the presence of a test compound; and
(b) comparing the level of heparin/VEGF interaction in the presence of the test compound to the level of heparin/VEGF interaction in the absence of the test compound,
wherein the test compound is an inhibitor of the heparin/VEGF interaction if the level of heparin/VEGF interaction in the presence of a test compound is lower than the level of heparin/VEGF interaction in the absence of the test compound.

42. The method of claim 41, further comprising:
(c) identifying a specific inhibitor of a VEGF pro-inflammatory effect that does not interfere with a VEGF pro-angiogenic effect by detecting a level of VEGF interaction with a VEGF receptor in the presence of the test compound, and
(d) comparing the level of VEGF interaction with the VEGF receptor in the presence of the test compound with the level of VEGF interaction with the VEGF receptor in the absence of the test compound,
wherein the test compound is a specific inhibitor of a VEGF pro-inflammatory effect if the level of VEGF interaction with the VEGF receptor in the presence of the test compound is substantially the same or greater than the level of VEGF interaction with the VEGF receptor in the absence of the test compound, and the test compound is an inhibitor of a heparin/VEGF interaction.

43. The method of claim 42, wherein the VEGF receptor is VEGFR-2 (FLK-1/KDR).

44. The method of claim 42, wherein the VEGF receptor is VEGFR-1.

45. The method of claim 42, wherein the test compound is an aptamer.

46. The method of claim 42, wherein the test compound is a peptide or a peptidomimetic.

47. The method of claim 42, wherein the test compound is a small-molecule.

48. The method of claim 42, further comprising co-administering a VEGF polypeptide and the specific inhibitor of a VEGF pro-inflammatory effect that does not interfere with a VEGF pro-angiogenic effect to a mammalian subject to stimulate angiogenesis with a reduced VEGF pro-inflammatory effect.

49. A method of isolating a VEGF polypeptide sequence variant having a reduced affinity for heparin comprising:
(a) providing a polypeptide comprising a variant of a native VEGF polypeptide sequence; and
(b) comparing the level of heparin binding of the polypeptide comprising the variant to the level of heparin binding of the polypeptide comprising the native VEGF polypeptide sequence,
wherein the VEGF polypeptide sequence variant is a VEGF polypeptide sequence variant having a reduced affinity for heparin if the level of heparin binding of the polypeptide comprising the variant is lower than the level of heparin binding of the polypeptide comprising the native VEGF polypeptide sequence.

50. A method for identifying a potential modulator of VEGF heparin binding domain activity, comprising the steps of:
(a) providing the atomic co-ordinates of the site responsible for VEGF heparin binding domain function, thereby defining a three-dimensional structure of the site responsible for VEGF heparin binding;
(b) using the three dimensional structure of the VEGF heparin binding domain to design or select a potential modulator by computer modeling;
(c) providing the potential modulator; and
(d) physically contacting the potential modulator with the VEGF heparin binding domain to determine the ability of said potential modulator to modulate VEGF heparin binding domain activity,
wherein a modulator of the VEGF heparin binding domain activity is identified.

51. An isolated nucleic acid molecule comprising a sequence that encodes a VEGF variant comprising the polypeptide of claim 1.

52. An expression vector for producing a VEGF variant comprising the polypeptide of claim 1, in a host cell, said vector comprises:
a) a polynucleotide encoding the VEGF variant;
b) transcriptional and translational regulatory sequences functional in said host cell operably linked to said VEGF variant-encoding polynucleotide; and
c) a selectable marker.

53. A host cell stably transformed and transfected with a polynucleotide encoding a VEGF variant comprising the polypeptide of claim 1, in a manner allowing the expression in said host cell of the VEGF variant.

54. A method of inhibiting VEGF164 induced leukostasis comprising the step of administering a soluble heparin binding domain.

55. The method of claim 54, wherein, the soluble heparin binding domain comprises a polypeptide having the sequence of ARQENPCGPC SERRKHLFVQ DPQTCKCSCK NTDSRCKARQ LELNERTCRC DKPRR (Seq. ID No.38).
